(19) United States
(12) Patent Application Publication
(10) Pub. No.: US 2025/0313905 A1
(43) Pub. Date: Oct. 9, 2025

(54) METHODS AND COMPOSITIONS FOR IMPROVED PSYCHOLOGICAL AND RESILIENCE MEASURES

(71) Applicant: The Regents of The University of California, Oakland, CA (US)

(72) Inventors: Arpana Gupta, Los Angeles, CA (US); Tien Dong, Los Angeles, CA (US)

(21) Appl. No.: 18/866,836

(22) PCT Filed: May 19, 2023
(86) PCT No.: PCT/US2023/067223

§ 371 Date: Nov. 18, 2024

(51) Int. Cl. C12Q 1/689; C12Q 1/6874

(52) U.S. Cl. CPC C12Q 1/689; C12Q 1/6874; C12Q 2600/118

(57) ABSTRACT

Provided herein are methods of improving mood and/or ameliorating a depressive symptom, treating or preventing a psychiatric or neurologic disorder, and/or enhancing the beneficial effects of physical activity in a subject by administering to the subject i) a composition comprising bacteria of at least one genus selected from Megasphaera, Methanobrevibacter, Christensenellaceae, Prevotella, Fournierella, Acidaminococcus or Akkermansia, ii) a metabolite produced by a strain of Megasphaera, Methanobrevibacter, Christensenellaceae, Prevotella, Fournierella, Acidaminococcus or Akkermansia bacteria, iii) a composition comprising myoinositol, or iv) an agent that inhibits the activity of or lowers the levels of cytidine in the GI tract of the subject.

### RELATED APPLICATIONS

This application claims the benefit of priority to U.S. Provisional Patent Application Ser. No. 63/343,800, filed May 19, 2022, which is hereby incorporated by reference in its entirety.

### GOVERNMENT SUPPORT

This invention was made with government support under DK121025, DK007180, DK106528, and MD015904 awarded by the National Institutes of Health. The government has certain rights in the invention.

### BACKGROUND

In the past few decades, obesity rates have rapidly grown to epidemic proportions both in developed countries and worldwide, with an estimated 650 million adults considered obese. In the United States alone, 42.4% of adults were found to be obese in 2020 and these trends are expected to continue rising. Obesity leads not only to the development of physical comorbidities such as cardiovascular disease, diabetes mellitus, and various cancers, but also to mental health consequences due to stress from weight discrimination and stigmatization^{3,4}. Individuals who have reported weight-related discrimination are more likely to engage in high-risk behaviors such as drug abuse and cigarette smoking, and also tend to experience greater weight gain over time. Additionally, there is a greater likelihood of developing maladaptive eating behaviors, including food addiction, binge-eating, emotional eating, and increased consumption of calories.

### SUMMARY

Provided herein are methods of improving mood and/or ameliorating a depressive symptom in a subject. In some embodiments, the methods include administering a composition comprising bacteria of at least one genus selected from Megasphaera, Methanobrevibacter, Christensenellaceae, Prevotella, Fournierella, Acidaminococcus, and Akkermansia to the subject. The method may include administering at least two, at least three, at least four, at least five, at least six, or all seven bacterial genera to the subject.

Also provided herein are methods of preventing or treating a psychiatric or neurologic disorder in a subject. In some embodiments, the methods include administering a composition comprising bacteria of at least one genus selected from Megasphaera, Methanobrevibacter, Christensenellaceae, Prevotella, Fournierella, Acidaminococcus and Akkermansia to the subject. The method may include administering at least two, at least three, at least four, at least five, at least six, or all seven bacterial genera to the subject.

In some embodiments, the administration of the composition raises the levels of at least one genus of bacteria selected from Megasphaera, Methanobrevibacter, Christensenellaceae, Prevotella, Fournierella, Acidaminococcus and Akkermansia in the gastrointestinal (GI) tract of the subject. The administration may raise the level of at least two, at least three, at least four, at least five, at least six, or at least seven bacteria genera in the GI tract of the subject.

In some embodiments, the method further comprises administering an antibiotic to the subject prior to administration of Megasphaera, Methanobrevibacter, Christensenellaceae, Prevotella, Fournierella, Acidaminococcus and/or Akkermansia bacteria.

In some embodiments, the composition comprises bacteria of at least one genus (e.g., at least two, at least three, at least four, at least five, at least six, or all seven genera) selected from Megasphaera, Methanobrevibacter, Christensenellaceae, Prevotella, Fournierella, Acidaminococcus and Akkermansia. In other embodiments, the methods comprise administering at least one genus (e.g., at least two, at least three, at least four, at least five, at least six, or all seven genera) selected from Megasphaera, Methanobrevibacter, Christensenellaceae, Prevotella, Fournierella, Acidaminococcus and Akkermansia in multiple compositions.

Provided herein are methods of improving mood and/or ameliorating a depressive symptom in a subject. In some embodiments, the methods include administering a composition comprising bacteria of at least one genus selected from Megasphaera, Methanobrevibacter, Christensenellaceae, Prevotella and Akkermansia to the subject. The method may include administering at least two, at least three, at least four, or all five bacterial genera to the subject.

Also provided herein are methods of preventing or treating a psychiatric or neurologic disorder in a subject. In some embodiments, the methods include administering a composition comprising bacteria of at least one genus selected from Megasphaera, Methanobrevibacter, Christensenellaceae, Prevotella and Akkermansia to the subject. The method may include administering at least two, at least three, at least four, or all five bacterial genera to the subject.

In some embodiments, the administration of the composition raises the levels of at least one genus of bacteria selected from Megasphaera, Methanobrevibacter, Christensenellaceae, Prevotella and Akkermansia in the gastrointestinal (GI) tract of the subject. The administration may raise the level of at least two, at least three, at least four, or all five bacteria genera in the GI tract of the subject.

In some embodiments, the method further comprises administering an antibiotic to the subject prior to administration of Megasphaera, Methanobrevibacter, Christensenellaceae, Prevotella or Akkermansia bacteria.

In some embodiments, the composition comprises bacteria of at least one genus (e.g., at least two, at least three, at least four, or all five genera) selected from Megasphaera, Methanobrevibacter, Christensenellaceae, Prevotella and Akkermansia. In other embodiments, the methods comprise administering at least one genus (e.g., at least two, at least three, at least four, or all five genera) selected from Megasphaera, Methanobrevibacter, Christensenellaceae, Prevotella and Akkermansia in multiple compositions.

The Methanobrevibacter bacteria may be Methanobrevibacter smithii.

Provided herein are methods of improving mood and/or ameliorating a depressive symptom in a subject, the method comprising administering a composition comprising myoinositol to the subject. The method may comprise administering an antibiotic to the subject prior to administration of myoinositol to the subject.

In some aspects, provided herein are methods of improving mood and/or ameliorating a depressive symptom in a subject, the method comprising administering to the subject an agent that inhibits the activity of or lowers the levels of cytidine in the GI tract of the subject. The agent may be a small molecule or a peptide. In some embodiments, the method further comprises administering an antibiotic to the subject prior to administration of the agent to the subject.

Also provided herein are methods of improving mood and/or ameliorating a depressive symptom in a subject, the method comprising administering a composition comprising at least one of the metabolites listed in Table 12 or Table 13 to the subject. In some embodiments, the method further comprises administering an antibiotic to the subject prior to administration of at least one of the metabolites listed in Table 12 or Table 13 to the subject. Also provided herein are methods of improving mood and/or ameliorating a depressive symptom in a subject, the method comprising administering an agent to the subject that inhibits the activity of or lowers the levels of Ruminococcus gnavus in the GI tract of the subject (e.g., administering an agent, that is an antibiotic that targets Ruminococcus gnavus, such as an antibiotic that specifically targets Ruminococcus gnavus). The agent may be a small molecule, a peptide, and/or an antibiotic.

The depressive symptom may be selected from anxiety, apathy, general discontent, guilt, hopelessness, loss of interest or pleasure in activities, mood swings, sadness, agitation, excessive crying, irritability, restlessness, social isolation, early awakening, excess sleepiness, insomnia, restless sleep, excessive hunger, fatigue, loss of appetite, lack of concentration, slowness in activity, suicidal ideation, weight gain and weight loss.

In some embodiments, the subject is afflicted with or at risk for a psychiatric or neurologic disorder, such as bipolar depression, post-partum depression (PPD), post-traumatic stress disorder (PTSD), major depressive disorder (MDD), or food addiction.

Also provided herein are methods of treating or preventing a psychiatric or neurologic disorder in a subject, the method comprising administering to the subject an agent that inhibits the activity of or lowers the levels of cytidine in the GI tract of the subject.

In some aspects, provided herein are methods of treating or preventing a psychiatric or neurologic disorder in a subject, the method comprising administering a composition comprising myoinositol to the subject.

Also provided herein are methods of treating or preventing a psychiatric or neurologic disorder in a subject, the method comprising administering a composition comprising at least one of the metabolites listed in Table 12 or Table 13 to the subject.

In some aspects, provided herein are methods of treating or preventing a psychiatric or neurologic disorder in a subject, the method comprising administering an agent to the subject that inhibits the activity of or lowers the levels of Ruminococcus gnavus in the GI tract of the subject.

In some embodiments, the composition is formulated for oral delivery.

The methods described herein may include administering an additional therapy for a psychiatric or neurologic disorder.

In some aspects, provided herein are methods for determining whether a subject is at risk for a psychiatric or neurologic disorder, the method comprising: obtaining a sample from the GI tract of the subject, optionally isolating microbial DNA from the sample, identifying the amount of at least one genus of bacteria selected from Megasphaera, Methanobrevibacter, Christensenellaceae, Prevotella and Akkermansia in the microbiome of the sample, and if the microbiome comprises a lower level of Megasphaera, Methanobrevibacter, Christensenellaceae, Prevotella or Akkermansia bacteria compared to a control level, the subject is considered at risk for the psychiatric or neurologic disorder.

In some embodiments, the control level is a level measured in a sample from the GI tract of the subject taken earlier in time. In other embodiments, the control level is a mean or median level of Megasphaera, Methanobrevibacter, Christensenellaceae, Prevotella or Akkermansia bacteria in subjects not afflicted with or at risk for the psychiatric or neurologic disorder.

Also provided herein are methods of treating or preventing a psychiatric or neurologic disorder in a subject and/or improving mood and/or ameliorating a depressive symptom in a subject by administering a composition comprising a metabolite produced by a strain of Megasphaera, Methanobrevibacter, Christensenellaceae, Prevotella or Akkermansia bacteria to the subject, e.g., a metabolite selected from bacterial culture supernatant, cell lysate, proteins, nucleic acids, lipids, and other bacterial molecules.

The psychiatric or neurologic disorder may be depression, PPD, PTSD, MDD, food addiction, or bipolar disorder.

In some aspects, provided herein are methods of enhancing the beneficial effects of physical activity in a subject, the method comprising administering a composition comprising bacteria of at least one genus selected from Megasphaera, Methanobrevibacter, Christensenellaceae, Prevotella or Akkermansia to the subject, administering to the subject an agent that inhibits the activity of or lowers the levels of cytidine in the GI tract of the subject, and/or administering a composition comprising myoinositol to the subject.

In some aspects, provided herein are methods of enhancing the beneficial effects of physical activity in a subject, the method comprising administering a composition comprising a metabolite produced by a strain of Megasphaera, Methanobrevibacter, Christensenellaceae, Prevotella or Akkermansia bacteria to the subject, e.g., a metabolite selected from bacterial culture supernatant, cell lysate, proteins, nucleic acids, lipids, and other bacterial molecules.

In some aspects, provided herein are methods for determining whether a subject is at risk for a psychiatric or neurologic disorder, the method comprising: obtaining a sample from the GI tract of the subject, optionally isolating microbial DNA from the sample, identifying the amount of at least one genus of bacteria selected from Megasphaera, Methanobrevibacter, Christensenellaceae, Prevotella, Fournierella, Acidaminococcus and Akkermansia in the microbiome of the sample, and if the microbiome comprises a lower level of Megasphaera, Methanobrevibacter, Christensenellaceae, Prevotella, Fournierella, Acidaminococcus or Akkermansia bacteria compared to a control level, the subject is considered at risk for the psychiatric or neurologic disorder.

In some embodiments, the control level is a level measured in a sample from the GI tract of the subject taken earlier in time. In other embodiments, the control level is a mean or median level of Megasphaera, Methanobrevibacter, Christensenellaceae, Prevotella, Fournierella, Acidaminococcus or Akkermansia bacteria in subjects not afflicted with or at risk for the psychiatric or neurologic disorder.

Also provided herein are methods of treating or preventing a psychiatric or neurologic disorder in a subject and/or improving mood and/or ameliorating a depressive symptom in a subject by administering a composition comprising a metabolite produced by a strain of Megasphaera, Methanobrevibacter, Christensenellaceae, Prevotella, Fournierella, Acidaminococcus or Akkermansia bacteria to the subject, e.g., a metabolite selected from bacterial culture supernatant, cell lysate, proteins, nucleic acids, lipids, and other bacterial molecules.

The psychiatric or neurologic disorder may be depression, PPD, PTSD, MDD, food addiction, or bipolar disorder.

In some aspects, provided herein are methods of enhancing the beneficial effects of physical activity in a subject, the method comprising administering a composition comprising bacteria of at least one genus selected from Megasphaera, Methanobrevibacter, Christensenellaceae, Prevotella, Fournierella, Acidaminococcus or Akkermansia to the subject, administering to the subject an agent that inhibits the activity of or lowers the levels of cytidine in the GI tract of the subject, and/or administering a composition comprising myoinositol to the subject.

In some aspects, provided herein are methods of enhancing the beneficial effects of physical activity in a subject, the method comprising administering a composition comprising a metabolite produced by a strain of Megasphaera, Methanobrevibacter, Christensenellaceae, Prevotella, Fournierella, Acidaminococcus or Akkermansia bacteria to the subject, e.g., a metabolite selected from bacterial culture supernatant, cell lysate, proteins, nucleic acids, lipids, and other bacterial molecules.

In some aspects, provided herein are methods of enhancing the beneficial effects of physical activity in a subject, the method comprising a composition comprising at least one of the metabolites listed in Table 12 or Table 13 to the subject.

Also provided herein are methods of enhancing the beneficial effects of physical activity in a subject, the method comprising administering an agent to the subject that inhibits the activity of or lowers the levels of Ruminococcus gnavus in the GI tract of the subject.

Beneficial effects of physical activity include increased Brief Resilience Scale (BRS) score, decreased Yale Food Addiction Scale (YFAS), improved mood, increased acceptance, or increased functional brain connectivity.

### BRIEF DESCRIPTION OF THE FIGURES

FIG. 1 shows study overview, workflow, and results. This figure denotes the overview of workflow and analyses, and summarizes the findings from the study

FIG. 2A-FIG. 2C shows brain connectivity differences based on level of physical activity. FIG. 2D also show a connectogram and the associated brain regions demonstrating q-value significant (<0.05) brain connections derived from FDR correction between high vs. moderate physical activity individuals. FIG. 2F shows a connectogram and associated brain regions demonstrating q-value significant (<0.05) brain connections derived from FDR correction between moderate vs. low physical activity individuals. Green lines denote increased connectivity in the moderate group versus blue lines represent increased connectivity in the low group. Red lines denote increased connectivity in the high group versus green lines represent increased connectivity in the moderate group. FIG. 2E shows the q-value significant brain regions when comparing high versus moderate physical activity are displayed. FIG. 2G shows the q-value significant brain regions when comparing moderate versus low physical activity are displayed.

FIG. 3A-3B shows microbial taxa associated with physical activity. Taxonomic profiles of taxa ≥1% relative abundance between low, moderate, and high physical activity groups. FIG. 3A shows DESEq2 analysis comparing high vs. low physical activity showing 6 OTUs elevated in high physical activity participants and 1 OTU elevated in low physical activity participants. FIG. 3B shows DESEq2 analysis showing several OTUs increased with moderate when compared to low physical activity individuals.

FIG. 4A-FIG. 4C show fecal metabolites associated with physical activity level. FIG. 4A shows boxplots depicting the levels of q-significant fecal metabolites between high versus low physical activity groups on components 1 and 2. FIG. 4B shows boxplots depicting the levels of q-significant fecal metabolites between high versus moderate physical activity groups on components 1 and 2. FIG. 4C shows boxplots depicting the levels of q-value significant fecal metabolites between moderate versus low physical activity groups.

FIG. 5 shows brain connectivity differences based on level of physical activity (PA). FIG. 5A shows clustering plot by SPLS-DA discriminating brain functional connectivity by PA groups. FIG. 5B shows a connectogram demonstrating q-value significant (<0.05) brain connections derived from FDR correction between high vs. moderate PA individuals. Red lines denote increased connectivity in the high group versus green lines represent increased connectivity in the moderate group. FIG. 5C shows a connectogram demonstrating q-value significant (<0.05) brain connections derived from FDR correction between moderate vs. low PA individuals. Green lines denote increased connectivity in the moderate group versus blue lines represent increased connectivity in the low group. FIG. 5D shows the q-value significant brain regions when comparing high versus moderate PA are displayed. FIG. 5E shows the q-value significant brain regions when comparing moderate versus low PA are displayed.

FIG. 6 shows microbial taxa associated with PA. FIG. 6A shows principal coordinate analysis plot of the microbiome showing beta-diversity by PA level encircled by 95% confidence interval ellipses, adjusting for sex, age, BMI, and diet. FIGS. 6B and 6C shows a box plot of microbial alpha-diversity by Shannon index and Chao index respectively across PA groups. FIG. 6D shows MaAslin2 analysis comparing high vs. low PA showing three genera elevated in high PA participants and two genera elevated in low PA participants. FIG. 6E shows MaAslin2 analysis showing one genus increased and seven decreased with moderate when compared to low PA individuals. FIG. 6F shows a boxplot depicting the differences in Prevotella to Bacteroides ratio across high, moderate, and low PA groups.

FIG. 7 shows differences in bacterial transcript based on level of PA. FIG. 7A shows clustering plot by SPLS-DA discriminating bacterial transcript by PA groups. FIG. 7B shows differentially abundant bacterial transcripts, annotated by KEGG KO number and gene name, between high versus moderate PA groups. FIG. 7C shows differentially abundant bacterial transcripts, annotated by KEGG KO number and gene name, between high versus low PA groups. FIG. 7D shows differentially abundant bacterial transcripts, annotated by KEGG KO number and gene name, between moderate versus low PA groups.

FIG. 8 shows metabolites associated with PA. Boxplots depicting the fecal metabolites significantly associated with PA across high, moderate, and low PA groups.

### DETAILED DESCRIPTION

The compositions and methods provided herein are based, in part, on the discovery that physical activity can modulate specific gut bacteria and metabolites and that gut bacteria and such metabolites associated with high or moderate physical activity can also be used to treat a psychiatric disorder (e.g., a disorder disclosed herein).

Therefore, provided herein are methods of improving mood and/or ameliorating a depressive symptom, treating or preventing a psychiatric or neurologic disorder, and/or enhancing the beneficial effects of physical activity in a subject, in a subject, the method comprising administering a composition comprising at least one of the metabolites listed in Table 12 or Table 13 to the subject. In some embodiments, the method further comprises administering an antibiotic to the subject prior to administration of at least one of the metabolites listed in Table 12 or Table 13 to the subject. Also provided herein are methods of improving mood and/or ameliorating a depressive symptom in a subject, treating or preventing a psychiatric or neurologic disorder, and/or enhancing the beneficial effects of physical activity in a subject the method comprising administering an agent to the subject that inhibits the activity of or lowers the levels of Ruminococcus gnavus in the GI tract of the subject.

Provided herein are methods of improving mood and/or ameliorating a depressive symptom, treating or preventing a psychiatric or neurologic disorder, and/or enhancing the beneficial effects of physical activity in a subject by administering to the subject i) a composition comprising bacteria of at least one genus selected from Megasphaera, Methanobrevibacter, Christensenellaceae, Prevotella, Fournierella, Acidaminococcus or Akkermansia, ii) a metabolite produced by a strain of Megasphaera, Methanobrevibacter, Christensenellaceae, Prevotella, Fournierella, Acidaminococcus or Akkermansia bacteria, iii) a composition comprising myoinositol, or iv) an agent that inhibits the activity of or lowers the levels of cytidine in the GI tract of the subject.

Provided herein are methods of improving mood and/or ameliorating a depressive symptom in a subject. In some embodiments, the methods include administering a composition comprising bacteria of at least one genus selected from Megasphaera, Methanobrevibacter (e.g., Methanobrevibacter smithii), Christensenellaceae, Prevotella, Fournierella, Acidaminococcus and Akkermansia to the subject.

Also provided herein are methods of preventing or treating a psychiatric or neurologic disorder in a subject. In some embodiments, the methods include administering a composition comprising bacteria of at least one genus selected from Megasphaera, Methanobrevibacter (e.g., Methanobrevibacter smithii), Christensenellaceae, Prevotella, Fournierella, Acidaminococcus and Akkermansia to the subject.

Provided herein are methods of improving mood and/or ameliorating depressive symptoms in a subject, the method comprising administering a composition comprising myoinositol to the subject. The method may comprise administering an antibiotic to the subject prior to administration of myoinositol to the subject.

In some aspects, provided herein are methods of improving mood and/or ameliorating a depressive symptom in a subject, the method comprising administering to the subject an agent that inhibits the activity of or lowers the levels of cytidine in the GI tract of the subject. The agent may be a small molecule or a peptide.

In some embodiments, the methods further comprise administering an antibiotic to the subject prior to administration of the agent to the subject. For example, administration of an antibiotic or microbial agents that does not target a Megasphaera, Methanobrevibacter, Christensenellaceae, Prevotella, Fournierella, Acidaminococcus and Akkermansia bacteria.

Also provided herein are methods of treating or preventing a psychiatric or neurologic disorder in a subject, the method comprising administering to the subject an agent that inhibits the activity of or lowers the levels of cytidine in the GI tract of the subject.

In some aspects, provided herein are methods of treating or preventing a psychiatric or neurologic disorder in a subject, the method comprising administering a composition comprising myoinositol to the subject.

In some aspects, provided herein are methods for determining whether a subject is at risk for a psychiatric or neurologic disorder, the method comprising: obtaining a sample from the GI tract of the subject, optionally isolating microbial DNA from the sample, identifying the amount of at least one genus of bacteria selected from Megasphaera, Methanobrevibacter, Christensenellaceae, Prevotella, Fournierella, Acidaminococcus and Akkermansia in the microbiome of the sample, and if the microbiome comprises a lower level of Megasphaera, Methanobrevibacter, Christensenellaceae, Prevotella, Fournierella, Acidaminococcus or Akkermansia bacteria compared to a control level, the subject is considered at risk for the psychiatric or neurologic disorder.

Also provided herein are methods of treating or preventing a psychiatric or neurologic disorder in a subject by administering a composition comprising a metabolite produced by a strain of Megasphaera, Methanobrevibacter, Christensenellaceae, Prevotella, Fournierella, Acidaminococcus or Akkermansia bacteria to the subject, e.g., a metabolite selected from bacterial culture supernatant, cell lysate, proteins, nucleic acids, lipids, and other bacterial molecules.

In some aspects, provided herein are methods of enhancing the beneficial effects of physical activity in a subject, the method comprising administering a composition comprising bacteria of at least one genus selected from Megasphaera, Methanobrevibacter, Christensenellaceae, Prevotella, Fournierella, Acidaminococcus or Akkermansia to the subject, administering to the subject an agent that inhibits the activity of or lowers the levels of cytidine in the GI tract of the subject, and/or administering a composition comprising myoinositol to the subject.

In some aspects, provided herein are methods of enhancing the beneficial effects of physical activity in a subject, the method comprising administering a composition comprising a metabolite produced by a strain of Megasphaera, Methanobrevibacter, Christensenellaceae, Prevotella, Fournierella, Acidaminococcus or Akkermansia bacteria to the subject, e.g., a metabolite selected from bacterial culture supernatant, cell lysate, proteins, nucleic acids, lipids, and other bacterial molecules.

### Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

The term “agent” is used herein to denote a chemical compound, a small molecule, a mixture of chemical compounds and/or a biological macromolecule (such as a nucleic acid, an antibody, an antibody fragment, a protein or a peptide). Agents may be identified as having a particular activity by screening assays. The activity of such agents may render them suitable as a “therapeutic agent” which is a biologically, physiologically, or pharmacologically active substance (or substances) that acts locally or systemically in a subject.

The term “amino acid” is intended to embrace all molecules, whether natural or synthetic, which include both an amino functionality and an acid functionality and capable of being included in a polymer of naturally occurring amino acids. Exemplary amino acids include naturally occurring amino acids; analogs, derivatives and congeners thereof, amino acid analogs having variant side chains; and all stereoisomers of any of the foregoing.

As used herein, “improving mood” includes, but is not limited to, improving a depressive symptoms disclosed herein, increasing coping strategies such as “acceptance” or “resilience”, and/or increasing or improving scores on clinical tests or diagnostic criteria meant to evaluate an individual's mood or mental state. Resilience is defined as the ability to positively adapt in response to significant adversity or stressors. An example of a clinical test to evaluate mental state/health includes Brief Resilience Scale (BRS) resilience scores. The Brief Resilience Scale assesses the perceived ability to bounce back or recover from stress. The scale was developed to assess a unitary construct of resilience, including both positively and negatively worded items. The possible score range on the BRS is from 1 (low resilience) to 5 (high resilience).

As used herein, “improving a depressive symptom” includes improvement that is self-reported and that which is reported as improved by a clinician (e.g., though a clinical scoring protocol or test.

As used herein, a “psychiatric or neurologic disorder” includes bipolar depression, post-partum depression (PPD), post-traumatic stress disorder (PTSD), major depressive disorder (MDD), or food addiction. Food addiction may be diagnosed by a clinician and/or diagnosed based on the Yale Food Addiction Scale (YFAS). The YFAS allows for a systematic examination of a food addiction. The YFAS includes 25 items and translates the diagnostic criteria for substance dependence as stated in the DSM-IV (American Psychiatric Association, 2000) to relate to the consumption of calorie-dense foods (e.g., high in refined carbohydrates and fat). The scale includes items that assess specific criteria, such as diminished control over consumption, a persistent desire or repeated unsuccessful attempts to quit, withdrawal, and clinically significant impairment. The YFAS includes two scoring options: 1) a “symptom count” ranging from 0 to 7 that reflects the number of addiction-like criteria endorsed and 2) a dichotomous “diagnosis” that indicates whether a threshold of three or more “symptoms” plus clinically significant impairment or distress has been met. For example, a subject may be deemed to be afflicted with a food addiction if they achieve a YFAS symptom count ≥3. Lower numbers may indicate at risk individuals. The term “psychiatric disorder” can also refer to any disease of the mind and includes diseases and disorders listed in the Diagnostic and Statistical Manual of Mental Disorders-Fourth Edition (DSM-IV), published by the American Psychiatric Association, Washington D. C. (1994). Psychiatric disorders include anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder.

As used herein the phrase “enhancing the beneficial effects of physical activity”, include, but are not limited to, increasing the amount of Megasphaera, Methanobrevibacter, Christensenellaceae, Prevotella, Fournierella, Acidaminococcus and Akkermansia or a metabolite produced by Megasphaera, Methanobrevibacter, Christensenellaceae, Prevotella, Fournierella, Acidaminococcus and Akkermansia in the GI tract of the subject; lowering levels of cytidine in the subject; modulating the level of a metabolite listed in Table 11 (e.g., increasing the level of a metabolite in Table 12 or Table 13); increasing the BRS resilience score; lowering YFAS score; improving or ameliorating a symptom of a psychiatric or neurologic disorder described herein, improving an individual's score on any one of the following test: International Physical Activity Questionnaire (IPAQ), Perceived Stress Scale (PSS), Hospital Anxiety and Depression Scale (HADS), Yale Food Addiction Scale (YFAS), Positive and Negative Affect Schedule (PANAS), Brief-COPE (BCope), State-Trait Anxiety Inventory (STAI); increased ability to engage in coping mechanisms such as acceptance; and/or increased functional brain connectivity (see Table 2 for metrics measuring functional brain connectivity).

The term “preventing” is art-recognized, and when used in relation to a condition, such as a local recurrence, is well understood in the art, and includes administration of a composition which reduces the frequency of, or delays the onset of, symptoms of a medical condition in a subject relative to a subject which does not receive the composition. Thus, prevention of acne includes, for example, reducing the number of detectable acne lesions in a population of patients receiving a prophylactic treatment relative to an untreated control population, and/or delaying the appearance of detectable lesions in a treated population versus an untreated control population, e.g., by a statistically and/or clinically significant amount.

The term “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof).

The term “subject” refers to a mammal, including, but not limited to, a human or non-human mammal, such as a bovine, equine, canine, ovine, or feline. In some embodiments, the subject is human.

A “therapeutically effective amount” of a compound with respect to the subject method of treatment refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or prevented, e.g., at a reasonable benefit/risk ratio applicable to any medical treatment.

As used herein, the term “treating” or “treatment” includes reversing, reducing, or arresting the symptoms, clinical signs, and underlying pathology of a condition in a manner to improve or stabilize a subject's condition.

### Therapeutic Methods

Provided herein are methods of improving mood and/or ameliorating a depressive symptom, treating or preventing a psychiatric or neurologic disorder, and/or enhancing the beneficial effects of physical activity in a subject. In some aspects, the methods relate to administering a composition comprising bacteria of at least one genus selected from Megasphaera, Methanobrevibacter, Christensenellaceae, Prevotella, Fournierella, Acidaminococcus or Akkermansia, and/or a metabolite produced by a strain of Megasphaera, Methanobrevibacter, Christensenellaceae, Prevotella, Fournierella, Acidaminococcus or Akkermansia bacteria to the subject, e.g., a metabolite selected from bacterial culture supernatant, cell lysate, proteins, nucleic acids, lipids, and other bacterial molecules to the subject.

Compositions described herein may have at least one species or strain(s) of Megasphaera, Methanobrevibacter, Christensenellaceae, Prevotella, Fournierella, Acidaminococcus and/or Akkermansia. Compositions may contain two or more, three or more, four or more, five or more, six or more, seven or more, eight or more, nine or more, or ten or more species or strains of Megasphaera, Methanobrevibacter, Christensenellaceae, Prevotella, Fournierella, Acidaminococcus and/or Akkermansia.

The methods described herein also include administering at least one (or more) species or strain(s) of Megasphaera, Methanobrevibacter, Christensenellaceae, Prevotella, Fournierella, Acidaminococcus and/or Akkermansia in two or more compositions. In some embodiments, the methods described herein also include administering at least one (or more) species or strain(s) of may contain two or more, three or more, four or more, five or more, six or more, seven or more, eight or more, nine or more, or ten or more species or strains of Megasphaera Methanobrevibacter, Christensenellaceae, Prevotella, Fournierella, Acidaminococcus and/or Akkermansia in two or more compositions.

Provided herein are methods of improving mood and/or ameliorating a depressive symptom, treating or preventing a psychiatric or neurologic disorder, and/or enhancing the beneficial effects of physical activity in a subject, the method comprising administering a composition comprising myoinositol to the subject.

Provided herein are methods of improving mood and/or ameliorating a depressive symptom, treating or preventing a psychiatric or neurologic disorder, and/or enhancing the beneficial effects of physical activity in a subject, the method comprising administering a composition comprising at least one (e.g., at least two, at least three, at least four, at least five, at least six, at least seven, at least eight, at least nine, at least ten, etc.) of the metabolites listed in Table 12 or Table 13 to the subject. In some embodiments, the metabolites are associated with high or moderate physical activity.

Provided herein are methods of improving mood and/or ameliorating a depressive symptom, treating or preventing a psychiatric or neurologic disorder, and/or enhancing the beneficial effects of physical activity in a subject by modulating at least one (e.g., at least two, at least three, at least four, at least five, at least six, at least seven, at least eight, at least nine, at least ten, etc.) of the metabolites listed Table 11 to the subject. Modulating, as used herein, includes, but is not limited to, administering to the subject a metabolite includes in Table 11 (e.g., administering a metabolite that is associated with high or moderate physical activity).

TABLE 11
Metabolites Associates with Physical Activity

Cytosine
Glycosyl Ceramide (D18:2/24:1, D18:1/24:2)
Histidine
Docosapentaenoate (n6 DPA; 22:5n6)
Glycylvaline
Tyrosine
Proline
Methionine Sulfoxide
Hypoxanthine
Glycosyl-N-stearoyl-sphingosine (d18:1/18:0)
Thymine
Homocitrulline
Maltose
N-Acetyl-Beta-Glucosaminylamine
Ribulose/Xylulose
N-Acetyl-1-Methylhistidine
Serine
Leucine
Arachidoylcarnitine (C20)
2′-Deoxyguanosine
Phenylalanine
Aspartate
Lignoceroylcarnitine (C24)
Biocytin
Glycosyl-N-(2-Hydroxynervonoyl)-Sphingosine (D18:1/24:1(2Oh))
Stearoylcarnitine (C18)
Glycylisoleucine
Taurolithocholate
Threonine
Succinate
1-Methylnicotinamide
Argininate
Tyramine
Hyocholate

TABLE 12
Exemplary Metabolites

Cytosine
Histidine
Glycylvaline
Tyrosine
Proline
Hypoxanthine
Thymine
N-Acetyl-Beta-Glucosaminylamine
Ribulose/Xylulose
N-Acetyl-1-Methylhistidine
Serine
Leucine
Phenylalanine
Aspartate
Lignoceroylcarnitine (C24)
Glycylisoleucine
Succinate
Argininate
Tyramine
5alpha-androstan-3beta,17alpha-diol monosulfate
myo-inositol
5alpha-pregnan-3beta,20alpha-diol disulfate
5alpha-pregnan-3beta,20alpha-diol monosulfate
chenodeoxycholic acid sulfate
cholate sulfate
taurochenodeoxycholate
taurolithocholate 3-sulfate
hexadecatrienoate (16:3n3)
PAHSA (16:0/OH-18:0)
4-hydroxyphenylpyruvate
3-methyl-2-oxovalerate
4-methyl-2-oxopentanoate
beta-hydroxyisovalerate
2,3-dihydroxy-5-methylthio-4-pentenoate (DMTPA)
N-formylmethionine
hydroxy-N6,N6,N6-trimethyllysine
1-methyladenosine
cytidine
digalacturonic acid
7-methylurate
3,7-dimethylurate
3-(3-hydroxyphenyl)propionate
5-acetylamino-6-amino-3-methyluracil
nicotinamide riboside
xylose
succinylcarnitine
pentose acid

TABLE 13
Exemplary Metabolites

Cytosine
Glycosyl Ceramide (D18:2/24:1, D18:1/24:2)
Histidine
Glycylvaline
Tyrosine
Proline
Hypoxanthine
Thymine
Maltose
Ribulose/Xylulose
N-Acetyl-1-Methylhistidine
Serine
Leucine
Arachidoylcarnitine (C20)
Phenylalanine
Aspartate
Lignoceroylcarnitine (C24)
Biocytin
Glycylisoleucine
Taurolithocholate
Threonine
1-Methylnicotinamide
Argininate

Provided herein are methods of improving mood and/or ameliorating a depressive symptom, treating or preventing a psychiatric or neurologic disorder, and/or enhancing the beneficial effects of physical activity in a subject by administering an agent (e.g., any agent disclosed herein or known in the art, such as a small molecule or a peptide) to the subject that inhibits the activity of or lowers the levels of cytidine in the GI tract of the subject.

It should be appreciated that an agent disclosed herein, myoinositol, a metabolite and/or a bacteria disclosed herein can be administered conjointly to the subject.

In some embodiments, one or more compositions disclosed herein can be administered conjointly to the subject.

As used herein, the phrase “conjoint administration” refers to any form of administration of two or more different agents such that the second agent is administered while the previously administered agent is still effective in the body. For example, the compositions disclosed herein can be administered either in the same formulation or in a separate formulation, either concomitantly or sequentially.

In some embodiments, the method further comprises administering an antibiotic to the subject prior to administration of the agent, bacteria, or metabolite to the subject.

In some embodiments, the subject is showing a depressive symptom, such as anxiety, apathy, general discontent, guilt, hopelessness, loss of interest or pleasure in activities, mood swings, sadness, agitation, excessive crying, irritability, restlessness, social isolation, early awakening, excess sleepiness, insomnia, restless sleep, excessive hunger, fatigue, loss of appetite, lack of concentration, slowness in activity, suicidal ideation, weight gain and weight loss.

In some embodiments, the subject is afflicted with or at risk for a psychiatric or neurologic disorder, such as bipolar depression, post-partum depression (PPD), post-traumatic stress disorder (PTSD), major depressive disorder (MDD), or food addiction.

In some embodiment, the subject is at risk for or is afflicted with a psychiatric or neurologic disorder disclosed herein. The psychiatric or neurologic disorder may be depression, PPD, PTSD, MDD, food addiction, or bipolar disorder. In some embodiments, the subject has been diagnosed (e.g., by a clinician) prior to administration of a composition disclosed herein. In some embodiments, the subject is as risk for a psychiatric or neurologic disorder disclosed herein. As used herein, an “at risk” subject includes, for example, a subject with a family history of the psychiatric or neurologic disorder or a subject with other symptoms or disorders that are comorbid with the psychiatric or neurologic disorder.

In some embodiments, the methods further comprise administering an additional therapy for psychiatric or neurologic disorder, such as an antidepressant medication (e.g., citalopram, fluvoxamine, bupropion, escitalopram, sertralilm, mirtazapine, fluolsetine, venlafaxine, amitriptyline, or imipramine). In another aspect, the additional therapy is a cannabinoid, a stimulant, an anti-inflammatory agent, a steroid, a barbiturate, an opioid analgesic, a sleep agent (e.g. melatonin or eszopiclone), an anxiolytic, an antipsychotic, or a combination thereof.

The agents and compositions of the present disclosure can be used on combination with at least one medication or therapy useful, e.g., in treating or alleviating symptoms of a psychiatric or a neurological condition. Suitable examples of such medications include levodopa (L-dopa), carbidopa, safinamide, dopamine agonists (e.g., ropinirole, pramipexole, rotigotine), amantadine, trihexyphenidyl, benztropine, selegiline, rasagiline, tolcapone, and entacapone, or a pharmaceutically acceptable salt thereof. Other examples include antidepressants (e.g., SSRIs, SNRIs, or tricyclic antidepressants) and antipsychotics (e.g., aripiprazole, fluphenazine, haloperidol, paliperidone, or risperidone).

The compositions disclosed herein may be administered to a subject by any means known in the art, for example, the composition may be formulated for oral delivery. The composition may be in the form of a pill, tablet, or capsule. In some embodiments, the subject may be a mammal (e.g., a human). In some embodiments, the composition is self-administered.

It will be appreciated by a person of skill in the art the “at risk” in the following diagnostic methods may also include determining that the subject being screened is afflicted with a psychiatric or neurologic disorder disclosed herein. The methods may further comprise administration of a composition disclosed herein.

In some aspects, provided herein are methods for determining whether a subject is at risk for a psychiatric or neurologic disorder, the method comprising: obtaining a sample from the GI tract of the subject, optionally isolating microbial DNA from the sample, identifying the amount of at least one genus of bacteria selected from Megasphaera, Methanobrevibacter, Christensenellaceae, Prevotella, Fournierella, Acidaminococcus and Akkermansia (e.g., wherein the at least one genus of bacteria is selected from Megasphaera, Methanobrevibacter, Christensenellaceae, Prevotella, or Akkermansia) in the microbiome of the sample, and if the microbiome comprises a lower level of Megasphaera, Methanobrevibacter, Christensenellaceae, Prevotella, Fournierella, Acidaminococcus or Akkermansia (e.g., wherein the at least one genus of bacteria is selected from Megasphaera, Methanobrevibacter, Christensenellaceae, Prevotella, or Akkermansia) bacteria compared to a control level, the subject is considered at risk for the psychiatric or neurologic disorder.

In some embodiments, the control level is a level measured in a sample from the GI tract of the subject taken earlier in time. For example, if the sample taken earlier in time comprises at least 1%, 2%, 3%, 4%, 5%, 6%, 7%, 8%, 9%10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% more bacteria than a sample taken more recently (i.e., the level of the measured bacteria is decreasing over time), the subject is at risk or is afflicted with the psychiatric or neurologic disorder disclosed herein.

In other embodiments, the control level is a mean or median level of Megasphaera, Methanobrevibacter, Christensenellaceae, Prevotella, Fournierella, Acidaminococcus or Akkermansia bacteria in subjects not afflicted with or at risk for the psychiatric or neurologic disorder. For example, if the subject's sample has at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% less bacteria than a mean or median level of Megasphaera, Methanobrevibacter, Christensenellaceae, Prevotella, Fournierella, Acidaminococcus or Akkermansia in a subject population not afflicted with the psychiatric or neurologic disorder, the subject is determined to be at risk for or afflicted with the psychiatric or neurologic disorder.

In some aspects, provided herein are methods for determining whether a subject is at risk for a psychiatric or neurologic disorder, the method comprising: obtaining a sample from the GI tract of the subject, measuring the activity of or level of cytidine in the sample, and if the microbiome comprises high activity or a high level of cytidine compared to a control level, the subject is considered at risk for the psychiatric or neurologic disorder disclosed herein.

In some embodiments, the control level is a level measured in a sample from the GI tract of the subject taken earlier in time. For example, if the level of cytidine in the sample taken earlier in time comprises at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% less cytidine than a sample taken more recently (e.g., the level of cytidine is rising in the subject), the subject is at risk or is afflicted with the psychiatric or neurologic disorder disclosed herein.

In other embodiments, the control level is represented a mean or median measurement of the activity of or level of cytidine in the GI tract of the subjects not afflicted with or at risk for the psychiatric or neurologic disorder disclosed herein. For example, if the subject's sample has at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% more cytidine or cytidine activity than a mean or median level of cytidine or cytidine activity in a subject population not afflicted with the psychiatric or neurologic disorder, the subject is determined to be at risk for or afflicted with the psychiatric or neurologic disorder disclosed herein.

In some aspects, provided herein are methods for determining whether a subject is at risk for a psychiatric or neurologic disorder, the method comprising: obtaining a sample from the GI tract of the subject, measuring the level or activity of myoinositol in the sample, and if the sample comprises a lower level or activity of myoinositol compared to a control level, the subject is considered at risk for the psychiatric or neurologic disorder disclosed herein.

In some embodiments, the control level is a level measured in a sample from the GI tract of the subject taken earlier in time. For example, if the level of or activity of myoinositol in the sample taken earlier in time comprises at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% more myoinositol or myoinositol activity than a sample taken more recently (i.e., the level is lowering over time), the subject is at risk or is afflicted with the disorder.

In other embodiments, the control level is represented a mean or median level of the activity of or level of myoinositol in the GI tract of the subjects not afflicted with or at risk for the psychiatric or neurologic disorder disclosed herein. For example, if the subject's sample has at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% less myoinositol or myoinositol activity than the a mean or median level of myoinositol or myoinositol activity in a subject population not afflicted with the psychiatric or neurologic disorder, the subject is determined to be at risk for or afflicted with the psychiatric or neurologic disorder.

In some aspects, provided herein are methods for determining whether a subject is at risk for a psychiatric or neurologic disorder, the method comprising: obtaining a sample from the GI tract of the subject, measuring the level or activity of at least one of the metabolites listed in Table 12 or Table 13 in the sample, and if the sample comprises a lower level or activity of at least one of the metabolites listed in Table 12 or Table 13 compared to a control level, the subject is considered at risk for the psychiatric or neurologic disorder disclosed herein.

In some embodiments, the control level is a level measured in a sample from the GI tract of the subject taken earlier in time. For example, if the level of or activity of any one of the metabolites listed in Table 12 or Table 13 in the sample taken earlier in time comprises at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% more of such as metabolite or the activity thereof than a sample taken more recently (i.e., the level is lowering over time), the subject is at risk or is afflicted with the disorder.

In other embodiments, the control level is represented a mean or median level of the activity of or level of any one of the metabolites listed in Table 12 or Table 13 in the GI tract of the subjects not afflicted with or at risk for the psychiatric or neurologic disorder disclosed herein. For example, if the subject's sample has at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% less of a metabolite (or activity thereof) listed in Table 12 or Table 13 than the a mean or median level of such a metabolite (or activity thereof) in a subject population not afflicted with the psychiatric or neurologic disorder, the subject is determined to be at risk for or afflicted with the psychiatric or neurologic disorder.

In general, the above methods directly act to reduce the amount of pathogenic bacteria in a subject. In some embodiments, this includes any such therapy that achieves the same goal of reducing the number of pathogenic organisms, when used in combination with the composition described herein, would lead to replacement of the pathogenic microflora involved in the diseased state with natural microflora enriched in the gut not afflicted with a disease, or less pathogenic species occupying the same ecological niche as the type causing a disease state. For example, a subject may undergo treatment with antibiotics or a composition comprising antibiotics to target and decrease the prevalence of pathogenic organisms, and subsequently be treated with a composition described herein.

Suitable antimicrobial compounds include capreomycins, including capreomycin IA, capreomycin IB, capreomycin IIA and capreomycin IIB; carbomycins, including carbomycin A; carumonam; cefaclor, cefadroxil, cefamandole, cefatrizine, cefazedone, cefazolin, cefbuperazone, cefcapene pivoxil, cefclidin, cefdinir, cefditoren, cefime, ceftamet, cefmenoxime, cefmetzole, cefminox, cefodizime, cefonicid, cefoperazone, ceforanide, cefotaxime, cefotetan, cefotiam, cefoxitin, cefpimizole, cefpiramide, cefpirome, cefprozil, cefroxadine, cefsulodin, ceftazidime, cefteram, ceftezole, ceftibuten, ceftiofur, ceftizoxime, ceftriaxone, cefuroxime, cefuzonam, cephalexin, cephalogycin, cephaloridine, cephalosporin C, cephalothin, cephapirin, cephamycins, such as cephamycin C, cephradine, chlortetracycline; chlarithromycin, clindamycin, clometocillin, clomocycline, cloxacillin, cyclacillin, danofloxacin, demeclocyclin, destomycin A, dicloxacillin, dirithromycin, doxycyclin, epicillin, erythromycin A, ethanbutol, fenbenicillin, flomoxef, florfenicol, floxacillin, flumequine, fortimicin A, fortimicin B, forfomycin, foraltadone, fusidic acid, gentamycin, glyconiazide, guamecycline, hetacillin, idarubicin, imipenem, isepamicin, josamycin, kanamycin, leumycins such as leumycin A1, lincomycin, lomefloxacin, loracarbef, lymecycline, meropenam, metampicillin, methacycline, methicillin, mezlocillin, micronomicin, midecamycins such as midecamycin A1, mikamycin, minocycline, mitomycins such as mitomycin C, moxalactam, mupirocin, nafcillin, netilicin, norcardians such as norcardian A, oleandomycin, oxytetracycline, panipenam, pazufloxacin, penamecillin, penicillins such as penicillin G, penicillin N and penicillin O, penillic acid, pentylpenicillin, peplomycin, phenethicillin, pipacyclin, piperacilin, pirlimycin, pivampicillin, pivcefalexin, porfiromycin, propiallin, quinacillin, ribostamycin, rifabutin, rifamide, rifampin, rifamycin SV, rifapentine, rifaximin, ritipenem, rekitamycin, rolitetracycline, rosaramicin, roxithromycin, sancycline, sisomicin, sparfloxacin, spectinomycin, streptozocin, sulbenicillin, sultamicillin, talampicillin, teicoplanin, temocillin, tetracyclin, thostrepton, tiamulin, ticarcillin, tigemonam, tilmicosin, tobramycin, tropospectromycin, trovafloxacin, tylosin, and vancomycin, and analogs, derivatives, pharmaceutically acceptable salts, esters, prodrugs, and protected forms thereof.

Suitable anti-fungal compounds include ketoconazole, miconazole, fluconazole, clotrimazole, undecylenic acid, sertaconazole, terbinafine, butenafine, clioquinol, haloprogin, nystatin, naftifine, tolnaftate, ciclopirox, amphotericin B, or tea tree oil and analogs, derivatives, pharmaceutically acceptable salts, esters, prodrugs, and protected forms thereof.

Suitable antiviral agents include acyclovir, azidouridine, anismoycin, amantadine, bromovinyldeoxusidine, chlorovinyldeoxusidine, cytarabine, delavirdine, didanosine, deoxynojirimycin, dideoxycytidine, dideoxyinosine, dideoxynucleoside, desciclovir, deoxyacyclovir, efavirenz, enviroxime, fiacitabine, foscamet, fialuridine, fluorothymidine, floxuridine, ganciclovir, hypericin, idoxuridine, interferon, interleukin, isethionate, nevirapine, pentamidine, ribavirin, rimantadine, stavudine, sargramostin, suramin, trichosanthin, tribromothymidine, trichlorothymidine, trifluorothymidine, trisodium phosphomonoformate, vidarabine, zidoviridine, zalcitabine and 3-azido-3-deoxythymidine and analogs, derivatives, pharmaceutically acceptable salts, esters, prodrugs, and protected forms thereof.

Other suitable antiviral agents include 2′,3′-dideoxyadenosine (ddA), 2′,3′-dideoxyguanosine (ddG), 2′,3′-dideoxycytidine (ddC), 2′,3′-dideoxythymidine (ddT), 2′3′-dideoxy-dideoxythymidine (d4T), 2′-deoxy-3′-thia-cytosine (3TC or lamivudime), 2′,3′-dideoxy-2′-fluoroadenosine, 2′,3′-dideoxy-2′-fluoroinosine, 2′,3′-dideoxy-2′-fluorothymidine, 2′,3′-dideoxy-2′-fluorocytosine, 2′3′-dideoxy-2′,3′-didehydro-2′-fluorothymidine (Fd4T), 2′3′-dideoxy-2′-beta-fluoroadenosine (F-ddA), 2′3′-dideoxy-2′-beta-fluoro-inosine (F-ddI), and 2′,3′-dideoxy-2′-beta-flurocytosine (F-ddC). In some embodiments, the antiviral agent is selected from trisodium phosphomonoformate, ganciclovir, trifluorothymidine, acyclovir, 3′-azido-3′-thymidine (AZT), dideoxyinosine (ddI), and idoxuridine and analogs, derivatives, pharmaceutically acceptable salts, esters, prodrugs, and protected forms thereof.

### Pharmaceutical Compositions

In some aspects, the invention relates to a composition (e.g., a pharmaceutical composition) comprising a bacteria, a metabolite, and/or an agent disclosed herein.

The pharmaceutical compositions disclosed herein may be delivered by any suitable route of administration, including orally, buccally, sublingually, parenterally, and topically, as by powders, ointments, drops, liquids, gels, or creams. In certain embodiments, the pharmaceutical compositions are delivered generally (e.g., via oral or parenteral administration). In certain other embodiments, the pharmaceutical compositions are delivered locally through injection.

The present application also provides pharmaceutical compositions comprising an effective amount of a compound of the present disclosure disclosed herein, or a pharmaceutically acceptable salt thereof; and a pharmaceutically acceptable carrier. The pharmaceutical composition may also comprise any one of the additional therapeutic agents described herein. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

In certain embodiments, a provided composition comprises one or more disintegrants or solubilizing agents, such as a cyclodextrin, or a carboxymethyl cellulose, calcium carboxymethyl cellulose, low-substituted hydroxypropyl cellulose, sodium croscarmellose, sodium carboxy starch, calcium carbonate, sodium carbonate and the like.

Actual dosage levels of the active ingredients in the pharmaceutical compositions may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the compounds employed in the pharmaceutical composition at levels lower than that required in order to achieve the desired therapeutic effect and gradually increase the dosage until the desired effect is achieved.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant. The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

### Nutraceutical Composition

A nutraceutical composition is a pharmaceutical alternative which may have physiological benefits. In some embodiments, a nutraceutical composition is a food (or part of a food) that provides medical or health benefits, including the prevention and/or treatment of a disease. See, e.g., Brower (1998) Nat. Biotechnol. 16:728-731; Kalra (2003) AAPS Pharm Sci. 5(3):25. In other embodiments, a nutraceutical composition is a dietary or nutritional supplement. The compositions described herein may be a nutraceutical composition.

Accordingly, a nutraceutical composition of the invention can be a food product, foodstuff, functional food, or a supplement composition for a food product or a foodstuff. As used herein, the term food product refers to any food or feed which provides a nutritional source and is suitable for oral consumption by humans or animals. The food product may be a prepared and packaged food (e.g., mayonnaise, salad dressing, bread, or cheese food) or an animal feed (e.g., extruded and pelleted animal feed, coarse mixed feed or pet food composition). As used herein, the term foodstuff refers to a nutritional source for human or animal oral consumption. Functional foods refer to foods being consumed as part of a usual diet but are demonstrated to have physiological benefits and/or reduce the risk of chronic disease beyond basic nutritional functions.

Food products, foodstuffs, functional foods, or dietary supplements may be beverages such as non-alcoholic and alcoholic drinks as well as liquid preparations to be added to drinking water and liquid food. Non-alcoholic drinks are for instance soft drinks; sport drinks; fruit juices, such as orange juice, apple juice and grapefruit juice; lemonades; teas; near-water drinks; and milk and other dairy drinks such as yogurt drinks, and diet drinks. In other embodiments, food products, foodstuffs, functional foods, or dietary supplements refer to solid or semi-solid foods. These forms can include, but are not limited to, baked goods such as cakes and cookies; puddings; dairy products; confections; snack foods (e.g., chips); or frozen confections or novelties (e.g., ice cream, milk shakes); prepared frozen meals; candy; liquid food such as soups; spreads; sauces; salad dressings; prepared meat products; cheese; yogurt and any other fat or oil containing foods; and food ingredients (e.g., wheat flour). In some embodiments, the food products, foodstuffs, functional foods, or dietary supplements may be in the form of tablets, boluses, powders, granules, pastes, pills or capsules for the ease of ingestion.

It is understood by those of skill in the art that in additional to isolated, and optionally purified and/or sonicated compositions of the present disclosure and other ingredients can be added to food products, foodstuffs, or functional foods described herein, for example, fillers, emulsifiers, preservatives, etc. for the processing or manufacture of the same. Additionally, flavors, coloring agents, spices, nuts and the like may be incorporated into the nutraceutical composition. Flavorings can be in the form of flavored extracts, volatile oils, chocolate flavorings, peanut butter flavoring, cookie crumbs, crisp rice, vanilla or any commercially available flavoring.

Emulsifiers can also be added for stability of the nutraceutical compositions. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product. Preservatives can also be added to the nutritional supplement to extend product shelf life. Preferably, preservatives such as potassium sorbate, sodium sorbate, potassium benzoate, sodium benzoate or calcium disodium EDTA are used.

In addition, the nutraceutical composition can contain natural or artificial (preferably low calorie) sweeteners, e.g., saccharides, cyclamates, aspartamine, aspartame, acesulfame K, and/or sorbitol. Such artificial sweeteners can be desirable if the nutraceutical composition is intended to be consumed by an overweight or obese individual, or an individual with type II diabetes who is prone to hyperglycemia.

Moreover, a multi-vitamin and mineral supplement can be added to the nutraceutical compositions of the present invention to obtain an adequate amount of an essential nutrient, which is missing in some diets. The multi-vitamin and mineral supplement can also be useful for disease prevention and protection against nutritional losses and deficiencies due to lifestyle patterns.

Accordingly, particular embodiments of the invention provide for the nutritional source of the nutraceutical to modulate endogenous commensal bacterial populations. Such modulation can be achieved by modification of gut pH, consumption of beneficial bacteria (e.g., as in yogurt), by providing nutritional sources (e.g., prebiotics) that select for particular populations of bacteria, or by providing antibacterial compounds. Such modulation can mean an increase or decrease in the gut microbiota populations or ratios. In particular embodiments, the absolute or relative numbers of desirable gut microorganisms is increased and/or the absolute or relative numbers of undesirable gut microorganisms is decreased. For example, it is contemplated that there are a variety of nutritional sources exhibiting antibacterial activity that can be used to modulate gut microbiota populations. For example, garlic has been shown to produce the compound allicin (allyl 2-propenethiosulfinate), which exhibits antibacterial activity toward E. coli (Fujisawa, et al. (2009) Biosci. Biotechnol. Biochem. 73 (9):1948-55; Fujisawa, et al. (2008) J. Agric. Food Chem. 56(11):4229-35). Similarly, rosemary extracts and other essential oils have been shown to contain antibacterial activity (Klancnik, et al. (2009) J. Food Prot. 72(8):1744-52; Si, et al. (2006) J. Appl. Microbiol. 100(2):296-305). Extracts of the edible basidiomycete, Lentinus edodes (Shiitake), have also been shown to possess antibiotic activity (Soboleva, et al. (2006) Antibiot. Khimioter. 51(7):3-8; Hirasawa, et al. (1999) Int. J. Antimicrob. Agents 11(2):151-7). Moreover, purple and red vegetable and fruit juices exhibit antibacterial activities (Lee, et al. (2003) Nutrition 19:994-996).

Furthermore, it is contemplated herein that the food products, foodstuffs, functional foods, or dietary supplements may be combined with antibiotics to control the gut microbiota populations.

The nutraceutical composition of the present invention can be provided in a commercial package, alone, or with additional components, e.g., other food products, food stuffs, functional foods, dietary supplement. Desirably, the commercial package has instructions for consumption of the instant nutraceutical, including preparation and frequency of consumption, and use in the prevention or treatment of inflammatory diseases, autoimmune diseases and cancer. Moreover, in particular embodiments, the commercial package further includes a natural product (e.g., the food, extracts, antibiotics, and oils) that modulates endogenous commensal bacterial populations. A package containing both a nutraceutical of the present disclosure in combination with said natural product can contain instructions for consuming the natural product, e.g., in advance (e.g., 2, 4, 6 or 8 or more hours) of consuming the nutraceutical in order to enhance the activity of the nutraceutical composition.

### EXEMPLIFICATION

### Example 1: Improved Psychosocial Measures Associated with Physical Activity is Explained by Alterations in Brain-Gut Microbiome Signatures; Identification of Agents To Treat Disorders Disclose Herein

Obesity contributes to physical comorbidities and mental health consequences. It is explored herein whether physical activity can influence more than metabolic regulation and result in psychological benefits through the brain-gut microbiome (BGM) system in an obese population. Fecal samples were obtained for 16s rRNA profiling and fecal metabolomics, along with psychological and physical activity questionnaires. Whole brain resting-state functional MRI was acquired, and brain connectivity metrics were calculated. High physical activity was significantly associated with increased connectivity in appetite control brain regions, while low physical activity increased emotional regulation connections. High physical activity was associated with mental and obesity-related microbiome (Megasphaera, Methanobrevibacter, Akkermansia, Christensenellaceae, Prevotella) and metabolite (myoinositol) signatures. Greater resilience and coping, and lower levels of food addiction seen with higher physical activity, may be explained by differences seen in the BGM system. These novel findings provide an emphasis on the psychological benefits of physical activity, beyond metabolic regulation.

### Introduction

In response to weight discrimination, modifiable factors such as resilience may serve a protective function against the negative psychological and physical consequences of obesity-related stress⁷. Resilience is defined as the ability to positively adapt in response to significant adversity or stressors, and develops via interplay between genetics, environmental factors, and social support systems⁸. Studies have shown that emotional resilience is protective against the development of obesity regardless of income, through positive associations with healthier dietary choices and moderating perceived stress and binge eating behavior^{9,10}. An additional finding in a study with adolescents exploring the relationship between weight status and resilience was that greater resilience was found to be associated with physical activity¹¹. In adults, physical activity is a well-recognized contributor to psychological resilience by blunting stress reactivity, protecting against the metabolic consequences of stress-inducing events, and promoting an anti-inflammatory state¹²⁻¹⁴ While some studies have been done on the individual physiological changes associated with physical activity in obese populations, there are a limited number of studies on the interactions between physical activity and various psychological variables in the context of the brain-gut microbiome (BGM) axis.

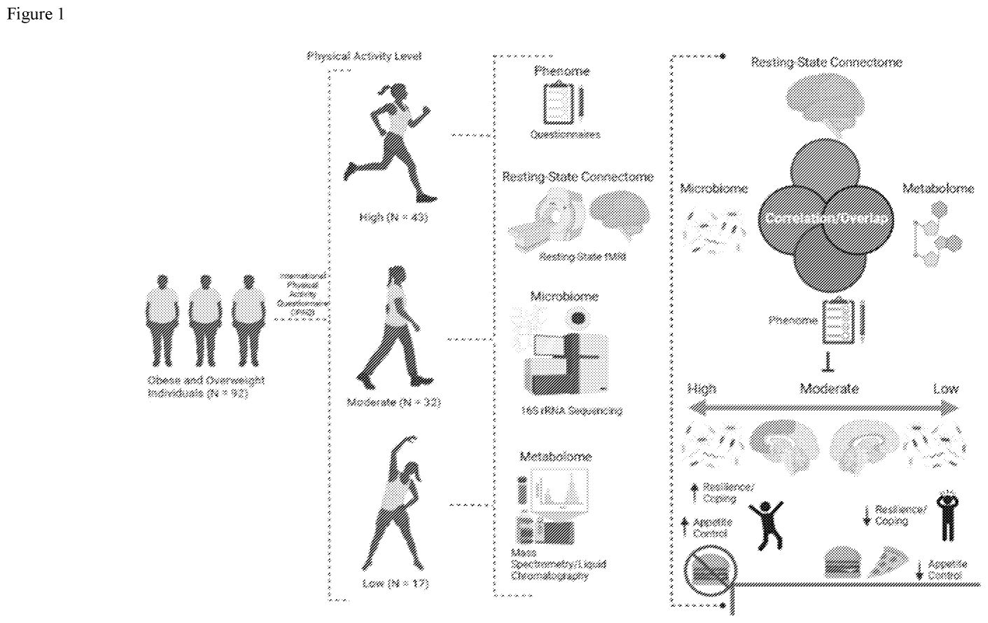

A growing body of preclinical and clinical studies support the role of the BGM axis in the pathophysiology of obesity, mediated by alterations in metabolic, enteroendocrine, and neural signaling¹⁵. Obesity has been associated with changes in the diversity and composition of the gut microbiota, which leads to disruptions in the downstream microbially derived metabolites and gut-endocrine signals that orchestrate energy homeostasis¹⁶. For example, a recent study found obesity to be associated with increases in the Prevotella/Bacteroides ratio and corresponding decreases in fecal tryptophan levels, which is a metabolite directly related to the biosynthesis of important neurotransmitters such as serotonin¹⁷. Signals from the microbiome may thus also alter neural processes, with functional neuroimaging studies in obese individuals demonstrating alterations in reward and emotional regulation brain regions, which have also been linked to clinical measures such as food addiction, a state of hedonic-driven eating behavior involving continued consumption of palatable foods despite meeting homeostatic energy requirements.^{18,19}. While the effects of physical activity on the brain, microbiome, and metabolites may have been examined independently, there is a lack of studies that utilize a systems-biology approach to study the effects of physical activity within the BGM as an integrated system, while incorporating clinical variables in the context of obesity. In this study, shows that there are distinct brain, gut microbiome, and metabolite signatures based on physical activity, and that these BGM system differences modulate positive psychological changes in an obese population (summarized in FIG. 1).

### Methods

### Participants

The sample was comprised of 92 right-handed participants, with the absence of significant medical or psychiatric conditions. Participants were excluded for the following: pregnant or lactating, substance use, abdominal surgery, tobacco dependence (half a pack or more daily), extreme strenuous exercise (>8 h of continuous exercise per week), current or past psychiatric illness and major medical or neurological conditions. Participants taking medications that interfere with the central nervous system or regular use of analgesic drugs were excluded. Because of the effect of handedness on fMRI activation, only right-handed participants were included to negate handedness as a cofounder. Included participants were also required to not have taken antibiotics or probiotics for at least 3 months before enrolling in the study. Only premenopausal females were enrolled and were scanned during the follicular phase of their menstrual cycles as determined by self-report of their last day of the cycle. Participants with hypertension, diabetes, metabolic syndrome or eating disorders were excluded to minimize confounding effects. We used body mass index (BMI) cutoffs to define our overweight (25≤BMI≤30) and obese (BMI≥30) groups. No participants exceeded 400 lbs due to magnetic resonance imaging (MRI) scanning weight limits. Participants underwent MRI scans, anthropometrics (height, body weight, and body mass index), and fresh stool samples for 16s ribosomal RNA gene sequencing and metabolite analysis were collected.

All procedures complied with and were approved by the Institutional Review Board (16-000187, 16-000281) at the University of California, Los Angeles's Office of Protection for Research Subjects. All participants provided written informed consent. FIG. 1 summarizes the workflow for data collection and data analyses. Additional details on the questionnaires used as well as the processing and analysis of the microbiome, metabolite, and brain data are detailed in the supplemental methods section below.

### Statistical Analyses

The student's test was used for continuous variables and chi-squared test for categorical variables when analyzing baseline demographic and behavioral differences. Means were reported with their corresponding standard deviations.

We calculated beta diversity using DEICODE plugin in QIIME 2, which accounts for sparse compositional nature of microbiome data with a robust Aitchison analysis. This method has been shown to yield higher discriminatory power compared to other common metrics, such as UniFrac or Bray-Curtis.(42) Alpha diversity was calculated in QIIME using OTU-level data rarefied to 32,303 sequences and significance was determined using Faith's phylogenetic diversity (Faith's PD), Chaol, and Shannon index by analysis of variance. Association of microbial genera were evaluated using DESeq2 in R, which uses an empirical Bayesian approach to minimize dispersion and fit non-rarified count data to a negative binomial model. Differential abundance p-values were converted to q-values to adjust for multiple hypothesis testing (<0.05 for significance).

Sparse partial least squares discriminant analysis (sPLS-DA) was conducted using the R package mixOmics as a data reduction method for the resting-state brain connectivity and metabolites separately as previously described^{20,21}. The data was normalized during preprocessing before being entered into sPLS-DA models. The values from the sPLS-DA components were assessed by using a generalized linear model (GLM) in R, controlling for age, sex, BMI, and diet. Only FDR-corrected p values, which are referred to as q-values, are reported. Only results surviving FDR correction were reported.

For integrated analyses, significant findings from fMRI, metabolite, 16S microbiome, and clinical data were combined into one dataset, and spearman correlations between datapoints were performed using the Hmisc and corrplot packages in R. All p-values were adjusted for multiple hypothesis testing using false discovery rate correction. Correlation networks were then visualized using Circos plots²².

### Results

### Participant Characteristics, Clinical Variables, and Psychosocial Measures

Clinical and behavioral characteristics of the 92 overweight and obese individuals (mean BMI=33.22 kh/m², mean age=32.84 years) are summarized in Table 1. Based on the IPAQ scoring guidelines for determining physical activity levels, the average total physical activity in the high (n=43), moderate (n=32), and low (n=17) groups were 13,432.84 METs, 1,822.953 METs, and 5,081.70 METs respectively (p<0.001). There were no differences in bioimpedance analysis or average BMIs between the groups. Education level was highest in the low physical activity group and was the lowest in the high physical activity group.

The high physical activity group had greater average BRS resilience scores (p=0.04) and ability to cope through acceptance of reality (p=0.04), and a significant difference compared to the low physical activity group. A trend in the anxiety scores were also seen with the HAD and STAI measures, with highest anxiety scores seen in the low physical activity groups, even though these did not reach significance.

Based on physical activity, there were also significant differences in multiple food addiction measures, as assessed using the Yale Food Addiction Scale (YFAS), with food craving scores being lowest with high physical activity group. When comparing between high vs. low physical activity groups, significant differences were found with the following YFAS measures: continued use (p=0.025), giving up (p=0.005), time spent (p<0.001), loss of control (p=0.01), and symptom count (p<0.001). Significant differences were also seen between moderate vs. low physical activity for the following YFAS measures: tolerance (p=0.04), continued use (p=0.04), time spent (p=0.002), loss of control (p=0.02), and symptom count (p=0.003; Table 1).

### Physical Activity Differentiates Brain Functional Connectivity

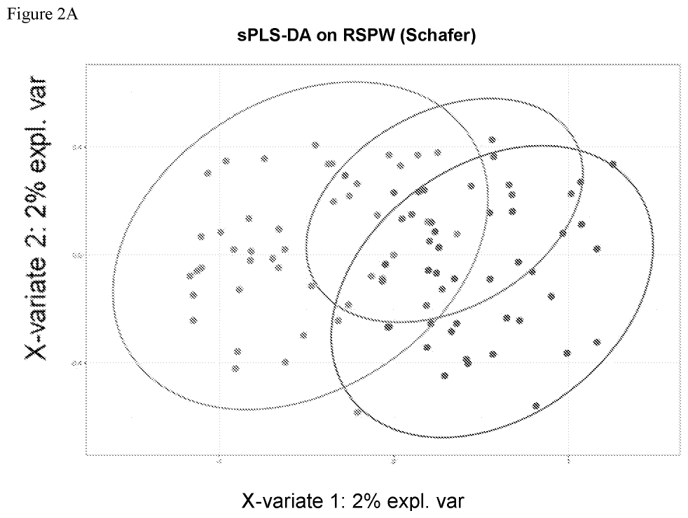

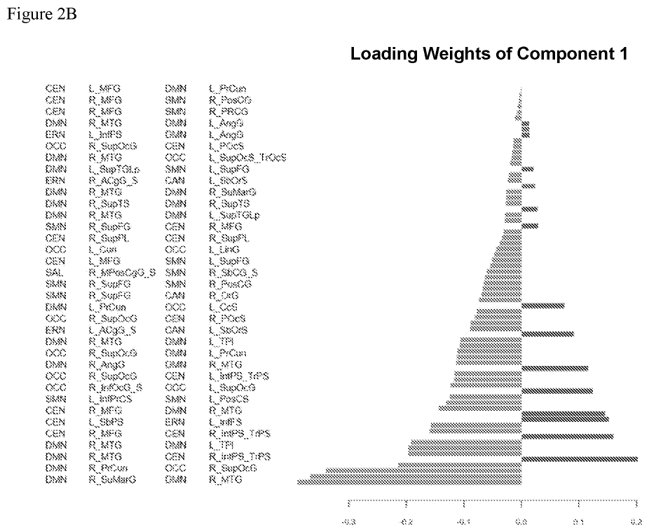

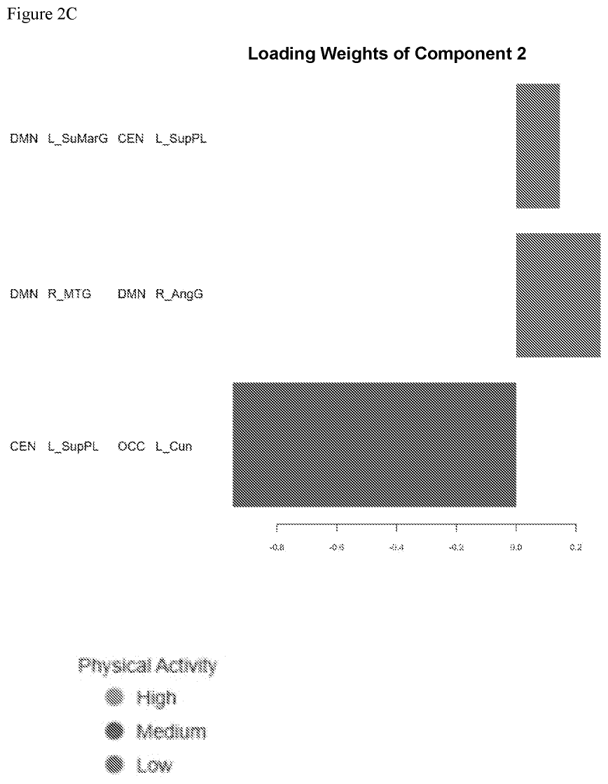

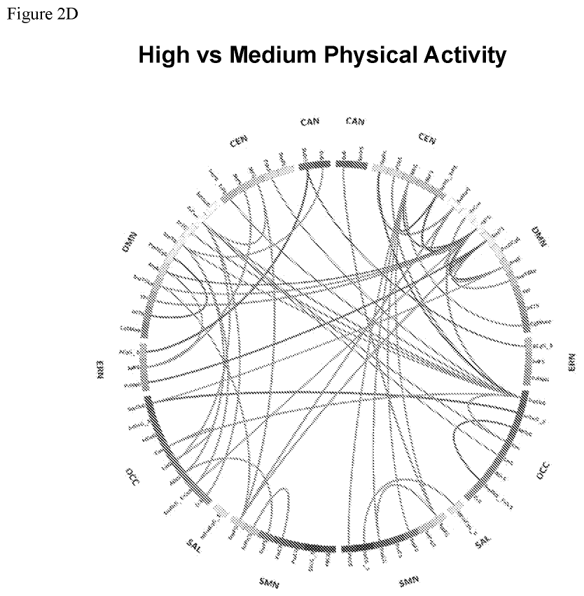

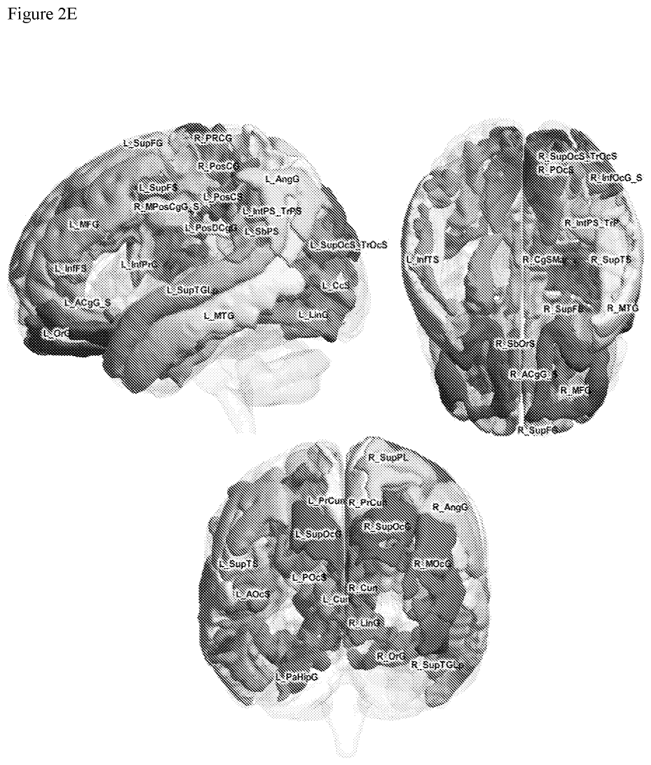

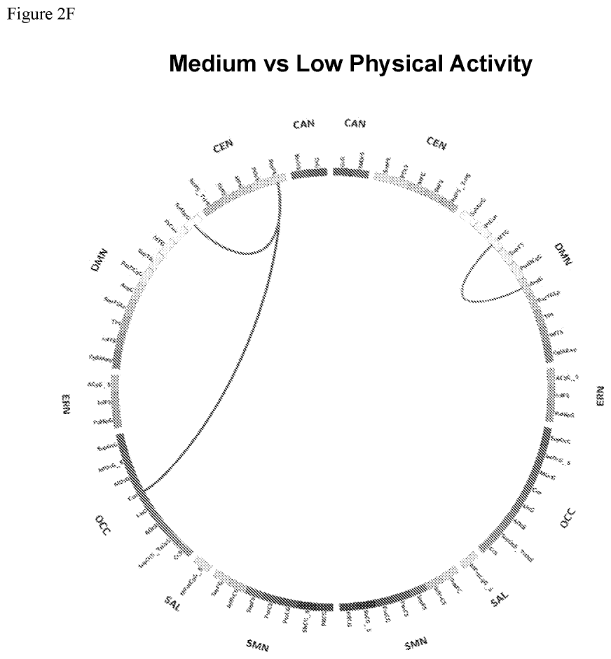

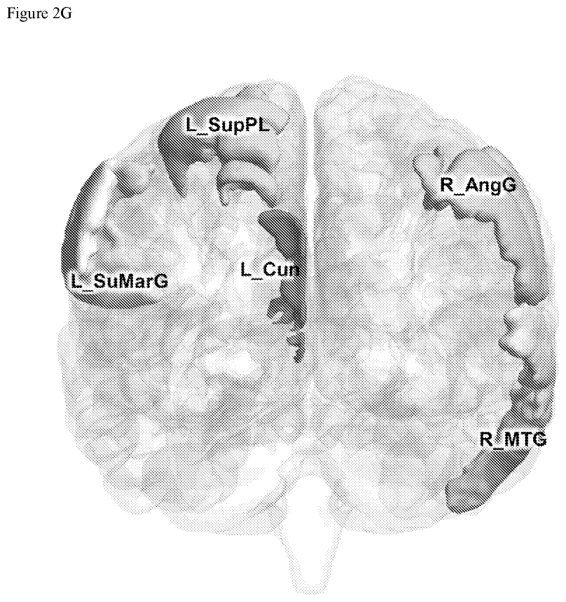

A sPLS-DA of brain functional connectivity displayed significant clustering based on physical activity level (FIG. 2A). Connectivity between 73 pairs of brain regions were significantly associated with physical activity (p<0.05), and after correcting for multiple comparisons 67 pairs and 3 pairs of regions remained significant (q<0.05) for the high vs. moderate physical activity and moderate vs. low physical activity groups respectively. The brain networks involved included the salience (SAL), central autonomic²³, central executive²⁴, emotional regulation (ERN), sensorimotor (SMN), default mode (DMN), and occipital²⁵ networks (specific brain regions summarized in Table 2).

Compared to moderate physical activity individuals, those with high physical activity have significantly increased functional connectivity in 52 pairs of brain connections, involving the default mode network (DMN), central executive network (CEN), sensorimotor network (SMN), occipital network (OCC), central autonomic network (CAN), emotional regulation network (ERN(, and salience network (SAL) (q<0.05). In contrast to high physical activity participants, those with moderate physical activity had significantly increased functional connectivity in 15 pairs of brain regions, and including brain regions involving the DMN, CEN, OCC, ERN, and CAN networks (q<0.05) (FIGS. 2B, 2C, 2D, 2E, Table 2).

When comparing moderate versus low physical activity, there were 3 pairs of connections that were significantly different (q<0.05) in functional connectivity involving the networks CEN, DMN, and OCC. Moderate physical activity individuals had increased connectivity between the left superior parietal lobule and left cuneus compared to low physical activity individuals. However, low physical activity individuals had increased functional connectivity between the right middle temporal gyrus and angular gyrus as well as left supramarginal gyrus and superior parietal lobule ((FIGS. 2B, 2C, 2F, 2G Table 2).

### Physical Activity and Gut Microbiome Composition

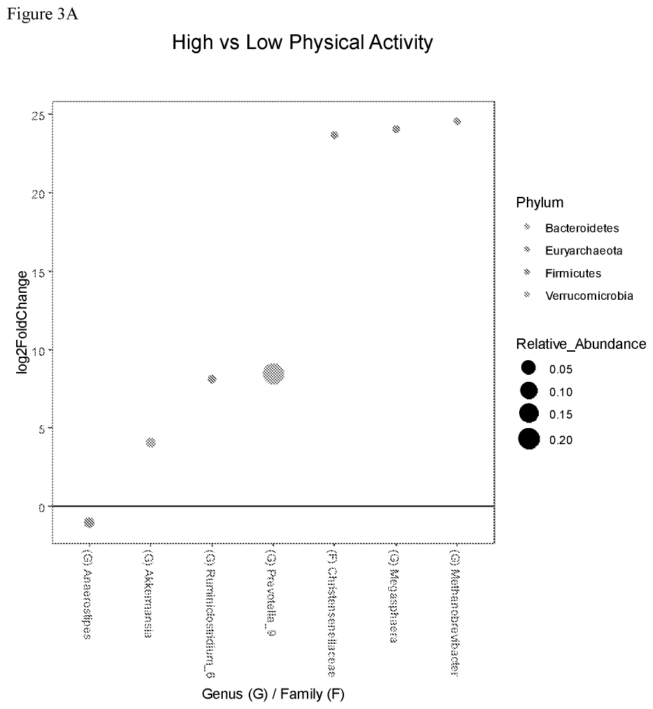

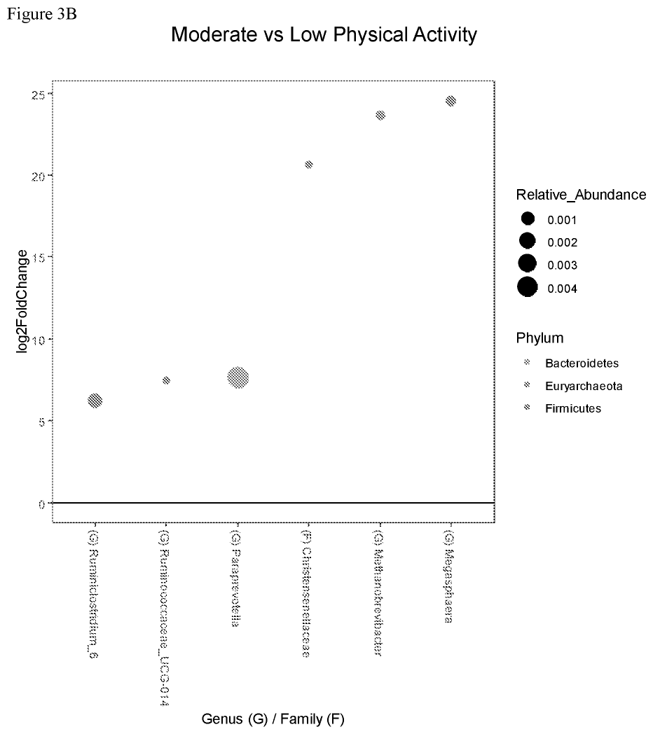

There were no significant relationships between the microbial samples based on physical activity for alpha or beta diversity metrics. DESEq2 analysis comparing the three physical activity groups identified distinct OTUs that were correlated with physical activity levels (Table 3, FIG. 3). When comparing high and low physical activity participants, 6 OTUs were positively associated and 1 OTU (Anaerostipes) was negatively associated with high physical activity. Methanobrevibacter demonstrated the greatest positive fold change with high physical activity. The largest abundant OTU belonged to the genera Prevotella. The four OTUs that increased in relative abundance with more physical activity were Methanobrevibacter, Megasphaera, Christensenellaceae, Ruminiclostridium, with positive fold changes seen when comparing both high versus low and moderate versus low physical activity (Table 3, FIG. 3A). The other two OTUs that demonstrated positive fold changes with moderate versus low physical activity were Ruminococcaceae and Paraprevotella, which represented the largest abundant OTU in this group comparison (Table 3, FIG. 3B).

Fecal Metabolites Associated with Physical Activity

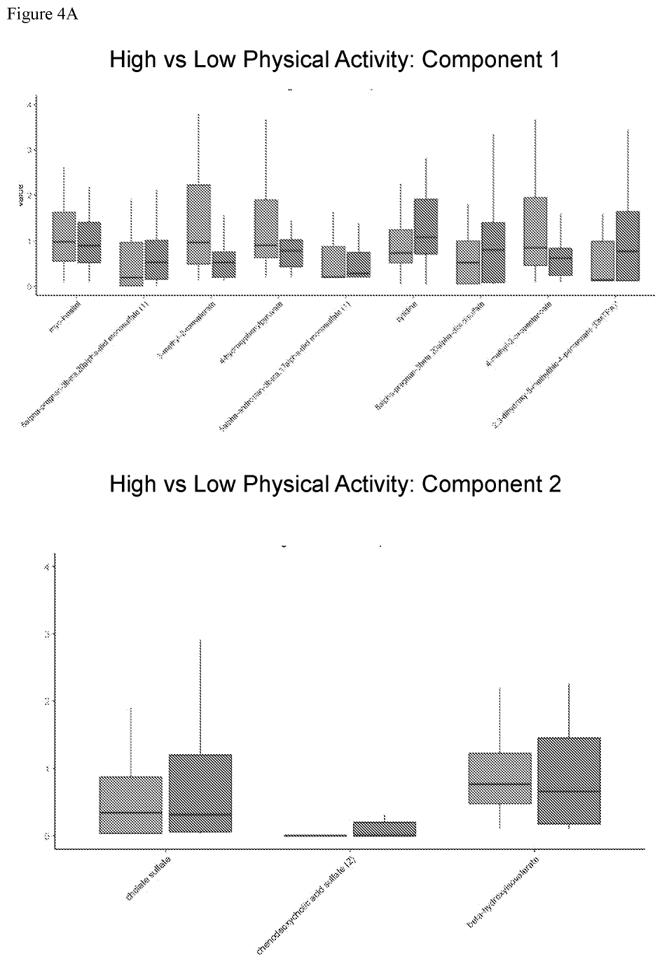

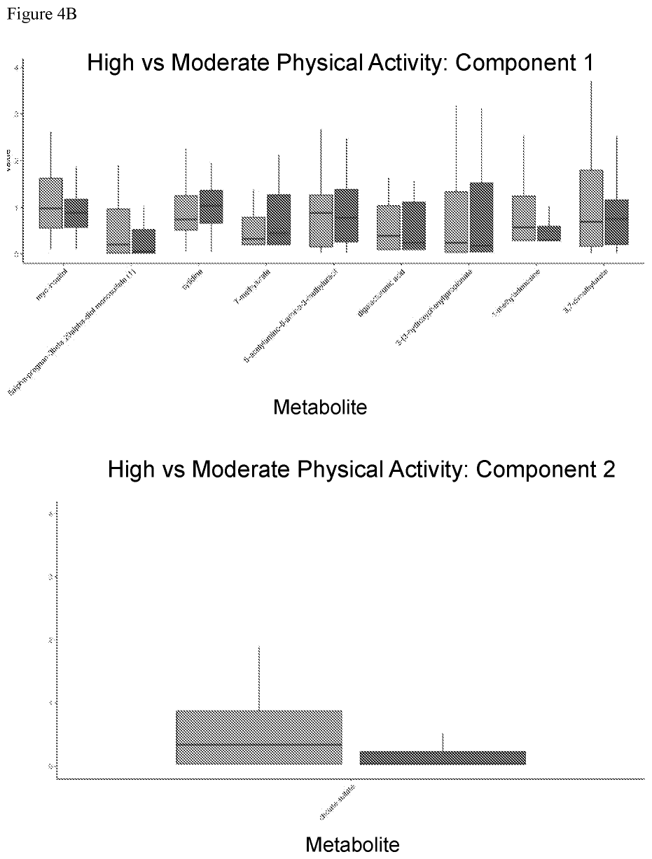

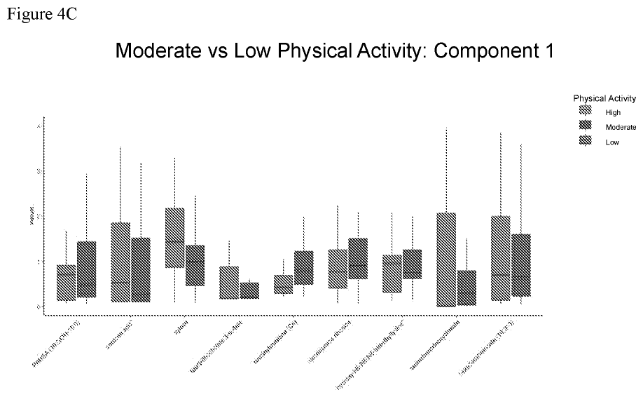

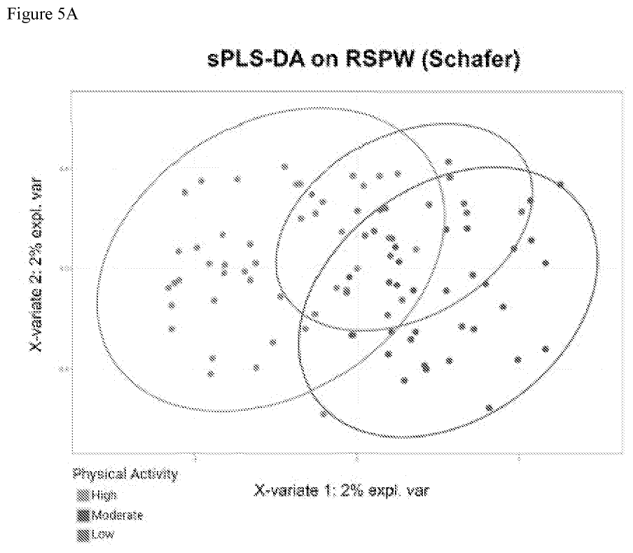

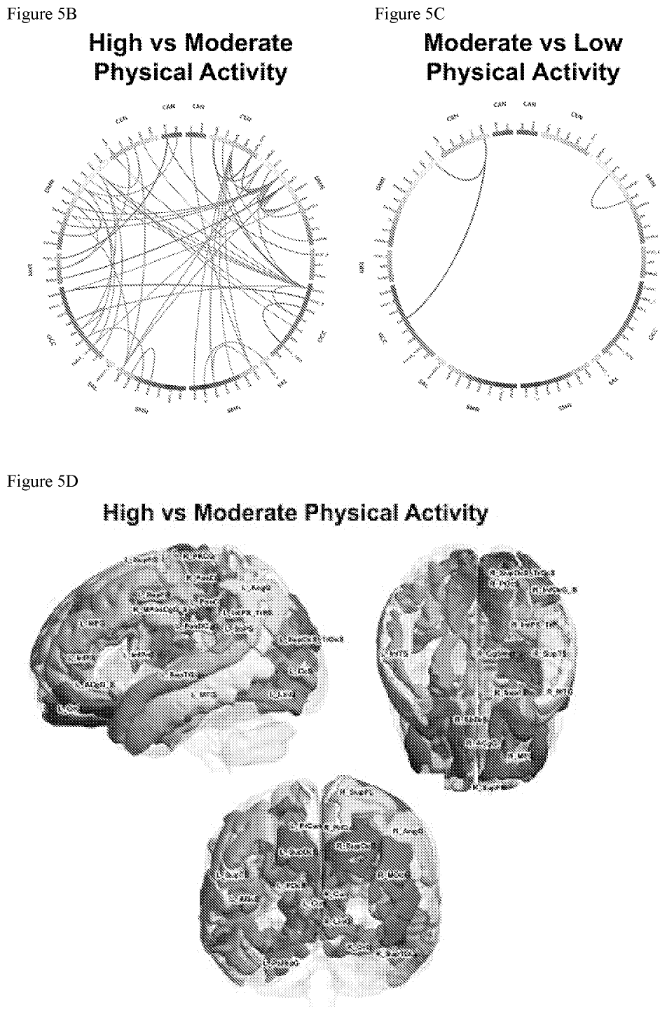

27 fecal metabolites were significantly correlated with physical activity level and belonged to the following super-pathways: lipid (10 metabolites), amino acid (6), xenobiotics (5), nucleotide (2), cofactors/vitamins (1), carbohydrates (1), energy (1), and partially characterized molecules (1) (Table 4, FIG. 4).

The high physical activity group displayed higher levels of myoinositol and 5alpha-androstan-3beta,17alpha-diol monosulfate were lipid metabolites when compared to both low (q=0.03 and q=0.03) and moderate physical activity (q=0.05 and q=0.02) (Table 4, FIG. 4A, 4B). Taurochenodeoxycholate was another metabolite belonging to the lipid super-pathway that was elevated with more physical activity when comparing moderate versus low physical activity (q=0.03) (Table 4, FIG. 4C). The other 7 lipid metabolites were seen to be increased with less physical activity when comparing either low versus moderate, moderate versus high, or low versus high (Table 4, FIG. 4A, 4B, 4C).

Within the 7 significant metabolites belonging to the amino acid super-pathways, 4-hydroxyphenylpyruvate (q=0.03), 3-methyl-2-oxovalerate (q=0.03), and 4-methyl-2-oxopentanoate (q=0.04) were increased in high physical activity versus low physical activity participants (FIG. 4A), while 2,3-dihydroxy-5-methylthio-4-pentenoate (DMTPA) (q=0.05), N-formylmethionine (q=0.05), and hydroxy-N6,N6,N6-trimethyllysine (q=0.03) were elevated with low physical activity versus either high or moderate physical activity participants (FIG. 4A, 4C).

The metabolites belonging to the xenobiotics super-pathway were seen to be either significantly increased or decreased with physical activity. Digalacturonic acid (q=0.05), 7-methylurate (q=0.040), and 3,7-dimethylurate (q=0.05) were significantly increased with more physical activity, specifically when comparing high versus moderate physical activity (FIG. 4B). 5-acetylamino-6-amino-3-methyluracil was seen to be highest in moderate physical activity participants when compared to both high (q=0.04) and low (q=0.01) physical activity individuals (FIG. 4B, 4C). 3-(3-hydroxyphenyl)propionate was also significantly increased in moderate physical activity when compared to high physical activity (1=0.05) (FIG. 4B).

Physical Activity Correlates with Alterations in the Brain-Gut-Microbiome Axis and Psychosocial Measures

Significant associations were identified between psychosocial variables (coping, resilience score, food addiction measures, education), metabolites (5alpha-Androstane-3beta,17alpha-diol monosulfate; myoinositol; cytidine; nicotinamide riboside; 5-Acetylamino-6-amino-3-methyluracil), and pairs of connected brain regions across all physical activity group comparisons.

In the high versus low physical activity comparison, significant associations were found between psychosocial variables and with a microbial genera and fecal metabolite, specifically between a measure of coping (acceptance) and Megasphaera (q=0.02) as well as BRS resilience score and cytidine (q=0.01). Associations between four pairs of fecal metabolites and genera were also found: cytidine and Methanobrevibacter, 5alpha-Androstane-3beta,17alpha-diol monosulfate 1 and Methanobrevibactor, 5alpha-Androstane-3beta,17alpha-diol monosulfate land Ruminiclostridium 6, as well as myoinositol and Akkermansia.

In the moderate versus low physical activity comparison, significant associations between two pairs of brain connections and with food addiction measures, microbial genus, and fecal metabolites were found. Increased functional connectivity between the right middle temporal gyrus and angular gyrus was associated with four YFAS food addiction measures (tolerance (q=0.05), continued use (q=0.04), loss of control (q=0.04), and symptom count (q=0.04)), 2 microbial genera (Methanobrevibactor. (q=0.03), Ruminococcaceae (q=0.05)), and with five metabolites (xylose (q=0.04), succinylcarnitine (q=0.05), pentose acid (q<0.001), taurolithocholic acid 3-sulfate (q=0.01), 5-acetylamino-6-amino-3-methyluracil (AAMU) (q=0.04)). Increased connectivity between the left superior parietal lobe and cuneus was associated with YFAS (time spent) (q=0.03) and the genera Ruminococcaceae (q=0.05). The YFAS food addictions measures were also associated with the microbiome and variables metabolites, specifically YFAS (loss control) with Methanobrevibactor (q=0.05), YFAS (tolerance and given up) with nicotinamide riboside (q=0.03), and YFAS given up, time spent, and loss of control with AAMU (q<0.001, q=0.05, q=0.01 respectively). Three genera were also associated with four metabolites, specifically Megasphaera with taurochenodeoxycholate (q<0.001) and xylose (q=0.05), Ruminoclostridium 6 with xylose (q=0.04), and Ruminococcaceae with succinylcarnitine (q=0.05) and pentose acid (q=0.04).

Within the high versus moderate physical activity participants, education level was significantly associated with 10 pairs of brain connections involving the DMN, CEN, ERN, OCC, SMN, and CAN networks as well as the metabolite hydroxyphenyl propionate (q=0.03). There were also 29 pairs of brain connections that were correlated to one of 12 metabolites.

### Discussion

There are few mechanistic studies that utilize an integrative approach to understand how physical activity may promote mental health and even fewer that incorporate the brain-gut microbiome system.

In this study, we demonstrated that there are significant alterations associated with physical activity seen in the functional connectivity of the brain, relative abundance of the gut microbiome, and thus the metabolites produced, and these BGM system alterations are associated with improved psychosocial measures in an overweight and obese population. Given that obese individuals face additional weight-related stressors compared to normal-weight healthy individuals, these findings highlight the utility of physical activity in preventing and treating mental illnesses in the obese population and how physical activity promotes health beyond just metabolic regulation.

In this study, an association was identified between higher physical activity and greater resilience, which is a known protective factor against the development of psychiatric disorders such as depression and post-traumatic stress disorder (PTSD). Interestingly, our analyses indicated that greater resilience within the high physical activity group was also associated with lower cytidine levels. Cytidine has been suggested to have anti-depressive effects through the removal of glutamate, preventing it from rising to neurotoxic values contributing to neurologic disease, and supplementation has been shown to be associated with earlier improvement in depressive symptoms in bipolar depression.

Higher physical activity individuals were also found to have higher coping abilities through the acceptance of reality when compared to low physical activity participants, and this finding was associated with increased abundance of Megasphaera. Acceptance is a type of adaptive coping strategy that has been shown be negatively correlated with mental illnesses including anxiety and schizophrenia. Furthermore, when comparing the gut microbial communities of post-partum depression individuals and healthy controls, Megasphaera was seen to be enriched in healthy controls. This finding is possibly explained by the metabolizing activity that Megasphaera harbors for propionate, which has shown to improve depressive mood in rodents and is found to be depleted in patients with major depressive disorder (MDD). Based on these results, it is possible that physical activity may promote the development of protective cognitive processes such as coping that decrease the risk of psychiatric illnesses. In comparison, contrary to the findings in our study, Megasphaera was found in higher abundance in both overweight/obese participants and low-activity participants in an Italian study.

Within YFAS food addiction measures, moderate physical activity was associated with the highest food addiction scores while high physical activity participants had the lowest scores, and these findings were associated with altered connectivity within brain regions of the default mode network (DMN). Specifically, the moderate physical activity participants when compared to those with high physical activity had increased connectivity between the angular gyrus and middle temporal gyrus regions, which a previous study demonstrated to be increased in activation when participants passively viewed visual food cues versus while they actively inhibited the urge to eat. Previous studies on the chronic effects of exercise on appetite parameters have been largely conflicting, with some studies reporting an increase in subjective appetite in the fasted state after aerobic exercise, whereas others have reported a reduction or no change. Our findings suggest that the subjective appetite responses to physical activity may be intensity-dependent, with moderate physical activity stimulating appetite and high intensity physical activity reducing appetite. YFAS scores were also associated with Methanobrevibactor, which was shown to be higher in abundance with higher physical activity as consistent with previous findings in both the high versus low and moderate versus low comparisons. Methanobrevibacter smithii allows for increased degradation of organic matter in the colon and has been shown to be depleted in obese individuals in comparison to lean individuals, suggesting that increased physical activity may result in a microbial signature more similar to a lean phenotype and that is better adapted to harvest energy efficiently. Interestingly, in study on individuals with eating disorders, an increased abundance of Methanobrevibacter species were noted in anorexia nervosa (AN) patients in comparison to lean or obese controls. While there is still controversy around whether AN patients experience alterations in appetite and satiety, these findings suggest a potential relationship between Methanobrevibactor and food addiction and may explain the decreased food addiction scores seen in the high physical activity participants who demonstrated increased abundance in Methanobrevibactor.

Several of the significant microbiome genera and fecal metabolites found to be associated with physical activity have previously been studied in the context of psychiatric illnesses. Myoinositol represents a metabolite previously associated with psychiatric disorders and was found to be elevated in the high physical activity group when compared to both moderate and low physical activity. This finding was associated with increased connectivity within the central executive network (CEN), default mode network (DMN), and sensorimotor network (SMN), specifically involving the superior parietal lobule, supramarginal gyrus, inferior part of the precentral sulcus, and postcentral sulcus. In major depressive disorder (MDD) patients, a reduced abundance of myo-inositol is seen compared to healthy controls⁵¹. Myoinositol may be involved in mood regulation by maintaining glial osmoregulatory functions, which is reflected by reduced density of glial cells in MDD patients. Additionally, as consistent with previous studies, Christensenellaceae demonstrated a dose-dependent association with physical activity, with both high physical activity and moderate physical activity indicating increased abundance compared to low physical activity. In previous studies, Christensenellaceae was found to be significantly lower in abundance in a variety of brain-related disorders, including general anxiety disorder, MDD, Parkinson's disease, multiple sclerosis, and autism. These findings suggest that physical activity may promote a microbiome that is protective against developing psychiatric and neurologic disorders through the altering the production of metabolites that exhibit neuroactive properties.

In addition to the neuroprotective associations, Christensenellaceae has one of the most robust and reproducible associations with a lean phenotype and has also been reported to relate to health in various metabolic disorders. Similarly, Prevotella is known to predict increased weight loss in overweight individuals and is linked with dietary-fiber induced improvements in glucose metabolism. As consistent with previous findings, higher Prevotella was associated with more physical activity when comparing high versus low physical activity individuals. Another microbial genera found to be elevated with more physical activity was Akkermansia, which is known to represent a lean-phenotype microbiome and protect against obesity-related metabolic disturbances, possibly by enhancing gut barrier integrity. Additionally, the elevated abundance of Akkermansia associated with high physical activity was found to be correlated to the increased myoinositol in the high physical activity group. The increased abundance of these genera associated with lean-phenotypes further illustrates a widely accepted finding that more physical activity promotes a metabolically healthy microbiome that may prevent further weight gain in already obese individuals.

Physical activity-associated changes were also seen in brain functional connectivity, most notably when comparing the high versus moderate physical activity groups. The superior frontal gyrus (SupFG) and middle frontal gyrus (MFG), which are both frontal lobe regions implicated in general inhibitory control but also appetite control, were increased in connectivity in the high physical activity group compared to moderate physical activity, which is consistent with the clinical findings of lowest food cravings with high physical activity and highest food cravings with moderate physical activity. A possible explanation is that the MFG has also been proposed to act as a circuit-breaker between the ventral and dorsal attention networks, and thus allows for top-down reorientation of attention from endogenous stimuli such as hunger cues to exogenous stimuli in the environment. Similar to our findings, other studies have demonstrated that in comparison to obese individuals, previously obese individuals who successfully maintained weight loss as well as lean individuals have greater activation in the SupFG in response to food cues and during tasks involving response inhibition. SupFG has also been negatively correlated to self-reported impulsivity, with ADHD individuals showing hypoactivity in SupFG and MFG. In addition, more emotional regulation network regions were increased in connectivity in moderate physical activity participants, and were linked to central autonomic versus central executive as seen in the high physical activity group, suggesting more cognitive control over emotional food cravings with more physical activity. There were also overall more CEN regions increased in connectivity with high physical activity linked to the somatosensory (SMN) and default mode networks (DMN), in comparison to the increased connectivity between CEN and occipital regions seen in moderate physical activity individuals. This suggests more cognitive evaluation of sensory stimuli with high physical activity, that may contribute to more restraint and less impulsivity in uncontrolled eating.

Our study had several strengths, including the integration of a comprehensive dataset including brain, gut microbiome, fecal metabolite, and psychosocial variables to determine associations with physical activity level. We also utilized consistent sample processing and OTU clustering and considered major covariates in our analyses. However, the directionality and causality between physical activity and alterations in the BGM system cannot be parsed through this study, but cross-sectional studies such as the one we presented here allow for further understanding the role of physical activity in preventing mental illnesses. Future studies including larger and longitudinal and more evenly distributed sample sizes within each physical activity level group are warranted and would allow for more statistical power in the analyses. In addition, since there have been studies showing the differing effects of aerobic versus resistance exercise on the microbiome and the IPAQ questionnaire used in our cross-sectional study was unable to differentiate between these different forms of exercise, additional studies that distinguished different forms of exercise are also warranted.

To our knowledge, this is the first study to integrate all aspects of the brain-gut microbiome axis to understand mechanistically how physical activity can promote beneficial psychosocial changes within an obese population. With the COVID-19 pandemic creating short and long-term mental health consequences in as much as 30% of the general population and individuals already suffering from a psychiatric disease, it is even more crucial to identify evidence-based methods to promote psychological resilience amidst this ongoing global health crisis. We have identified novel targets within the BGM system that may be explored for the prevention and treatment of various psychiatric conditions, which obese individuals are at higher risk for. This study will inform the design of future longitudinal studies that will elucidate the directionality of these associations.

### Tables

TABLE 1
Participant's clinical and psychosocial characteristics with physical activity-based correlations.

| | | | | | | | | Moderate PA |
|---|---|---|---|---|---|---|---|---|
| | All (N = 92) | | | | High PA (N = 43) | | | (N = 32) |
| | Mean | SD | Range | N | Mean | SD | Range | Mean |
| Age | 32.84 | 10.30 | [18, 54] | 92 | 35.12 | 11.77 | [18, 54] | 29.65 |
| BMI | 33.22 | 4.54 | [25.32, 47.54] | 92 | 33.25 | 4.50 | [25.59, 45.29] | 33.98 |
| Race | | | | | | | | |
| Hispanic | | | | 50 | | | | |
| Asian | | | | 9 | | | | |
| Black | | | | 15 | | | | |
| White | | | | 18 | | | | |
| Diet | | | | | | | | |
| American | | | | 74 | | | | |
| Non- | | | | 18 | | | | |
| American | | | | | | | | |
| | EDUCATION AND SOCIOECONOMIC STATUS | | | | | | | |
| Education | 4.81 | 0.94 | [2, 6] | 89 | 4.60 | 0.94 | [2, 6] | 4.94 |
| | PHYSICAL ACTIVITY (IPAQ) | | | | | | | |
| Total | 3935.06 | 4225.91 | [0, 26099.41] | 92 | 5952.78 | 4801.98 | [609, 26099.41) | 1107.60 |
| Walking | | | | | | | | |
| Total | 2508.86 | 3524.13 | [0, 21840] | 92 | 3870.01 | 4086.79 | [0, 21840] | 457.94 |
| Moderate | | | | | | | | |
| Total | 1938.87 | 4232.45 | [0, 32688] | 92 | 3610.05 | 5685.29 | [0, 32688] | 257.41 |
| Vigorous | | | | | | | | |
| Total | 8382.79 | 8936.20 | [0, 56507.11] | 92 | 13432.84 | 9488.34 | [3834, 56507.11) | 1822.95 |
| Physical | | | | | | | | |
| Activity | | | | | | | | |
| | RESILIENCE | | | | | | | |
| BRS | 22.681 | 4.718 | [9, 30] | 91 | 23.9286 | 4.8760 | [9, 30] | 21.059 |
| Score | | | | | | | | |
| | BRIEF COPE | | | | | | | |
| Self | 4.70 | 1.70 | [2, 8] | 88 | 4.56 | 1.66 | [2, 8] | 4.47 |
| Distraction | | | | | | | | |
| Active | 5.63 | 1.91 | [2, 8] | 88 | 5.78 | 1.98 | [2, 8] | 5.00 |
| Coping | | | | | | | | |
| Denial | 2.38 | 0.93 | [2, 8] | 87 | 2.25 | 0.67 | [2, 5] | 2.41 |
| Substance | 2.20 | 0.79 | [2, 8] | 88 | 2.29 | 1.05 | [2, 8] | 2.12 |
| Use | | | | | | | | |
| Emotional | 5.52 | 2.01 | [2, 8] | 88 | 5.54 | 2.12 | [2, 8] | 5.18 |
| Support | | | | | | | | |
| Intrumental | 5.41 | 2.07 | [2, 8] | 88 | 5.46 | 2.23 | [2, 8] | 4.65 |
| Support | | | | | | | | |
| Behavioral | 2.42 | 0.74 | [2, 5] | 88 | 2.37 | 0.70 | [2, 4] | 2.35 |
| Disengagement | | | | | | | | |
| Venting | 4.20 | 1.68 | [2, 8] | 87 | 4.07 | 1.63 | [2, 8] | 4.38 |
| Positive | 5.28 | 1.94 | [2, 8] | 88 | 5.46 | 1.92 | [2, 8] | 4.5 |
| Reframing | | | | | | | | |
| Planning | 5.84 | 1.73 | [2, 8] | 89 | 6.02 | 1.77 | [2, 8] | 5.18 |
| Humor | 4.30 | 1.77 | [2, 8] | 89 | 4.43 | 1.85 | [2, 8] | 3.76 |
| Acceptance | 5.72 | 1.81 | [2, 8] | 89 | 6.17 | 1.82 | [2, 8] | 5.06 |
| Religion | 4.51 | 2.20 | [2, 8] | 87 | 4.73 | 2.26 | [2, 8] | 3.53 |
| Self | 3.83 | 1.70 | [2, 8] | 89 | 3.95 | 1.86 | [2, 8] | 3.71 |
| Blame | | | | | | | | |
| | STRESS AND ANXIETY | | | | | | | |
| STAI | 47.85 | 10.86 | [33, 86] | 91 | 46.19 | 11.47 | [33, 86] | 49.24 |
| (Anxiety) | | | | | | | | |
| HAD | 5.12 | 3.71 | [0, 14] | 92 | 4.56 | 3.82 | [0, 14] | 5.18 |
| Anxiety | | | | | | | | |
| HAD | 2.46 | 2.60 | [0, 13] | 92 | 1.95 | 2.37 | [0, 13] | 3.06 |
| Depression | | | | | | | | |
| PSS | 13.18 | 6.55 | [1, 33] | 91 | 12.32 | 5.58 | [1, 25] | 14.82 |
| Score | | | | | | | | |
| | MOOD & EMOTION (PANAS) | | | | | | | |
| Positive | 32.12 | 8.53 | [15, 50] | 92 | 34.10 | 8.06 | [16, 49] | 30.38 |
| Affect | | | | | | | | |
| Negative | 12.41 | 3.60 | [10, 29] | 92 | 12.23 | 3.39 | [10, 29] | 13.09 |
| Affect | | | | | | | | |
| | FOOD CRAVINGS (Yale Food Addiction Scale) | | | | | | | |
| Withdrawal | 0.217 | 0.531 | [0, 3] | 92 | 0.16 | 0.43 | [0, 2] | 0.41 |
| Tolerance | 0.205 | 0.529 | [0, 2] | 88 | 0.20 | 0.51 | [0, 2] | 0.44 |
| Continued | 0.239 | 0.429 | [0, 1] | 92 | 0.19 | 0.39 | [0, 1] | 0.47 |
| Use | | | | | | | | |
| Given | 0.165 | 0.601 | [0, 4] | 91 | 0.05 | 0.22 | [0, 1] | 0.59 |
| Up | | | | | | | | |
| Time | 0.304 | 0.588 | [0, 3] | 92 | 0.16 | 0.43 | [0, 2] | 0.82 |
| Spent | | | | | | | | |
| Loss | 0.110 | 0.379 | [0, 2] | 91 | 0.05 | 0.31 | [0, 2] | 0.35 |
| Control | | | | | | | | |
| Unsuccessful | 1.716 | 0.909 | [0, 4] | 88 | 1.64 | 1.01 | [0, 4] | 1.88 |
| Cut | | | | | | | | |
| Down | | | | | | | | |
| ClinSig | 0.120 | 0.415 | [0, 2] | 92 | 0.05 | 0.21 | [0, 1] | 0.24 |
| Impairment | | | | | | | | |
| Symptom | 1.924 | 1.477 | [0, 7] | 92 | 1.60 | 1.12 | [0, 5] | 3.18 |
| Count | | | | | | | | |
| | BODY MASS (Bioimpedance Analysis) | | | | | | | |
| Fat | 35.42 | 8.09 | [3, 51.10] | 90 | 34.10 | 8.78 | [3, 50.10) | 36.84 |
| Mass | | | | | | | | |
| (%) | | | | | | | | |
| Lean | | | | | | | | |
| Body | 64.57 | 8.09 | [48.90, 97] | 90 | 65.88 | 8.77 | (49.90, 97] | 63.17 |
| Mass | | | | | | | | |
| (%) | | | | | | | | |
| Total | 100.00 | 0.00 | [100, 100] | 78 | 100.00 | 0.00 | [100, 100] | 100.00 |
| Weight | | | | | | | | |
| (%) | | | | | | | | |

| | Moderate PA | | Low PA | | | High vs. | Mod vs. | High vs. |
|---|---|---|---|---|---|---|---|---|
| | (N = 32) | | (N = 17) | | | Mod PA | Low PA | Low PA |
| | SD | Range | Mean | SD | Range | p-value | | |
| Age | 8.67 | [19, 54] | 31.47 | 8.12 | [19, 53] | 0.14 | 0.48 | 0.08 |
| BMI | 4.33 | [25.32, 42.07] | 32.77 | 5.19 | [27.27, 47.54] | 0.65 | 0.39 | 0.59 |
| Race | | | | | | 0.76 | 0.87 | 0.57 |
| Hispanic | | | | | | | | |
| Asian | | | | | | | | |
| Black | | | | | | | | |
| White | | | | | | | | |
| Diet | | | | | | 0.60 | 1.00 | 0.90 |
| American | | | | | | | | |
| Non- | | | | | | | | |
| American | | | | | | | | |
| | EDUCATION AND SOCIOECONOMIC STATUS | | | | | | | |
| Education | 0.89 | [3, 6] | 5.03 | 0.97 | [3, 6] | 0.05 | 0.74 | 0.21 |
| | PHYSICAL ACTIVITY (IPAQ) | | | | | | | |
| Total | 2985.03 | [0, 14157] | 2725.83 | 1189.69 | [0, 4223.71) | 1.29E−03 | 0.04 | 1.34E−04 |
| Walking | | | | | | | | |
| Total | 2880.03 | [0, 14669] | 1769.37 | 425.30 | [0, 1260] | 0.02 | 0.07 | 1.16E−03 |
| Moderate | | | | | | | | |
| Total | 1125.42 | [0, 5440] | 586.50 | 644.67 | [0, 2576] | 4.14E−03 | 0.27 | 0.02 |
| Vigorous | | | | | | | | |
| Total | 6364.02 | [772, 34266] | 5081.70 | 1824.55 | [0, 7516.71) | 5.09E−05 | 0.05 | 5.95E−06 |
| Physical | | | | | | | | |
| Activity | | | | | | | | |
| | RESILIENCE | | | | | | | |
| BRS | 4.596 | [13, 30] | 21.906 | 3.913 | [16, 29] | 0.07 | 0.52 | 0.04 |
| Score | | | | | | | | |
| | BRIEF COPE | | | | | | | |
| Self | 1.75 | [2, 8] | 5.03 | 1.70 | [2, 7] | 0.25 | 0.29 | 0.85 |
| Distraction | | | | | | | | |
| Active | 1.91 | [2, 8] | 5.77 | 1.73 | [2, 8] | 0.98 | 0.18 | 0.16 |
| Coping | | | | | | | | |
| Denial | 1.22 | [2, 8] | 2.53 | 0.87 | [2, 5] | 0.22 | 0.72 | 0.45 |
| Substance | 0.43 | [2, 4] | 2.13 | 0.49 | [2, 4] | 0.44 | 0.91 | 0.52 |
| Use | | | | | | | | |
| Emotional | 1.95 | [2, 8] | 5.70 | 1.91 | [2, 8] | 0.74 | 0.38 | 0.55 |
| Support | | | | | | | | |
| Intrumental | 1.94 | [2, 8] | 5.77 | 1.80 | [2, 8] | 0.55 | 0.06 | 0.19 |
| Support | | | | | | | | |
| Behavioral | 0.86 | [2, 5] | 2.53 | 0.61 | [2, 4] | 0.37 | 0.45 | 0.95 |
| Disengagement | | | | | | | | |
| Venting | 1.76 | [2, 8] | 4.27 | 1.75 | [2, 7] | 0.63 | 0.84 | 0.54 |
| Positive | 2.03 | [2, 8] | 5.47 | 1.70 | [2, 7] | 0.09 | 0.1 | 0.09 |
| Reframing | | | | | | | | |
| Planning | 1.59 | [2, 8] | 5.97 | 1.81 | [2, 8] | 0.89 | 0.13 | 0.10 |
| Humor | 1.77 | [2, 8] | 4.43 | 1.56 | [2, 6] | 0.99 | 0.20 | 0.20 |
| Acceptance | 1.74 | [2, 8] | 5.47 | 1.71 | [2, 8] | 0.11 | 0.44 | 0.04 |
| Religion | 2.23 | [2, 8] | 4.68 | 1.85 | [2, 8] | 0.92 | 0.09 | 0.07 |
| Self | 1.57 | [2, 8] | 3.73 | 1.57 | [2, 8] | 0.60 | 0.95 | 0.63 |
| Blame | | | | | | | | |
| | STRESS AND ANXIETY | | | | | | | |
| STAI | 10.75 | [33, 78] | 49.28 | 9.43 | [33, 65] | 0.24 | 0.99 | 0.34 |
| (Anxiety) | | | | | | | | |
| HAD | 3.61 | [0, 14] | 5.84 | 3.56 | [1, 13] | 0.14 | 0.54 | 0.57 |
| Anxiety | | | | | | | | |
| HAD | 2.56 | [0, 10] | 2.81 | 3.11 | [0, 10] | 0.14 | 0.77 | 0.14 |
| Depression | | | | | | | | |
| PSS | 7.93 | [1, 33] | 13.44 | 5.92 | [2, 25] | 0.48 | 0.53 | 0.13 |
| Score | | | | | | | | |
| | MOOD & EMOTION (PANAS) | | | | | | | |
| Positive | 8.77 | [15, 50] | 30.41 | 8.62 | [16, 49] | 0.06 | 0.99 | 0.12 |
| Affect | | | | | | | | |
| Negative | 4.48 | [10, 27] | 11.59 | 1.73 | [10, 16] | 0.35 | 0.19 | 0.46 |
| Affect | | | | | | | | |
| | FOOD CRAVINGS (Yale Food Addiction Scale) | | | | | | | |
| Withdrawal | 0.40 | [0, 1] | 0.19 | 0.87 | [0, 3] | 0.80 | 0.22 | 0.14 |
| Tolerance | 0.40 | [0, 2] | 0.10 | 0.73 | [0, 2] | 0.38 | 0.04 | 0.16 |
| Continued | 0.40 | [0, 1] | 0.19 | 0.51 | [0, 1] | 0.99 | 0.04 | 0.02 |
| Use | | | | | | | | |
| Given | 0.39 | [0, 2] | 0.09 | 1.18 | [0, 4] | 0.52 | 0.03 | 0.01 |
| Up | | | | | | | | |
| Time | 0.42 | [0, 1] | 0.22 | 0.88 | [0, 3] | 0.58 | 2.07E−03 | 0.00 |
| Spent | | | | | | | | |
| Loss | 0.25 | [0, 1] | 0.06 | 0.61 | [0, 2] | 0.82 | 0.02 | 0.01 |
| Control | | | | | | | | |
| Unsuccessful | 0.80 | [1, 4] | 1.72 | 0.86 | [1, 4] | 0.72 | 0.53 | 0.39 |
| Cut | | | | | | | | |
| Down | | | | | | | | |
| ClinSig | 0.51 | [0, 2] | 0.16 | 0.56 | [0, 2] | 0.21 | 0.62 | 0.06 |
| Impairment | | | | | | | | |
| Symptom | 1.28 | [0, 6] | 1.69 | 1.98 | [1, 7] | 0.77 | 2.50E−03 | 2.52E−04 |
| Count | | | | | | | | |
| | BODY MASS (Bioimpedance Analysis) | | | | | | | |
| Fat | 6.73 | (20.29, 51.10) | 36.43 | 8.56 | (14.10, 47.60) | 0.22 | 0.86 | 0.28 |
| Mass | | | | | | | | |
| (%) | | | | | | | | |
| Lean | | | | | | | | |
| Body | 6.73 | (48.90, 79.70) | 63.57 | 8.56 | (52.40, 85.91) | 0.23 | 0.86 | 0.28 |
| Mass | | | | | | | | |
| (%) | | | | | | | | |
| Total | 0.00 | [100, 100] | 100.00 | 0.00 | [100, 100] | 0.42 | 0.45 | 0.54 |
| Weight | | | | | | | | |
| (%) | | | | | | | | |

Means and standard deviations are reported for normally distributed data.
p-significant < 0.05.
International Physical Activity Questionnaire (IPAQ), Brief Resilience Scale (BRS), Perceived Stress Scale (PSS), Hospital Anxiety and Depression Scale (HADS), Yale Food Addiction Scale (YFAS), Positive and Negative Affect Schedule (PANAS), Brief-COPE (BCope), State-Trait Anxiety Inventory (STAI)

Means and standard deviations are reported for normally distributed data. p-significant <0.05

### International Physical Activity Questionnaire (IPAQ), Brief Resilience Scale (BRS), Perceived Stress Scale

### (PSS), Hospital Anxiety and Depression Scale (HADS), Yale Food Addiction Scale (YFAS)

TABLE 2
Summary of Functional Brain Connectivity Differences Based on Physical Activity Levels.

| | | High v.s Moderate | | | | |
|---|---|---|---|---|---|---|
| Variable A | Variable B | MD | F | p | q | Int. |

Brain Component 1

| | | | | | | | | |
|---|---|---|---|---|---|---|---|---|
| SAL | R_MPoCgG_S | SMN | R_SbCG_S | 0.17 | 14.80 | 2.63E−04 | 0.002 | High ↑ |
| CAN | L_OrG | DMN | L_SupTS | 0.16 | 13.83 | 4.04E−04 | 0.002 | High ↑ |
| CEN | R_MFG | CEN | R_IntPS_TrPS | −0.18 | 6.45 | 0.013 | 0.019 | Mod ↑ |
| CEN | R_MFG | DMN | R_MTG | −0.17 | 7.12 | 0.010 | 0.015 | Mod ↑ |
| CEN | R_SupPL | OCC | R_SupOcG | −0.15 | 6.39 | 0.014 | 0.019 | Mod ↑ |
| CEN | R_SupPL | OCC | R_SupOcG | −0.16 | 6.32 | 0.014 | 0.019 | Mod ↑ |
| CEN | R_SupPL | CEN | R_SupPL | 0.16 | 3.20 | 0.078 | 0.078 | High ↑ |
| CEN | R_SupPL | DMN | R_SuMarG | 0.22 | 13.88 | 3.95E−04 | 0.002 | High ↑ |
| CEN | L_SbPS | ERN | L_InfFS | 0.18 | 13.41 | 4.86E−04 | 0.002 | High ↑ |
| CEN | R_POcS | OCC | R_SupOcG | 0.19 | 9.41 | 0.003 | 0.007 | High ↑ |
| CEN | L_MFG | SMN | L_SupFG | 0.16 | 17.14 | 9.66E−05 | 0.001 | High ↑ |
| CEN | L_MFG | SMN | L_SupFG | 0.18 | 15.53 | 1.92E−04 | 0.002 | High ↑ |
| CEN | L_MFG | DMN | L_PrCun | 0.17 | 15.69 | 1.79E−04 | 0.002 | High ↑ |
| CEN | R_MFG | SMN | R_PRCG | 0.16 | 14.27 | 3.32E−04 | 0.002 | High ↑ |
| CEN | R_MFG | DMN | R_CgSMarp | 0.17 | 17.84 | 7.21E−05 | 0.001 | High ↑ |
| CEN | R_MFG | SMN | R_PosCG | 0.16 | 27.37 | 1.72E−06 | 6.01E−05 | High ↑ |
| ERN | R_ACgG_S | CAN | L_SbOrS | −0.14 | 4.86 | 0.031 | 0.034 | Mod ↑ |
| ERN | L_ACgG_S | CAN | L_SbOrS | −0.19 | 6.70 | 0.012 | 0.017 | Mod ↑ |
| ERN | L_InfFS | DMN | L_AngG | 0.15 | 5.43 | 0.023 | 0.026 | High ↑ |
| SMN | R_SupFS | CEN | R_MFG | 0.15 | 10.30 | 0.002 | 0.005 | High ↑ |
| SMN | L_SupFS | OCC | L_AOcS | 0.17 | 14.57 | 2.91E−04 | 0.002 | High ↑ |
| SMN | L_InfPrCS | SMN | L_PosCS | 0.19 | 11.83 | 0.001 | 0.003 | High ↑ |
| SMN | R_SupFG | CEN | R_MFG | 0.16 | 5.65 | 0.020 | 0.025 | High ↑ |
| SMN | R_SupFG | CAN | R_OrG | 0.18 | 5.59 | 0.021 | 0.025 | High ↑ |
| SMN | R_SupFG | CAN | R_OrG | 0.18 | 5.57 | 0.021 | 0.025 | High ↑ |
| SMN | R_SupFG | SMN | R_PosCG | 0.17 | 15.01 | 2.41E−04 | 0.002 | High ↑ |
| SMN | R_SupFG | CEN | R_MFG | 0.16 | 6.21 | 0.015 | 0.020 | High ↑ |
| DMN | L_PrCun | OCC | L_LinG | 0.17 | 10.76 | 0.002 | 0.005 | High ↑ |
| DMN | L_PrCun | OCC | L_CcS | 0.17 | 10.72 | 0.002 | 0.005 | High ↑ |
| DMN | L_PrCun | OCC | L_LinG | 0.17 | 6.97 | 0.010 | 0.015 | High ↑ |
| DMN | R_AngG | OCC | L_SupOcG | 0.15 | 5.37 | 0.023 | 0.026 | High ↑ |
| DMN | R_PrCun | OCC | R_SupOcG | 0.18 | 8.41 | 0.005 | 0.009 | High ↑ |
| DMN | R_PrCun | OCC | R_SupOcG | 0.21 | 15.67 | 1.81E−04 | 0.002 | High ↑ |
| DMN | R_PrCun | OCC | R_SupOcG | 0.16 | 8.75 | 0.004 | 0.008 | High ↑ |
| DMN | L_SupTS | OCC | L_AOcS | 0.18 | 10.12 | 0.002 | 0.005 | High ↑ |
| DMN | R_MTG | OCC | L_SupOcS_TrOcS | 0.15 | 10.25 | 0.002 | 0.005 | High ↑ |
| DMN | L_SupTS | OCC | L_SupOcS_TrOcS | 0.17 | 13.04 | 0.001 | 0.002 | High ↑ |
| DMN | L_SupTGLp | SMN | L_SupFG | 0.16 | 7.24 | 0.009 | 0.014 | High ↑ |
| DMN | R_SuMarG | SMN | L_SupFG | 0.18 | 12.83 | 0.001 | 0.002 | High ↑ |
| DMN | R_SupTS | SMN | L_SupFG | 0.17 | 10.29 | 0.002 | 0.005 | High ↑ |
| DMN | R_SupTS | DMN | R_MTG | 0.19 | 17.30 | 9.04E−05 | 0.001 | High ↑ |
| DMN | R_SupTS | DMN | R_SupTS | −0.16 | 5.58 | 0.021 | 0.025 | Mod ↑ |
| DMN | R_SupTS | DMN | R_SupTS | −0.17 | 10.06 | 0.002 | 0.005 | Mod ↑ |
| DMN | R_SuMarG | DMN | R_MTG | 0.21 | 7.33 | 0.009 | 0.014 | High ↑ |
| DMN | L_SupTS | DMN | L_TPl | 0.16 | 3.83 | 0.054 | 0.055 | High ↑ |
| DMN | R_SupTGLp | DMN | R_MTG | 0.18 | 13.61 | 4.45E−04 | 0.002 | High ↑ |
| DMN | R_SupTS | DMN | R_AngG | 0.17 | 7.93 | 0.006 | 0.011 | High ↑ |
| DMN | R_MTG | DMN | L_TP1 | 0.17 | 8.52 | 0.005 | 0.009 | High ↑ |
| DMN | R_MTG | DMN | L_TP1 | 0.19 | 5.89 | 0.018 | 0.023 | High ↑ |
| DMN | R_MTG | DMN | R_SuMarG | 0.16 | 3.90 | 0.052 | 0.055 | High ↑ |
| DMN | R_MTG | DMN | L_SupTGLp | 0.17 | 9.08 | 0.004 | 0.007 | High ↑ |
| DMN | R_MTG | DMN | L_AngG | −0.13 | 4.80 | 0.032 | 0.035 | Mod ↑ |
| DMN | R_AngG | DMN | R_MTG | −0.19 | 10.78 | 0.002 | 0.005 | Mod ↑ |
| DMN | L_PosDCgG | DMN | L_InfTS | −0.17 | 8.15 | 0.006 | 0.010 | Mod ↑ |
| DMN | R_MTG | CEN | R_IntPS_TrPS | −0.19 | 9.94 | 0.002 | 0.005 | Mod ↑ |
| DMN | R_SupTS | ERN | L_PaHipG | −0.17 | 27.69 | 1.52E−06 | 6.01E−05 | Mod ↑ |
| OCC | R_Cun | DMN | L_PrCun | 0.17 | 7.58 | 0.008 | 0.013 | High ↑ |
| OCC | R_LinG | DMN | L_PrCun | 0.15 | 8.53 | 0.005 | 0.009 | High ↑ |
| OCC | R_SupOcG | CEN | L_IntPS_TrPS | 0.18 | 9.62 | 0.003 | 0.006 | High ↑ |
| OCC | R_SupOcG | CEN | R_POcS | 0.16 | 3.87 | 0.053 | 0.055 | High ↑ |
| OCC | R_SupOcG | DMN | L_PrCun | 0.18 | 7.94 | 0.006 | 0.011 | High ↑ |
| OCC | R_SupOcG | DMN | L_MTG | 0.17 | 11.93 | 0.001 | 0.003 | High ↑ |
| OCC | R_SupOcG | CEN | R_POcS | 0.18 | 6.42 | 0.014 | 0.019 | High ↑ |
| OCC | R_SupOcG | OCC | R_Cun | 0.18 | 4.77 | 0.032 | 0.035 | High ↑ |
| OCC | R_SupOcG | CEN | L_POcS | 0.17 | 12.93 | 0.001 | 0.002 | High ↑ |
| OCC | R_SupOcG | OCC | L_Cun | 0.17 | 4.67 | 0.034 | 0.036 | High ↑ |
| OCC | R_SupOcG | DMN | L_SupTS | 0.15 | 12.23 | 0.001 | 0.003 | High ↑ |
| OCC | L_Cun | OCC | L_LinG | 0.16 | 5.73 | 0.019 | 0.024 | High ↑ |
| OCC | R_MOcG | OCC | R_SupOcS_TrOcS | −0.16 | 6.98 | 0.010 | 0.015 | Mod ↑ |
| OCC | R_InfOcG_S | OCC | L_SupOcG | −0.19 | 6.16 | 0.016 | 0.020 | Mod ↑ |

Brain Component 2

| | | | | | | | | |
|---|---|---|---|---|---|---|---|---|
| CEN | L_SupPL | OCC | L_Cun | −0.08 | 4.73 | 0.033 | 0.099 | Mod ↑ |
| DMN | R_MTG | DMN | R_AngG | −0.03 | 0.00 | 0.967 | 0.967 | Mod ↑ |
| DMN | L_SuMarG | CEN | L_SupPL | 0.08 | 3.17 | 0.079 | 0.119 | High ↑ |

| | Moderate v.s Low | | | | | High v.s. Low | | | | |
|---|---|---|---|---|---|---|---|---|---|---|
| Variable A | MD | F | p | q | Int. | MD | F | p | q | Int. |

Brain Component 1

| | | | | | | | | | | |
|---|---|---|---|---|---|---|---|---|---|---|
| SAL | −0.07 | 1.58 | 0.21 | 0.60 | Low ↑ | 0.10 | 1.92 | 0.17 | 0.26 | High ↑ |
| CAN | −0.09 | 2.82 | 0.10 | 0.54 | Low ↑ | 0.08 | 1.81 | 0.18 | 0.27 | High ↑ |
| CEN | 0.01 | 0.01 | 0.93 | 0.99 | Mod ↑ | −0.17 | 4.02 | 0.05 | 0.14 | Low ↑ |
| CEN | 0.01 | 0.09 | 0.77 | 0.96 | Mod ↑ | −0.17 | 4.03 | 0.05 | 0.14 | Low ↑ |
| CEN | 2.27E−03 | 0.03 | 0.86 | 0.97 | Mod ↑ | −0.15 | 4.32 | 0.04 | 0.14 | Low ↑ |
| CEN | 0.03 | 0.14 | 0.71 | 0.94 | Mod ↑ | −0.12 | 2.80 | 0.10 | 0.19 | Low ↑ |
| CEN | −0.04 | 0.04 | 0.84 | 0.96 | Low ↑ | 0.12 | 1.60 | 0.21 | 0.28 | High ↑ |
| CEN | −0.06 | 0.70 | 0.41 | 0.86 | Low ↑ | 0.16 | 5.86 | 0.02 | 0.10 | High ↑ |
| CEN | −0.05 | 0.79 | 0.38 | 0.83 | Low ↑ | 0.13 | 5.29 | 0.03 | 0.10 | High ↑ |
| CEN | −0.05 | 0.60 | 0.44 | 0.87 | Low ↑ | 0.14 | 2.62 | 0.11 | 0.19 | High ↑ |
| CEN | −0.05 | 0.88 | 0.35 | 0.83 | Low ↑ | 0.11 | 3.06 | 0.09 | 0.18 | High ↑ |
| CEN | −0.13 | 5.73 | 0.02 | 0.44 | Low ↑ | 0.05 | 0.50 | 0.48 | 0.54 | High ↑ |
| CEN | −0.10 | 4.31 | 0.04 | 0.44 | Low ↑ | 0.06 | 0.54 | 0.47 | 0.53 | High ↑ |
| CEN | −0.03 | 0.36 | 0.55 | 0.94 | Low ↑ | 0.12 | 5.03 | 0.03 | 0.10 | High ↑ |
| CEN | −0.11 | 5.06 | 0.03 | 0.44 | Low ↑ | 0.06 | 0.97 | 0.33 | 0.41 | High ↑ |
| CEN | −0.09 | 3.42 | 0.07 | 0.50 | Low ↑ | 0.08 | 1.67 | 0.20 | 0.28 | High ↑ |
| ERN | −0.06 | 0.82 | 0.37 | 0.83 | Low ↑ | −0.20 | 6.51 | 0.01 | 0.10 | Low ↑ |
| ERN | 0.16 | 4.79 | 0.03 | 0.44 | Mod ↑ | −0.03 | 0.03 | 0.85 | 0.85 | Low ↑ |
| ERN | −2.47E−03 | 0.14 | 0.71 | 0.94 | Low ↑ | 0.15 | 4.76 | 0.03 | 0.11 | High ↑ |
| SMN | −1.18E−03 | 0.06 | 0.81 | 0.96 | Low ↑ | 0.15 | 5.71 | 0.02 | 0.10 | High ↑ |
| SMN | −0.06 | 1.28 | 0.26 | 0.67 | Low ↑ | 0.12 | 3.77 | 0.06 | 0.15 | High ↑ |
| SMN | −0.12 | 7.13 | 0.01 | 0.37 | Low ↑ | 0.07 | 0.90 | 0.35 | 0.41 | High ↑ |
| SMN | 0.06 | 0.42 | 0.52 | 0.94 | Mod ↑ | 0.22 | 6.92 | 0.01 | 0.10 | High ↑ |
| SMN | −0.03 | 0.16 | 0.69 | 0.94 | Low ↑ | 0.16 | 2.78 | 0.10 | 0.19 | High ↑ |
| SMN | −0.10 | 1.95 | 0.17 | 0.56 | Low ↑ | 0.08 | 0.29 | 0.59 | 0.62 | High ↑ |
| SMN | −0.06 | 0.58 | 0.45 | 0.87 | Low ↑ | 0.11 | 5.13 | 0.03 | 0.10 | High ↑ |
| SMN | −0.03 | 0.28 | 0.60 | 0.94 | Low ↑ | 0.13 | 2.11 | 0.15 | 0.25 | High ↑ |
| DMN | −0.04 | 0.23 | 0.64 | 0.94 | Low ↑ | 0.13 | 3.33 | 0.07 | 0.17 | High ↑ |
| DMN | −0.03 | 0.14 | 0.71 | 0.94 | Low ↑ | 0.13 | 3.59 | 0.06 | 0.16 | High ↑ |
| DMN | −0.02 | 0.06 | 0.81 | 0.96 | Low ↑ | 0.15 | 3.02 | 0.09 | 0.18 | High ↑ |
| DMN | 0.02 | 0.07 | 0.79 | 0.96 | Mod ↑ | 0.17 | 4.24 | 0.04 | 0.14 | High ↑ |
| DMN | −0.12 | 2.07 | 0.16 | 0.56 | Low ↑ | 0.06 | 0.37 | 0.54 | 0.58 | High ↑ |
| DMN | −0.02 | 0.01 | 0.92 | 0.99 | Low ↑ | 0.19 | 6.94 | 0.01 | 0.10 | High ↑ |
| DMN | −0.01 | 0.04 | 0.83 | 0.96 | Low ↑ | 0.14 | 5.16 | 0.03 | 0.10 | High ↑ |
| DMN | −0.04 | 0.20 | 0.66 | 0.94 | Low ↑ | 0.14 | 6.62 | 0.01 | 0.10 | High ↑ |
| DMN | −0.01 | 0.01 | 0.94 | 0.99 | Low ↑ | 0.15 | 6.95 | 0.01 | 0.10 | High ↑ |
| DMN | −0.02 | 0.18 | 0.67 | 0.94 | Low ↑ | 0.15 | 6.55 | 0.01 | 0.10 | High ↑ |
| DMN | −0.05 | 1.27 | 0.27 | 0.67 | Low ↑ | 0.11 | 2.37 | 0.13 | 0.22 | High ↑ |
| DMN | −0.07 | 1.70 | 0.20 | 0.58 | Low ↑ | 0.11 | 1.91 | 0.17 | 0.26 | High ↑ |
| DMN | −0.09 | 4.61 | 0.04 | 0.44 | Low ↑ | 0.08 | 0.99 | 0.33 | 0.41 | High ↑ |
| DMN | −0.09 | 3.08 | 0.09 | 0.50 | Low ↑ | 0.11 | 3.38 | 0.07 | 0.17 | High ↑ |
| DMN | 0.04 | 0.31 | 0.58 | 0.94 | Mod ↑ | −0.12 | 3.13 | 0.08 | 0.18 | Low ↑ |
| DMN | 0.09 | 1.81 | 0.19 | 0.56 | Mod ↑ | −0.08 | 1.64 | 0.21 | 0.28 | Low ↑ |
| DMN | 0.01 | 1.73E−04 | 0.99 | 1.00 | Mod ↑ | 0.22 | 6.20 | 0.02 | 0.10 | High ↑ |
| DMN | −0.04 | 0.13 | 0.72 | 0.94 | Low ↑ | 0.13 | 1.96 | 0.17 | 0.26 | High ↑ |
| DMN | −0.08 | 1.88 | 0.18 | 0.56 | Low ↑ | 0.10 | 1.20 | 0.28 | 0.37 | High ↑ |
| DMN | −0.10 | 3.13 | 0.08 | 0.50 | Low ↑ | 0.07 | 0.57 | 0.46 | 0.53 | High ↑ |
| DMN | −0.05 | 0.85 | 0.36 | 0.83 | Low ↑ | 0.13 | 2.72 | 0.11 | 0.19 | High ↑ |
| DMN | −0.05 | 0.63 | 0.43 | 0.87 | Low ↑ | 0.14 | 2.10 | 0.15 | 0.25 | High ↑ |
| DMN | −0.03 | 0.12 | 0.73 | 0.94 | Low ↑ | 0.13 | 1.94 | 0.17 | 0.26 | High ↑ |
| DMN | −0.09 | 1.39 | 0.25 | 0.66 | Low ↑ | 0.08 | 0.94 | 0.34 | 0.41 | High ↑ |
| DMN | −0.10 | 2.07 | 0.16 | 0.56 | Low ↑ | −0.23 | 12.48 | 8.53E−04 | 0.06 | Low ↑ |
| DMN | 0.11 | 1.96 | 0.17 | 0.56 | Mod ↑ | −0.07 | 0.91 | 0.34 | 0.41 | Low ↑ |
| DMN | 0.00 | 0.01 | 0.92 | 0.99 | Mod ↑ | −0.17 | 7.23 | 0.01 | 0.10 | Low ↑ |
| DMN | 0.05 | 0.20 | 0.66 | 0.94 | Mod ↑ | −0.14 | 3.77 | 0.06 | 0.15 | Low ↑ |
| DMN | 0.11 | 7.23 | 0.01 | 0.37 | Mod ↑ | −0.06 | 3.14 | 0.08 | 0.18 | Low ↑ |
| OCC | −0.04 | 0.22 | 0.64 | 0.94 | Low ↑ | 0.13 | 2.65 | 0.11 | 0.19 | High ↑ |
| OCC | −9.30E−03 | 3.03E−03 | 0.96 | 1.00 | Low ↑ | 0.15 | 5.16 | 0.03 | 0.10 | High ↑ |
| OCC | −0.10 | 3.17 | 0.08 | 0.50 | Low ↑ | 0.09 | 0.92 | 0.34 | 0.41 | High ↑ |
| OCC | 0.05 | 0.37 | 0.55 | 0.94 | Mod ↑ | 0.22 | 5.72 | 0.02 | 0.10 | High ↑ |
| OCC | −0.06 | 0.54 | 0.46 | 0.88 | Low ↑ | 0.12 | 1.61 | 0.21 | 0.28 | High ↑ |
| OCC | −0.01 | 1.53E−03 | 0.97 | 1.00 | Low ↑ | 0.16 | 5.60 | 0.02 | 0.10 | High ↑ |
| OCC | −0.12 | 2.07 | 0.16 | 0.56 | Low ↑ | 0.06 | 0.47 | 0.50 | 0.54 | High ↑ |
| OCC | −0.13 | 2.15 | 0.15 | 0.56 | Low ↑ | 0.05 | 0.13 | 0.72 | 0.73 | High ↑ |
| OCC | −0.10 | 3.76 | 0.06 | 0.50 | Low ↑ | 0.07 | 0.52 | 0.48 | 0.54 | High ↑ |
| OCC | −0.13 | 1.87 | 0.18 | 0.56 | Low ↑ | 0.05 | 0.18 | 0.68 | 0.70 | High ↑ |
| OCC | −0.01 | 0.04 | 0.84 | 0.96 | Low ↑ | 0.14 | 9.06 | 3.96E−03 | 0.10 | High ↑ |
| OCC | −0.02 | 0.15 | 0.70 | 0.94 | Low ↑ | 0.14 | 2.86 | 0.10 | 0.19 | High ↑ |
| OCC | 2.68E−03 | 7.53E−07 | 1.00 | 1.00 | Mod ↑ | −0.16 | 5.52 | 0.02 | 0.10 | Low ↑ |
| OCC | 0.10 | 2.05 | 0.16 | 0.56 | Mod ↑ | −0.09 | 0.38 | 0.54 | 0.58 | Low ↑ |

Brain Component 2

| | | | | | | | | | | |
|---|---|---|---|---|---|---|---|---|---|---|
| CEN | 0.21 | 14.12 | 0.001 | 0.002 | Mod ↑ | 0.128 | 2.288 | 0.136 | 0.136 | High ↑ |
| DMN | −0.17 | 5.47 | 0.024 | 0.024 | Low ↑ | −0.198 | 5.952 | 0.018 | 0.054 | Low ↑ |
| DMN | −0.22 | 10.27 | 0.003 | 0.004 | Low ↑ | −0.141 | 3.198 | 0.079 | 0.119 | Low ↑ |

Comparisons of brain connectivity between each pair of brain regions are made between high vs. moderate, moderate vs. low, and high vs. low physical activity.
MD = Mean Difference
Int = Interpretation
F = F value
p = p-value significant < 0.05
q = q-values derived from FDR correction, q-value significant < 0.05
Networks. SMN: sensorimotor, DMN: default mode, SAL: salience, CEN: central executive, CAN: central autonomic, ERN: emotion regulation, OCC: occipital.
Brain regions: Cathy will help w/writing out all brain abbreviations.
MD = Mean Difference
Int = Interpretation
F = F value
p = p-value significant < 0.05
q = q-values derived from FDR correction, q-value significant < 0.05

Networks: SMN=sensorimotor, DMN=default mode, SAL=salience, CEN=central executive, CAN=central autonomic, ERN=emotion regulation, OCC=occipital.
Brain regions: R_MPosCgG_S=Right Middle-posterior part of the cingulate gyrus and sulcus (pMCC), L_OrG=Left Orbital gyri, R_SupPL=Right Superior parietal lobule (lateral part of P1), L_SbPS=Left Subparietal sulcus, R_POcS=Right Parieto-occipital sulcus (or fissure), L_MFG=Left Middle frontal gyrus (F2), R_ACgG_S=Right Anterior part of the cingulate gyrus and sulcus (ACC), L_ACgG_S=Left Anterior part of the cingulate gyrus and sulcus (ACC), L_InfFS=Left Inferior frontal sulcus, R_SupFS=Right Superior frontal sulcus, L_SupFS=Left Superior frontal sulcus, L_InfPrCS=Left Inferior part of theprecentral sulcus, R_SupFG=Right Superior frontal gyrus (F1), L_PrCun=Left Precuneus (medial part of P1), R_PrCun=Right Precuneus (medial part of P1), L_SupTS=Left Superior temporal sulcus (parallel sulcus), R_SupTS=Right Superior temporal sulcus (parallel sulcus), R_SupTGLp=Right Lateral aspect of the superior temporal gyrus, R_AngG=Right Angular gyrus, L_PosDCgG=Left Posterior-dorsal part of the cingulate gyrus (dPCC), R_Cun=Right Cuneus (O6), R_LinG=Right Lingual gyrus, ligual part of the medial occipito-temporal gyrus, (O5), R_MOcG=Right Middle occipital gyrus (O2, lateral occipital gyrus), R_InfOcG_S=Right Inferior occipital gyrus (O3) and sulcus, L_SuMarG=Left Supramarginal gyrus, R_SbCG_S=Right Subcentral gyrus (central operculum) and sulci, R_SupOcG=Right Superior occipital gyrus (O1), R_PRCG=Right Precentral gyrus, R_CgSMarp=Right Marginal branch (or part) of the cingulate sulcus, L_SbOrS=Left Suborbital sulcus (sulcus rostrales, supraorbital sulcus), R_MFG=Right Middle frontal gyrus (F2), L_AOcS=Left Anterior occipital sulcus and preoccipital notch (temporo-occipital incisure), L_PosCS=Left Postcentral sulcus, R_OrG=Right Orbital gyri, R_PosCG=Right Postcentral gyrus, L_CcS=Left Calcarine sulcus, L_LinG=Left Lingual gyrus, ligual part of the medial occipito-temporal gyrus, (O5), L_SupOcG=Left Superior occipital gyrus (O1), L_SupOcS_TrOcS=Left Superior occipital sulcus and transverse occipital sulcus, L_SupFG=Left Superior frontal gyms (F1), R_MTG=Right Middle temporal gyrus (T2), L_TP1=Left Planum temporale or temporal plane of the superior temporal gyrus, R_SuMarG=Right Supramarginal gyrus, L_SupTGLp=Left Lateral aspect of the superior temporal gyrus, L_AngG=Left Angular gyrus, L_InfTS=Left Inferior temporal sulcus, R_IntPS_TrPS=Right Intraparietal sulcus (interparietal sulcus) and transverse parietal sulci, L_PaHipG=Left Parahippocampal gyrus, parahippocampal part of the medial occipito-temporal gyrus, (T5), L_IntPS_TrPS=Left Intraparietal sulcus (interparietal sulcus) and transverse parietal sulci, L_MTG=Left Middle temporal gyrus (T2), L_POcS=Left Parieto-occipital sulcus (or fissure), L_Cun=Left Cuneus (06), R_SupOcS_TrOcS=Right Superior occipital sulcus and transverse occipital sulcus, L_SupPL=Left Superior parietal lobule (lateral part of P1)

TABLE 3
Differences in Microbiome Relative Abundance Based on Physical Activity Levels.

| | High vs. Low | | | | Mod vs. Low | | | |
|---|---|---|---|---|---|---|---|---|
| | log2Fold | | | | log2Fold | | | |
| Genus | Change | p-adj | Q | Interpretation | Change | p-adj | Q | Interpretation |
| Methanobrevibacter | 24.56 | 1.78E−19 | 1.6E−19 | High > Low | 23.67 | 2.31E−22 | 2.17E−22 | Mod > Low |
| Megasphaera | 24.03 | 2.89E−12 | 2.7E−12 | High > Low | 24.52 | 1.28E−11 | 1.20E−11 | Mod > Low |
| Ruminiclostridium 6 | 8.11 | 1.97E−04 | 1.8E−04 | High > Low | 6.18 | 6.46E−03 | 6.07E−03 | Mod > Low |
| Christensenellaceae | 23.69 | 4.35E−12 | 4.0E−12 | High > Low | 20.63 | 1.28E−11 | 1.20E−11 | Mod > Low |
| Prevotella 9 | 8.47 | 9.02E−09 | 8.3E−09 | High > Low | | | | |
| Akkermansia | 4.06 | 0.04 | 0.04 | High > Low | | | | |
| Anaerostipes | −1.05 | 0.04 | 0.04 | Low < High | | | | |
| Paraprevotella | | | | | 7.66 | 0.02 | 0.02 | Mod > Low |
| Ruminococcaceae | | | | | 7.48 | 0.02 | 0.02 | Mod > Low |
| UCG-014 | | | | | | | | |

The microbiome genera with q-significant (q < 0.05) Log2fold changes indicating differences in relative abundance when comparing between high vs. low or moderate vs. low physical activity levels are displayed.
No significant differences were observed in the high vs. moderate comparisons.
The interpretation column indicates which physical activity level group had higher relative abundances for that specific genera.
p = p-value significant <0.05
q = q-values derived from FDR correction, q-value significant <0.05

TABLE 4
Fecal metabolites Associated with Physical Activity Levels.

| | | | | | | High vs. Moderate | | | | | |
|---|---|---|---|---|---|---|---|---|---|---|---|
| | Super | Sub | High vs. Low | | | | | Inter- | Moderate vs. Low | | |
| Metabolite | Pathway | Pathway | p | q | Int. | p | q | pretation | p | q | Int. |
| 5alpha-androstan- | Lipid | And. | 0.02 | 0.03 | High > | 2.38E−03 | 0.02 | High > | | | |
| 3beta, 17alpha-diol | | Steroids | | | Low | | | Mod | | | |
| monosulfate | | | | | | | | | | | |
| myo-inositol | Lipid | Inositol | 0.01 | 0.03 | High > | 0.04 | 0.05 | High > | | | |
| | | Met. | | | Low | | | Mod | | | |
| 5alpha-pregnan- | Lipid | Progestin | 0.02 | 0.03 | Low > | | | | | | |
| 3beta, 20alpha-diol | | Steroids | | | High | | | | | | |
| disulfate | | | | | | | | | | | |
| 5alpha-pregnan- | Lipid | Progestin | 0.01 | 0.03 | Low > | | | | | | |
| 3beta, 20alpha-diol | | Steroids | | | High | | | | | | |
| monosulfate | | | | | | | | | | | |
| chenodeoxycholic | Lipid | Primary | 0.04 | 0.06 | Low > | 0.04 | 0.04 | Mod > | | | |
| acid sulfate | | BA Met. | | | High | | | High | | | |
| cholate sulfate | Lipid | Primary | 0.02 | 0.06 | Low > | 0.02 | 0.04 | Mod > | | | |
| | | BA Met. | | | High | | | High | | | |
| taurochenodeoxycholate | Lipid | Primary | | | | | | | 0.03 | 0.03 | Mod > Low |
| | | BA Met. | | | | | | | | | |
| taurolithocholate | Lipid | Secondary | | | | | | | 0.02 | 0.03 | Low > Mod |
| 3-sulfate | | BA Met. | | | | | | | | | |
| hexadecatrienoate | Lipid | Poly. | | | | | | | 0.03 | 0.03 | Low > Mod |
| (16:3n3) | | FA | | | | | | | | | |
| PAHSA (16:0/OH- | Lipid | Hydroxyl | | | | | | | 3.53E−03 | 0.03 | Low > Mod |
| 18:0) | | FA | | | | | | | | | |
| 4-hydroxyphenyl- | AA | Tyrosine | 0.02 | 0.03 | High > | | | | | | |
| pyruvate | | Met. | | | Low | | | | | | |
| 3-methyl-2- | AA | Leu, Ile, | 0.01 | 0.03 | High > | | | | | | |
| oxovalerate | | Val Met. | | | Low | | | | | | |
| 4-methyl-2- | AA | Leu, Ile, | 0.03 | 0.04 | High > | | | | | | |
| oxopentanoate | | Val Met. | | | Low | | | | | | |
| beta- | AA | Leu, Ile, | 0.06 | 0.06 | High > | | | | | | |
| hydroxyisovalerate | | Val Met. | | | Low | | | | | | |
| 2,3-dihydroxy-5- | AA | Met, Cys, | 0.05 | 0.05 | Low > | | | | | | |
| methylthio-4- | | SAM, | | | High | | | | | | |
| pentenoate (DMTPA) | | Ta Met. | | | | | | | | | |
| N-formylmethionine | AA | Met, Cys | 0.05 | 0.05 | Low > | | | | | | |
| | | SAM, | | | High | | | | | | |
| | | Ta Met. | | | | | | | | | |
| hydroxy-N6,N6,N6- | AA | Lys. Met. | | | | | | | 0.03 | 0.03 | Low > Mod |
| trimethyllysine | | | | | | | | | | | |
| l-methyladenosine | Nucleotide | Purine Met. | | | | 0.05 | 0.05 | Mod > | | | |
| | | | | | | | | High | | | |
| cytidine | Nucleotide | Pyrimidine | 0.02 | 0.03 | Low > | 0.01 | 0.03 | High > | | | |
| | | Met. | | | High | | | Mod | | | |
| digalacturonic acid | Xenobiotics | Food | | | | 0.04 | 0.05 | High > | | | |
| | | Component | | | | | | Mod | | | |
| 7-methylurate | Xenobiotics | Xanthine | | | | 0.03 | 0.05 | High > | | | |
| | | Met. | | | | | | Mod | | | |
| 3,7-dimethylurate | Xenobiotics | Xanthine | | | | 0.05 | 0.05 | High > | | | |
| | | Met. | | | | | | Mod | | | |
| 3-(3-hydroxy- | Xenobiotics | Xanthine | | | | 0.04 | 0.05 | Mod > | | | |
| phenyl)propionate | | Met. | | | | | | High | | | |
| 5-acetylamino-6- | Xenobiotics | Xanthine | | | | 0.03 | 0.05 | Mod > | 0.01 | 0.03 | Mod > Low |
| amino-3-methyluracil | | Met. | | | | | | High | | | |
| nicotinamide | Cofactors | Nic. Met. | | | | | | | 0.02 | 0.03 | Mod > Low |
| riboside | and Vitamins | | | | | | | | | | |
| xylose | Carbohydrate | Pentose | | | | | | | 0.02 | 0.03 | Low > Mod |
| | | Met. | | | | | | | | | |
| succinylcarnitine | Energy | TCA Cycle | | | | | | | 0.02 | 0.03 | Low > Mod |
| pentose acid* | Part. | Part. | | | | | | | 0.01 | 0.03 | Low > Mod |
| | Char. | Char. | | | | | | | | | |
| | Mol. | Mol. | | | | | | | | | |

All listed metabolites were q-significant when comparing between either high vs. low, high vs. moderate, or moderate vs. low physical activity individuals.
Int = Interpretation
p = p-value significant < 0.05
q = q-values derived from FDR correction, q-value significant < 0.05
AA = Amino Acid
And. Ster. = androgenic steroids
Met. = Metabolism
BA = Bile Acid
Leu, Ile, Val = Leucine, Isoleucine and Valine
Met, Cys, SAM, Tau = Methionine, Cysteine, SAM and Taurine
Lys. = Lysine
Nic. = Nicotinate and Nicotinamide
Poly. FA = Long Chain Polyunsaturated Fatty Acid (n3 and n6)
Hydroxyl FA = Fatty Acid Hydroxyl Fatty Acid
Part. Char. Mol. = Partially Characterized Molecules
Int = Interpretation
p = p-value significant < 0.05
q = q-values derived from FDR correction, q-value significant < 0.05
AA = Amino Acid
And. Ster. = androgenic steroids
Met. = Metabolism
BA = Bile Acid
Leu, Ile, Val = Leucine, Isoleucine and Valine
Met, Cys, SAM, Tau = Methionine, Cysteine, SAM and Taurine
Lys. = Lysine
Nic. = Nicotinate and Nicotinamide
Poly. FA = Long Chain Polyunsaturated Fatty Acid (n3 and n6)
Hydroxyl FA = Fatty Acid Hydroxyl Fatty Acid
Part. Char. Mol. = Partially Characterized Molecules

TABLE 5
Physical Activity Interacts with Psychosocial Variables, Gut
Microbiome, Fecal Metabolites, and Brain Connectivity.
High vs Low

| | | | | |
|---|---|---|---|---|
| | | Pearson | FDR | FDR |
| | | Correlation | Adjusted | Adjusted |
| Variable 1 | Variable 2 | P-value | P-value | for all |

Clinical Vs. Bacteria

| | | | | |
|---|---|---|---|---|
| BCope_Acceptance | Megasphaera | 0.01195511 | 0.01 | 0.01 |

Clinical Vs. Metabolite

| | | | | |
|---|---|---|---|---|
| BRS_Score | cytidine | 0.003596753 | 0.00 | 0.01 |

Metabolite Vs. Bacteria

| | | | | |
|---|---|---|---|---|
| X5alpha.androstan.3beta.17alpha.diol.monosulfate..1. | Methanobrevibacter | 0.003599801 | 0.01 | 0.01 |
| X5alpha.androstan.3beta.17alpha.diol.monosulfate..1. | Ruminiclostridium_6 | 0.001704317 | 0.01 | 0.01 |
| myo.inositol | Akkermansia | 0.004301153 | 0.01 | 0.01 |
| cytidine | Methanobrevibacter | 0.049060163 | 0.05 | 0.05 |

p-value significant < 0.05.
Brief Resilience Scale (BRS), Yale Food Addiction Scale (YFAS), Brief-COPE (BCope) Networks. SMN: sensorimotor, DMN: default mode, SAL: salience, CEN: central executive, CAN: central autonomic, ERN: emotion regulation, OCC: occipital.

Moderate vs Low

| | | | | |
|---|---|---|---|---|
| | | Pearson | FDR | FDR |
| | | Correlation | Adjusted | Adjusted |
| Variable 1 | Variable 2 | P-value | P-value | for all |

Brain vs. Clinical

| | | | | |
|---|---|---|---|---|
| CEN L_SupPL OCC L_Cun | YFAS_TimeSpent | 0.01788679 | 0.04 | 0.05 |
| DMN R_MTG DMN R_AngG | YFAS_Tolerance | 0.037676528 | 0.04 | 0.05 |
| DMN R_MTG DMN R_AngG | YFAS_ContinuedUse | 0.02302368 | 0.04 | 0.05 |
| DMN R_MTG DMN R_AngG | YFAS_LossControl | 0.027200312 | 0.04 | 0.05 |
| DMN R_MTG DMN R_AngG | YFAS_SymptomCount | 0.033752023 | 0.04 | 0.05 |

Brain Vs. Bacteria

| | | | | |
|---|---|---|---|---|
| CEN L_SupPL OCC L_Cun | Ruminococcaceae_UCG.014 | 0.045308278 | 0.05 | 0.05 |
| DMN R_MTG DMN R_AngG | Methanobrevibacter | 0.019226991 | 0.05 | 0.05 |
| DMN R_MTG DMN R_AngG | Ruminococcaceae_UCG.014 | 0.044524603 | 0.05 | 0.05 |

Brain Vs. Metabolite

| | | | | |
|---|---|---|---|---|
| DMN R_MTG DMN R_AngG | xylose | 0.033469218 | 0.04 | 0.05 |
| DMN R_MTG DMN R_AngG | succinylcarnitine..C4. | 0.043837943 | 0.04 | 0.05 |
| DMN R_MTG DMN R_AngG | pentose.acid. | 0.001113775 | 0.01 | 0.01 |
| DMN R_MTG DMN R_AngG | taurolithocholate.3.sulfate | 0.001996887 | 0.01 | 0.01 |
| DMN R_MTG DMN R_AngG | X5.acetylamino.6.amino.3.methyluracil | 0.030280597 | 0.04 | 0.05 |
| DMN R_MTG DMN R_AngG | X5.acetylamino.6.amino.3.methyluracil.1 | 0.030280597 | 0.04 | 0.05 |

Clinical Vs. Bacteria

| | | | | |
|---|---|---|---|---|
| YFAS_LossControl | Methanobrevibacter | 0.046792209 | 0.05 | 0.05 |

Clinical Vs. Metabolite

| | | | | |
|---|---|---|---|---|
| YFAS_Tolerance | nicotinamide riboside | 0.01352267 | 0.02 | 0.04 |
| YFAS_GivenUp | X5.acetylamino.6.amino.3.methyluracil | 0.000306266 | 0.00 | 0.00 |
| YFAS_GivenUp | nicotinamide.riboside | 0.012389067 | 0.02 | 0.04 |
| YFAS_GivenUp | X5.acetylamino.6.amino.3.methyluracil.1 | 0.000306266 | 0.00 | 0.00 |
| YFAS_TimeSpent | X5.acetylamino.6.amino.3.methyluracil | 0.049190769 | 0.05 | 0.05 |
| YFAS_TimeSpent | X5.acetylamino.6.amino.3.methyluracil.1 | 0.049190769 | 0.05 | 0.05 |
| YFAS_LossControl | X5.acetylamino.6.amino.3.methyluracil | 0.006061535 | 0.01 | 0.02 |
| YFAS_LossControl | X5.acetylamino.6.amino.3.methyluracil.1 | 0.006061535 | 0.01 | 0.02 |

Metabolite Vs. Bacteria

| | | | | |
|---|---|---|---|---|
| xylose | Megasphaera | 0.040524195 | 0.04 | 0.05 |
| xylose | Ruminiclostridium_6 | 0.031227707 | 0.04 | 0.05 |
| succinylcarnitine..C4. | Ruminococcaceae_UCG.014 | 0.040291291 | 0.04 | 0.05 |
| pentose.acid. | Ruminococcaceae_UCG.014 | 0.031999913 | 0.04 | 0.05 |
| taurochenodeoxycholate | Megasphaera | 1.22262E−30 | 0.00 | 0.00 |

High vs Moderate

| | | | | |
|---|---|---|---|---|
| | | Pearson | FDR | FDR |
| | | Correlation | Adjusted | Adjusted |
| Variable 1 | Variable 2 | P-value | P-value | for all |

Brain vs. Clinical

| | | | | |
|---|---|---|---|---|
| DMN R_SupTS DMN R_MTG | Education | 0.007582332 | 0.01 | 0.03 |
| DMN L_SupTS OCC L_AOcS | Education | 0.041635773 | 0.04 | 0.04 |
| CEN L_SbPS ERN L_InfFS | Education | 0.000602068 | 0.01 | 0.02 |
| DMN L_SupTS OCC L_SupOcS_TrOcS | Education | 0.002951103 | 0.01 | 0.02 |
| DMN R_MTG OCC L_SupOcS_TrOcS | Education | 0.032872458 | 0.04 | 0.04 |
| SMN R_SupFS CEN R_MFG | Education | 0.011149748 | 0.02 | 0.03 |
| ERN L_InfFS DMN L_AngG | Education | 0.027466985 | 0.03 | 0.04 |
| DMN R_SupTS ERN L_PaHipG | Education | 0.003427293 | 0.01 | 0.02 |
| OCC R_SupOcG DMN L_SupTS | Education | 0.002981899 | 0.01 | 0.02 |
| CEN L_MFG DMN L_PrCun | Education | 0.010588296 | 0.02 | 0.03 |
| CAN L_OrG DMN L_SupTS | Education | 0.001290338 | 0.01 | 0.02 |

Brain Vs. Metabolite

| | | | | |
|---|---|---|---|---|
| CEN R_SupPL DMN R_SuMarG | myo.inositol | 0.027357413 | 0.04 | 0.04 |
| CEN R_SupPL DMN R_SuMarG | digalacturonic.acid | 0.010833973 | 0.04 | 0.03 |
| CEN R_SupPL DMN R_SuMarG | chenodeoxycholic acid.sulfate..2. | 0.036421472 | 0.04 | 0.04 |
| CEN R_SupPL DMN R_SuMarG | cholate.sulfate | 0.044103405 | 0.04 | 0.04 |
| DMN R_MTG DMN L_TPl | digalacturonic.acid | 0.037825417 | 0.04 | 0.04 |
| DMN L_PosDCgG DMN L_InfTS | X3..3.hydroxyphenyl.propionate | 0.01049805 | 0.04 | 0.03 |
| CEN R_MFG DMN R_MTG | cytidine | 0.00768146 | 0.04 | 0.03 |
| SMN L_InfPrCS SMN L_PosCS | myo.inositol | 0.041374472 | 0.04 | 0.04 |
| DMN L_PrCun OCC L_LinG | digalacturonic.acid | 0.005525112 | 0.04 | 0.03 |
| DMNL_SupTS OCC L_SupOcS_TrOcS | X1.methyladenosine | 0.028540333 | 0.04 | 0.04 |
| OCC R_SupOcG CEN L_IntPS_TrPS | X5alpha.androstan.3beta.17alpha.diol.monosulfate..1. | 0.03747232 | 0.04 | 0.03 |
| OCC R_SupOcG CEN L_IntPS_TrPS | digalacturonic.acid | 0.026148487 | 0.04 | 0.04 |
| OCC R_SupOcG CEN L_IntPS_TrPS | chenodeoxycholic.acid.sulfate..2. | 0.032723815 | 0.04 | 0.04 |
| DMN R_AngG DMN R_MTG | X3..3.hydroxyphenyl.propionate | 0.017086755 | 0.04 | 0.03 |
| OCC R_SupOcG DMN L_PrCun | digalacturonic.acid | 0.026039454 | 0.04 | 0.04 |
| OCC R_SupOcG DMN L_MTG | digalacturonic.acid | 0.013681535 | 0.04 | 0.03 |
| DMN R_SupTGLp DMN R_MTG | cytidine | 0.044152392 | 0.04 | 0.04 |
| ERN L_ACgG_S CAN L_SbOrS | X7.methylurate | 0.001411606 | 0.03 | 0.02 |
| ERN L_ACgG_S CAN L_SbOrS | X7.methylurate.1 | 0.001411606 | 0.03 | 0.02 |
| ERN L_ACgG_S CAN L_SbOrS | X3.7.dimethylurate | 0.020173603 | 0.04 | 0.03 |
| DMN L_PrCun OCC L_CcS | X5alpha.androstan.3beta.17alpha.diol.monosulfate..1. | 0.018519332 | 0.04 | 0.03 |
| OCC R_MOcG OCC R_SupOcS_TrOcS | X7.methylurate | 0.012252008 | 0.04 | 0.03 |
| OCC R_MOcG OCC R_SupOcS_TrOcS | X7.methylurate.1 | 0.012252008 | 0.04 | 0.03 |
| OCC R_MOcG OCC R_SupOcS_TrOcS | X3.7.dimethylurate | 0.01510974 | 0.04 | 0.03 |
| SMN R_SupFG CAN R_OrG | X7.methylurate | 0.035630049 | 0.04 | 0.04 |
| SMN R_SupFG CAN R_OrG | X7.methylurate.1 | 0.035630049 | 0.04 | 0.04 |
| DMN R_PrCun OCC R_SupOcG | digalacturonic.acid | 0.005981647 | 0.04 | 0.03 |
| OCC R_Cun DMN L_PrCun | digalacturonic.acid | 0.033678022 | 0.04 | 0.04 |
| SAL R_MPosCgG_S SMN R_SbCG_S | X1.methyladenosine | 0.007293096 | 0.04 | 0.03 |
| DMN R_SupTS SMN L_SupFG | X3..3.hydroxyphenyl.propionate | 0.042821377 | 0.04 | 0.04 |
| CEN L_MFG SMN L_SupFG | X1.methyladenosine | 0.002756045 | 0.04 | 0.02 |
| DMN L_SupTGLp SMN L_SupFG | X1.methyladenosine | 0.013538973 | 0.04 | 0.03 |
| DMN R_MTG OCC L_SupOcS_TrOcS | X5.acetylamino.6.amino.3.methyluracil | 0.018952665 | 0.04 | 0.03 |
| DMN R_MTG OCC L_SupOcS_TrOcS | X5.acetylamino.6.amino.3.methyluracil.1 | 0.018952665 | 0.04 | 0.03 |
| DMN R_MTG OCC L_SupOcS_TrOcS | X1.methyladenosine | 0.016736511 | 0.04 | 0.03 |
| CEN R_SupPL OCC R_SupOcG | myo.inositol | 0.01788801 | 0.04 | 0.03 |
| CEN R_SupPL OCC R_SupOcG | digalacturonic acid | 0.028048531 | 0.04 | 0.04 |
| CEN R_MFG SMN R_PRCG | X1.methyladenosine | 0.037831325 | 0.04 | 0.04 |
| CEN R_MFG DMN R_CgSMarp | X1.methyladenosine | 0.028630549 | 0.04 | 0.04 |
| CEN R_MFG SMN R_PosCG | X1.methyladenosine | 0.006889775 | 0.04 | 0.03 |
| CAN L OrG DMN L_SupTS | cytidine | 0.024076239 | 0.04 | 0.04 |

Clinical vs Metabolite

| | | | | |
|---|---|---|---|---|
| Education | X3..3.hydroxyphenyl.propionate | 0.010419891 | 0.01 | 0.03 |

Networks: SMN=sensorimotor, DMN=default mode, SAL=salience, CEN=central executive, CAN=central autonomic, ERN=emotion regulation, OCC=occipital.
Brain regions: L_SupPL=Left Superior parietal lobule (lateral part of P1), L_Cun=Left Cuneus (O6), R_AngG=Right Angular gyrus, R_SupTS=Right Superior temporal sulcus (parallel sulcus), R_MTG=Right Middle temporal gyrus (T2), L_SupTS=Left Superior temporal sulcus (parallel sulcus), L_AOcS=Left Anterior occipital sulcus and preoccipital notch (temporo-occipital incisure), L_SbPS=Left Subparietal sulcus, L_InfFS=Left Inferior frontal sulcus, R_SupFS=Right Superior frontal sulcus, R_MFG=Right Middle frontal gyrus (F2), L_InfFS=Left Inferior frontal sulcus, L_AngG=Left Angular gyrus, L_PaHipG=Left Parahippocampal gyrus, parahippocampal part of the medial occipito-temporal gyrus, (T5), L_OrG=Left Orbital gyri, R_SuMarG=Right Supramarginal gyrus, R_SupPL=Right Superior parietal lobule (lateral part of P1), L_TP1=Left Planum temporale or temporal plane of the superior temporal gyrus, L_PosDCgG=Left Posterior-dorsal part of the cingulate gyrus (dPCC), L_InfTS=Left Inferior temporal sulcus, R_MFG=Right Middle frontal gyrus (F2), L_InfPrCS=Left Inferior part of theprecentral sulcus, L_PosCS=Left Postcentral sulcus, L_LinG=Left Lingual gyrus, ligual part of the medial occipito-temporal gyrus, (05), L_SupOcS_TrOcS=Left Superior occipital sulcus and transverse occipital sulcus, L_IntPS_TrPS=Left Intraparietal sulcus (interparietal sulcus) and transverse parietal sulci, R_AngG=Right Angular gyrus, R_SupOcG=Right Superior occipital gyrus (O1), L_PrCun=Left Precuneus (medial part of P1), L_MTG=Left Middle temporal gyrus (T2), R_SupTGLp=Right Lateral aspect of the superior temporal gyrus, L_SbOrS=Left Suborbital sulcus (sulcus rostrales, supraorbital sulcus), L_ACgG_S=Left Anterior part of the cingulate gyrus and sulcus (ACC), L_PrCun=Left Precuneus (medial part of P1), L_CcS=Left Calcarine sulcus, R_SupOcS_TrOcS=Right Superior occipital sulcus and transverse occipital sulcus, R_MOcG=Right Middle occipital gyrus (O2, lateral occipital gyrus), R_OrG=Right Orbital gyri, R_SupFG=Right Superior frontal gyrus (F1), R_PrCun=Right Precuneus (medial part of P1), R_SupOcG=Right Superior occipital gyrus (O1), R_Cun=Right Cuneus (O6), R_MPosCgG_S=Right Middle-posterior part of the cingulate gyrus and sulcus (pMCC), R_SbCG_S=Right Subcentral gyrus (central operculum) and sulci, L_MFG=Left Middle frontal gyrus (F2), L_SupTGLp=Left Lateral aspect of the superior temporal gyrus, L_SupFG=Left Superior frontal gyrus (F1), R_MTG=Right Middle temporal gyrus (T2), R_PRCG=Right Precentral gyrus, R_CgSMarp=Right Marginal branch (or part) of the cingulate sulcus, R_PosCG=Right Postcentral gyrus, L_SupTS=Left Superior temporal sulcus

### Example 2: Improved Psychosocial Measures Associated with Physical Activity May Be Explained by Alterations in Brain-Gut Microbiome Signatures; Identification of Agents and Metabolites Disclosed Herein

### Results

### Participant Characteristics and Psychosocial Measures

Psychosocial and behavioral characteristics of the 92 individuals (males=24, females=68) who are overweight or obese (mean BMI=33.22 kg/m², mean age=32.84 years) are summarized in Table 6 Based on the IPAQ scoring guidelines for determining physical activity (PA) levels, the average total PA in the high (n=43, males=15, females=28), moderate (n=32, males=5, females=27), and low (n=17, males=4, females=13) groups were 13,432.84 MVET minutes, 1,822.953 MVET minutes, and 5,081.70 MET minutes respectively (p<0.001). There were no significant differences in education or income levels between the groups, except within the high versus moderate PA comparison for education level (p=0.05).

TABLE 6
Participant's psychosocial characteristics with physical activity-based correlations.

| | | | | | | | | Moderate |
|---|---|---|---|---|---|---|---|---|
| | All (N = 92) | | | | High PA (N = 43) | | | PA (N = 32) |
| | Mean | SD | Range | N | Mean | SD | Range | Mean |
| Age | 32.84 | 10.30 | [18, 54] | 92 | 35.12 | 11.77 | [18, 54] | 29.65 |
| BMI | 33.22 | 4.54 | [25.32, 47.54] | 92 | 33.25 | 4.50 | [25.59, 45.29] | 33.98 |
| | EDUCATION AND SOCIOECONOMIC STATUS | | | | | | | |
| Education | 4.81 | 0.94 | [2, 6] | 89 | 4.60 | 0.94 | [2, 6] | 4.94 |
| Income | 6.55 | 2.46 | [1, 9] | 89 | 6.46 | 2.67 | [1, 9] | 6.73 |
| | PHYSICAL ACTIVITY (IPAQ) | | | | | | | |
| Total | 3935.06 | 4225.91 | [0, 26099.41] | 92 | 5952.78 | 4801.98 | [609, 26099.41) | 1107.60 |
| Walking | | | | | | | | |
| Total | 2508.86 | 3524.13 | [0, 21840] | 92 | 3870.01 | 4086.79 | [0, 21840] | 457.94 |
| Moderate | | | | | | | | |
| Total | 1938.87 | 4232.45 | [0, 32688] | 92 | 3610.02 | 5685.29 | [0, 32688] | 257.41 |
| Vigorous | | | | | | | | |
| Total | 8382.79 | 8936.20 | [0, 56507.11] | 92 | 13432.84 | 9488.34 | [3834, 56507.11) | 1822.95 |
| Physical | | | | | | | | |
| Activity | | | | | | | | |
| | RESILIENCE | | | | | | | |
| BRS | 22.681 | 4.718 | [9, 30] | 91 | 23.9286 | 4.8760 | [9, 30] | 21.059 |
| Score | | | | | | | | |
| | BRIEF COPE | | | | | | | |
| Self | 4.70 | 1.70 | [2, 8] | 88 | 4.56 | 1.66 | [2, 8] | 4.47 |
| Distraction | | | | | | | | |
| Active | 5.63 | 1.91 | [2, 8] | 88 | 5.78 | 1.98 | [2, 8] | 5.00 |
| Coping | | | | | | | | |
| Denial | 2.38 | 0.93 | [2, 8] | 87 | 2.25 | 0.67 | [2, 5] | 2.41 |
| Substance | 2.20 | 0.79 | [2, 8] | 88 | 2.29 | 1.05 | [2, 8] | 2.12 |
| Use | | | | | | | | |
| Emotional | 5.52 | 2.01 | [2, 8] | 88 | 5.54 | 2.12 | [2, 8] | 5.18 |
| Support | | | | | | | | |
| Intrumental | 5.41 | 2.07 | [2, 8] | 88 | 5.46 | 2.23 | [2, 8] | 4.65 |
| Support | | | | | | | | |
| Behavioral | 2.42 | 0.74 | [2, 5] | 88 | 2.37 | 0.70 | [2, 4] | 2.35 |
| Disengagemnet | | | | | | | | |
| Venting | 4.20 | 1.68 | [2, 8] | 87 | 4.07 | 1.63 | [2, 8] | 4.38 |
| Positive | 5.28 | 1.94 | [2, 8] | 88 | 5.46 | 1.92 | [2, 8] | 4.53 |
| Reframing | | | | | | | | |
| Planning | 5.84 | 1.73 | [2, 8] | 89 | 6.02 | 1.77 | [2, 8] | 5.18 |
| Humor | 4.30 | 1.77 | [2, 8] | 89 | 4.43 | 1.85 | [2, 8] | 3.76 |
| Acceptance | 5.72 | 1.81 | [2, 8] | 89 | 6.17 | 1.82 | [2, 8] | 5.06 |
| Religion | 4.51 | 2.20 | [2, 8] | 87 | 4.73 | 2.26 | [2, 8] | 3.53 |
| Self | 3.83 | 1.70 | [2, 8] | 89 | 3.95 | 1.86 | [2, 8] | 3.71 |
| Blame | | | | | | | | |
| | STRESS AND ANXIETY | | | | | | | |
| HAD | 5.12 | 3.71 | [0, 14] | 92 | 4.56 | 3.82 | [0, 14] | 5.18 |
| Anxiety | | | | | | | | |
| HAD | 2.46 | 2.60 | [0, 13] | 92 | 1.95 | 2.37 | [0, 13] | 3.06 |
| Depression | | | | | | | | |
| | FOOD CRAVINGS (Yale Food Addiction Scale) | | | | | | | |
| Withdrawal | 0.217 | 0.531 | [0, 3] | 92 | 0.16 | 0.43 | [0, 2] | 0.41 |
| Tolerance | 0.205 | 0.529 | [0, 2] | 88 | 0.20 | 0.51 | [0, 2] | 0.44 |
| Continued Use | 0.239 | 0.429 | [0, 1] | 92 | 0.19 | 0.39 | [0, 1] | 0.19 |
| Given Up | 0.165 | 0.601 | [0, 4] | 91 | 0.05 | 0.22 | [0, 1] | 0.59 |
| Time Spent | 0.304 | 0.588 | [0, 3] | 92 | 0.16 | 0.43 | [0, 2] | 0.82 |
| Loss Control | 0.110 | 0.379 | [0, 2] | 91 | 0.05 | 0.31 | [0, 2] | 0.35 |
| Unsuccessful | 1.716 | 0.909 | [0, 4] | 88 | 1.64 | 1.01 | [0, 4] | 1.88 |
| Cut Down | | | | | | | | |
| ClinSig | 0.120 | 0.415 | [0, 2] | 92 | 0.05 | 0.21 | [0, 1] | 0.24 |
| Impairment | | | | | | | | |
| Symptom Count | 1.924 | 1.477 | [0, 7] | 92 | 1.60 | 1.12 | [0, 5] | 3.18 |
| | BODY MASS (Bioimpedance Analysis) | | | | | | | |
| Fat Mass (%) | 35.42 | 8.09 | [3, 51.10] | 90 | 34.10 | 8.78 | [3, 50.10) | 36.84 |
| Lean Body | 64.57 | 8.09 | [48.90, 97] | 90 | 65.88 | 8.77 | (49.90, 97] | 63.17 |
| Mass (%) | | | | | | | | |
| Total Weight | 100.00 | 0.00 | [100, 100] | 78 | 100.00 | 0.00 | [100, 100] | 100.00 |
| (%) | | | | | | | | |

| | | | | | | High | Mod | High |
|---|---|---|---|---|---|---|---|---|
| | | | | | | vs. | vs. | vs. |
| | Moderate | | | | | Mod | Low | Low |
| | PA (N = 32) | | Low PA (N = 17) | | | PA | PA | PA |
| | SD | Range | Mean | SD | Range | p-value | | |
| Age | 8.67 | [19, 54] | 31.47 | 8.12 | [19, 53] | 0.14 | 0.48 | 0.08 |
| BMI | 4.33 | [25.32, 42.07] | 32.77 | 5.19 | [27.27, 47.54] | 0.65 | 0.39 | 0.59 |
| | EDUCATION AND SOCIOECONOMIC STATUS | | | | | | | |
| Education | 0.89 | [3, 6] | 5.03 | 0.97 | [3, 6] | 0.05 | 0.74 | 0.21 |
| Income | 2.07 | [1, 9] | 6.41 | 2.69 | [1, 9] | 0.89 | 0.90 | 1.00 |
| | PHYSICAL ACTIVITY (IPAQ) | | | | | | | |
| Total | 2985.03 | [0, 14157] | 2725.83 | 1189.69 | [0, 4223.71) | 1.29E−03 | 0.004 | 1.34E−04 |
| Walking | | | | | | | | |
| Total | 2880.03 | [0, 14669] | 1769.37 | 425.30 | [0, 1260] | 0.02 | 0.07 | 1.16E−03 |
| Moderate | | | | | | | | |
| Total | 1125.42 | [0, 5440] | 586.50 | 644.67 | [0, 2576] | 4.14E−03 | 0.27 | 0.02 |
| Vigorous | | | | | | | | |
| Total | 6364.02 | [772, 34266] | 5081.70 | 1824.55 | [0, 7516.71) | 5.09E−05 | 0.05 | 5.95E−06 |
| Physical | | | | | | | | |
| Activity | | | | | | | | |
| | RESILIENCE | | | | | | | |
| BRS | 4.596 | [13, 30] | 21.906 | 3.913 | [16, 29] | 0.07 | 0.52 | 0.04 |
| Score | | | | | | | | |
| | BRIEF COPE | | | | | | | |
| Self | 1.75 | [2, 8] | 5.03 | 1.70 | [2, 7] | 0.25 | 0.29 | 0.85 |
| Distraction | | | | | | | | |
| Active | 1.91 | [2, 8] | 5.77 | 1.73 | [2, 8] | 0.98 | 0.18 | 0.16 |
| Coping | | | | | | | | |
| Denial | 1.22 | [2, 8] | 2.53 | 0.87 | [2, 5] | 0.22 | 0.72 | 0.45 |
| Substance | 0.43 | [2, 4] | 2.13 | 0.49 | [2, 4] | 0.44 | 0.91 | 0.52 |
| Use | | | | | | | | |
| Emotional | 1.95 | [2, 8] | 5.70 | 1.91 | [2, 8] | 0.74 | 0.38 | 0.55 |
| Support | | | | | | | | |
| Intrumental | 1.94 | [2, 8] | 5.77 | 1.80 | [2, 8] | 0.55 | 0.06 | 0.19 |
| Support | | | | | | | | |
| Behavioral | 0.86 | [2, 5] | 2.53 | 0.61 | [2, 4] | 0.37 | 0.45 | 0.95 |
| Disengagemnet | | | | | | | | |
| Venting | 1.76 | [2, 8] | 4.27 | 1.75 | [2, 7] | 0.63 | 0.84 | 0.54 |
| Positive | 2.03 | [2, 8] | 5.47 | 1.70 | [2, 7] | 0.99 | 0.11 | 0.09 |
| Reframing | | | | | | | | |
| Planning | 1.59 | [2, 8] | 5.97 | 1.81 | [2, 8] | 0.89 | 0.13 | 0.10 |
| Humor | 1.77 | [2, 8] | 4.43 | 1.56 | [2, 6] | 0.99 | 0.20 | 0.20 |
| Acceptance | 1.74 | [2, 8] | 5.47 | 1.71 | [2, 8] | 0.11 | 0.44 | 0.04 |
| Religion | 2.23 | [2, 8] | 4.68 | 1.85 | [2, 8] | 0.92 | 0.09 | 0.07 |
| Self | 1.57 | [2, 8] | 3.73 | 1.57 | [2, 8] | 0.60 | 0.95 | 0.63 |
| Blame | | | | | | | | |
| | STRESS AND ANXIETY | | | | | | | |
| HAD | 3.61 | [0, 14] | 5.84 | 3.56 | [1, 13] | 0.14 | 0.54 | 0.57 |
| Anxiety | | | | | | | | |
| HAD | 2.56 | [0, 10] | 2.81 | 3.11 | [0, 10] | 0.14 | 0.77 | 0.14 |
| Depression | | | | | | | | |
| | FOOD CRAVINGS (Yale Food Addiction Scale) | | | | | | | |
| Withdrawal | 0.40 | [0, 1] | 0.19 | 0.87 | [0, 3] | 0.80 | 0.22 | 0.14 |
| Tolerance | 0.40 | [0, 2] | 0.10 | 0.73 | [0, 2] | 0.38 | 0.04 | 0.16 |
| Continued Use | 0.40 | [0, 1] | 0.47 | 0.51 | [0, 1] | 0.99 | 0.04 | 0.02 |
| Given Up | 0.39 | [0, 2] | 0.09 | 1.18 | [0, 4] | 0.52 | 0.03 | 0.01 |
| Time Spent | 0.42 | [0, 1] | 0.22 | 0.88 | [0, 3] | 0.58 | 2.07E−03 | 0.00 |
| Loss Control | 0.25 | [0, 1] | 0.06 | 0.61 | [0, 2] | 0.82 | 0.02 | 0.01 |
| Unsuccessful | 0.80 | [1, 4] | 1.72 | 0.86 | [1, 4] | 0.72 | 0.53 | 0.39 |
| Cut Down | | | | | | | | |
| ClinSig | 0.51 | [0, 2] | 0.16 | 0.56 | [0, 2] | 0.21 | 0.62 | 0.06 |
| Impairment | | | | | | | | |
| Symptom Count | 1.28 | [0, 6] | 1.69 | 1.98 | [1, 7] | 0.77 | 2.50E−03 | 2.52E−04 |
| | BODY MASS (Bioimpedance Analysis) | | | | | | | |
| Fat Mass (%) | 6.73 | (20.29, 51.10) | 36.43 | 8.56 | (14.10, 47.60) | 0.22 | 0.86 | 0.28 |
| Lean Body | 6.73 | (48.90, 79. 70) | 63.57 | 8.56 | (52.40, 85.91) | 0.23 | 0.86 | 0.28 |
| Mass (%) | | | | | | | | |
| Total Weight | 0.00 | [100, 100] | 100.00 | 0.00 | [100, 100] | 0.42 | 0.45 | 0.54 |
| (%) | | | | | | | | |

Means and standard deviations are reported for normally distributed data. P-significant<0.05. International PA Questionnaire (IPAQ), Brief Resilience Scale (BRS), Hospital Anxiety and Depression Scale (HADS), Yale Food Addiction Scale (YFAS), Brief-COPE (Bcope)

The high PA group had greater average BRS resilience scores (p=0.04) and ability to cope through acceptance of reality (p=0.04), and a significant difference compared to the low PA group. A trend in the anxiety scores were also seen with the HAD measures, with highest anxiety scores seen in the low PA groups, even though these did not reach significance.

Based on PA, there were also significant differences in multiple food addiction measures, as assessed using the Yale Food Addiction Scale (YFAS), with food craving scores being lowest with high PA group. When comparing between high vs. low PA groups, significant differences were found with the following YFAS measures: continued use (p=0.025), giving up (p=0.005), time spent (p<0.001), loss of control (p=0.01), and symptom count (p<0.001). Significant differences were also seen between moderate vs. low PA for the following YFAS measures: tolerance (p=0.04), continued use (p=0.04), time spent (p=0.002), loss of control (p=0.02), and symptom count (p=0.003; Table 6). There were no differences in macronutrient intake, including energy (kcal), fat (grams), carbohydrate (grams), protein (grams), and cholesterol (mg) when comparing between the PA level groups (Table 7).

TABLE 7
Participant's Macronutrient Intake Based on Physical Activity Level

| | | | | | | | Moderate |
|---|---|---|---|---|---|---|---|
| | All (N = 92) | | | High PA (N = 43) | | | PA (N = 32) |
| | Mean | SD | Range | Mean | SD | Range | Mean |
| ENERGY_KCAL | 2186.76 | 1459.01 | [524.41 | 2040.40 | 1185.87 | [524.41 | 2320.43 |
| (g) | | | 10998] | | | 6296.5] | |
| TOTAL_FAT | 91.14 | 61.36 | [19.86 | 86.57 | 50.96 | [26.15 | 95.10 |
| (g) | | | 399.36] | | | 238.34] | |
| CARBOHYDRATE | 254.04 | 187.54 | [49.93 | 230.15 | 147.45 | [49.93 | 277.30 |
| (g) | | | 1508.69] | | | 783.35] | |
| PROTEIN | 92.18 | 64.44 | [17.53 | 89.00 | 56.37 | [17.53 | 92.53 |
| (g) | | | 367.97] | | | 253.22] | |
| CHOLESTEROL | 342.51 | 254.40 | [17.13 | 339.22 | 246.37 | [65.1 | 336.04 |
| (mg) | | | 1366.41] | | | 1115.53] | |

| | | | | | | P-Values | | |
|---|---|---|---|---|---|---|---|---|
| | Moderate | | | | | High | Mod | High |
| | PA (N = 32) | | Low (N = 17) | | | vs. | vs. | vs. |
| | SD | Range | Mean | SD | Range | Mod | Low | Low |
| ENERGY_KCAL | 1892.40 | [830.85 | 2270.15 | 1117.63 | [772.44 | 0.71 | 0.99 | 0.85 |
| (g) | | 10998] | | | 5224.23] | | | |
| TOTAL_FAT | 74.86 | [19.86 | 94.10 | 58.32 | [25.49 | 0.84 | 1.00 | 0.91 |
| (g) | | 399.36] | | | 271.06] | | | |
| CARBOHYDRATE | 253.10 | [84.13 | 265.04 | 118.01 | [66.55 | 0.56 | 0.97 | 0.80 |
| (g) | | 1508.69] | | | 474.2] | | | |
| PROTEIN | 75.78 | [20.95 | 98.63 | 62.25 | [28.41 | 0.97 | 0.95 | 0.87 |
| (g) | | 367.97] | | | 286.81] | | | |
| CHOLESTEROL | 285.29 | [41.31 | 361.65 | 224.42 | [17.13 | 1.00 | 0.94 | 0.95 |
| (mg) | | 1366.41] | | | 949.12] | | | |

Means and standard deviations are reported for normally distributed data.
P-significant < 0.05.

### PA Differentiates Brain Functional Connectivity

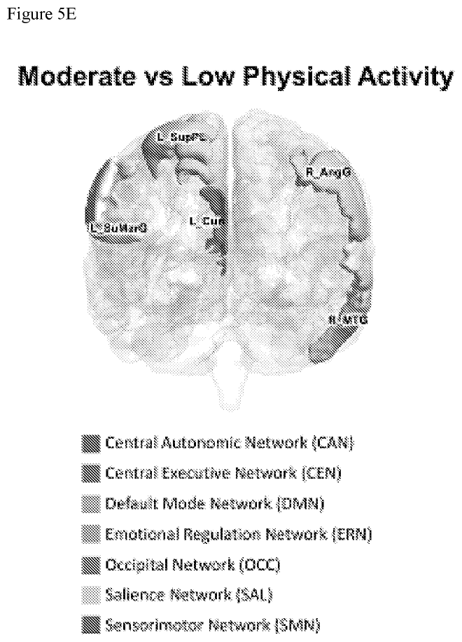

After adjusting for confounding variables such as age, sex, BMI, and diet, a sPLS-DA of brain functional connectivity displayed significant clustering based on PA level (FIG. 5A). Connectivity between 73 pairs of brain regions were associated with PA. The brain networks involved included the salience (SAL), central autonomic (CAN), central executive (CEN), emotional regulation (ERN), sensorimotor (SMN), default mode (DMN), and occipital (OCC) networks. The specific brain regions are summarized in Table 8.

TABLE 8
Functional Brain Connectivity Differences Based on Physical Activity.

| | | High | Moderate | High |
|---|---|---|---|---|
| Variable A | Variable B | vs. | vs. | vs. |

| | | | | | |
|---|---|---|---|---|---|
| Brain | Brain | VIP | Moderate | Low | Low |

| | | | | | | | |
|---|---|---|---|---|---|---|---|
| Network | Regions | Network | Regions | Loadings | Component 1 | Component 2 | Interpretation |

Brain Component 1

| | | | | | | | | | |
|---|---|---|---|---|---|---|---|---|---|
| SAL | R_MPosCgG_S | SMN | R_SbCG_S | −0.0632 | 19.1857 | 17.0948 | High ↑ | Low ↑ | High ↑ |
| CAN | L_OrG | DMN | L_SupTS | −0.0011 | 0.3274 | 0.2917 | High ↑ | Low ↑ | High ↑ |
| CEN | R_SupPL | CEN | R_SupPL | −0.0378 | 11.4683 | 10.2184 | High ↑ | Low ↑ | High ↑ |
| CEN | R_SupPL | DMN | R_SuMarG | −0.3676 | 111.6288 | 99.4632 | High ↑ | Low ↑ | High ↑ |
| CEN | L_SbPS | ERN | L_InfFS | −0.1571 | 47.7021 | 42.5034 | High ↑ | Low ↑ | High ↑ |
| CEN | R_POcS | OCC | R_SupOcG | −0.2137 | 64.9121 | 57.8378 | High ↑ | Low ↑ | High ↑ |
| CEN | L_MFG | SMN | L_SupFG | −0.0539 | 16.3791 | 14.594 | High ↑ | Low ↑ | High ↑ |
| CEN | L_MFG | SMN | L_SupFG | −0.0282 | 8.5706 | 7.6365 | High ↑ | Low ↑ | High ↑ |
| CEN | L_MFG | DMN | L_PrCun | −0.0029 | 0.8808 | 0.7848 | High ↑ | Low ↑ | High ↑ |
| CEN | R_MFG | SMN | R_PRCG | −0.0112 | 3.3957 | 3.0256 | High ↑ | Low ↑ | High ↑ |
| CEN | R_MFG | DMN | R_CgSMarp | −0.0077 | 2.351 | 2.0948 | High ↑ | Low ↑ | High ↑ |
| CEN | R_MFG | SMN | R_PosCG | −0.0066 | 2.0067 | 1.788 | High ↑ | Low ↑ | High ↑ |
| ERN | L_InfFS | DMN | L_AngG | −0.0143 | 4.3377 | 3.8649 | High ↑ | Low ↑ | High ↑ |
| SMN | R_SupFS | CEN | R_MFG | −0.0313 | 9.5085 | 8.4722 | High ↑ | Low ↑ | High ↑ |
| SMN | L_SupFS | OCC | L_AOcS | −0.0894 | 27.1541 | 24.1947 | High ↑ | Low ↑ | High ↑ |
| SMN | L_InfPrCS | SMN | L_PosCS | −0.1311 | 39.8096 | 35.471 | High ↑ | Low ↑ | High ↑ |
| SMN | R_SupFG | CEN | R_MFG | −0.113 | 34.3084 | 30.5694 | High ↑ | Mod ↑ | High ↑ |
| SMN | R_SupFG | CAN | R_OrG | −0.1971 | 59.8447 | 53.3226 | High ↑ | Low ↑ | High ↑ |
| SMN | R_SupFG | CAN | R_OrG | −0.0734 | 22.3036 | 19.8729 | High ↑ | Low ↑ | High ↑ |
| SMN | R_SupFG | SMN | R_PosCG | −0.0676 | 20.5292 | 18.2918 | High ↑ | Low ↑ | High ↑ |
| SMN | R_SupFG | CEN | R_MFG | −0.0313 | 9.5085 | 8.4722 | High ↑ | Low ↑ | High ↑ |
| DMN | L_PrCun | OCC | L_LinG | −0.1247 | 37.8643 | 33.7377 | High ↑ | Low ↑ | High ↑ |
| DMN | L_PrCun | OCC | L_CcS | −0.0778 | 23.6216 | 21.0472 | High ↑ | Low ↑ | High ↑ |
| DMN | L_PrCun | OCC | L_LinG | −0.0804 | 24.4251 | 21.7632 | High ↑ | Low ↑ | High ↑ |
| DMN | R_AngG | OCC | L_SupOcG | −0.0174 | 5.2722 | 4.6977 | High ↑ | Mod ↑ | High ↑ |
| DMN | R_PrCun | OCC | R_SupOcG | −0.3397 | 103.1674 | 91.9239 | High ↑ | Low ↑ | High ↑ |
| DMN | R_PrCun | OCC | R_SupOcG | −0.0691 | 20.989 | 18.7016 | High ↑ | Low ↑ | High ↑ |
| DMN | R_PrCun | OCC | R_SupOcG | −0.0262 | 7.9594 | 7.092 | High ↑ | Low ↑ | High ↑ |
| DMN | L_SupTS | OCC | L_AOcS | −0.1596 | 48.4782 | 43.1949 | High ↑ | Low ↑ | High ↑ |
| DMN | R_MTG | OCC | L_SupOcS_TrOcS | −0.0197 | 5.9976 | 5.344 | High ↑ | Low ↑ | High ↑ |
| DMN | L_SupTS | OCC | L_SupOcS_TrOcS | −0.1228 | 37.2835 | 33.2202 | High ↑ | Low ↑ | High ↑ |
| DMN | L_SupTGLp | SMN | L_SupFG | −0.0218 | 6.6263 | 5.9042 | High ↑ | Low ↑ | High ↑ |
| DMN | R_SuMarG | SMN | L_SupFG | −0.1168 | 35.4697 | 31.6041 | High ↑ | Low ↑ | High ↑ |
| DMN | R_SupTS | SMN | L_SupFG | −0.034 | 10.3189 | 9.1943 | High ↑ | Low ↑ | High ↑ |
| DMN | R_SupTS | DMN | R_MTG | −0.1921 | 58.3395 | 51.9814 | High ↑ | Low ↑ | High ↑ |
| DMN | R_SuMarG | DMN | R_MTG | −0.3894 | 118.2541 | 105.3663 | High ↑ | Mod ↑ | High ↑ |
| DMN | L_SupTS | DMN | L_TP1 | −0.0598 | 18.1718 | 16.1913 | High ↑ | Low ↑ | High ↑ |
| DMN | R_SupTGLp | DMN | R_MTG | −0.1057 | 32.1068 | 28.6077 | High ↑ | Low ↑ | High ↑ |
| DMN | R_SupTS | DMN | R_AngG | −0.0438 | 13.3139 | 11.8629 | High ↑ | Low ↑ | High ↑ |
| DMN | R_MTG | DMN | L_TP1 | −0.1962 | 59.5757 | 53.083 | High ↑ | Low ↑ | High ↑ |
| DMN | R_MTG | DMN | L_TP1 | −0.1072 | 32.5493 | 29.002 | High ↑ | Low ↑ | High ↑ |
| DMN | R_MTG | DMN | R_SuMarG | −0.0267 | 8.1115 | 7.2275 | High ↑ | Low ↑ | High ↑ |
| DMN | R_MTG | DMN | L_SupTGLp | −0.0286 | 8.6991 | 7.7511 | High ↑ | Low ↑ | High ↑ |
| OCC | R_Cun | DMN | L_PrCun | −0.0658 | 19.9697 | 17.7933 | High ↑ | Low ↑ | High ↑ |
| OCC | R_LinG | DMN | L_PrCun | −0.027 | 8.1917 | 7.2989 | High ↑ | Low ↑ | High ↑ |
| OCC | R_SupOcG | CEN | L_IntPS_TrPS | −0.1175 | 35.6806 | 31.792 | High ↑ | Low ↑ | High ↑ |
| OCC | R_SupOcG | CEN | R_POcS | −0.0881 | 26.7565 | 23.8405 | High ↑ | Mod ↑ | High ↑ |
| OCC | R_SupOcG | DMN | L_PrCun | −0.1122 | 34.0694 | 30.3564 | High ↑ | Low ↑ | High ↑ |
| OCC | R_SupOcG | DMN | L_MTG | −0.111 | 33.6996 | 30.0269 | High ↑ | Low ↑ | High ↑ |
| OCC | R_SupOcG | CEN | R_POcS | −0.111 | 33.6996 | 30.0269 | High ↑ | Low ↑ | High ↑ |
| OCC | R_SupOcG | OCC | R_Cun | −0.0516 | 15.6659 | 13.9586 | High ↑ | Low ↑ | High ↑ |
| OCC | R_SupOcG | CEN | L_POcS | −0.0168 | 5.1025 | 4.5464 | High ↑ | Low ↑ | High ↑ |
| OCC | R_SupOcG | OCC | L_Cun | −0.0242 | 7.3584 | 6.5565 | High ↑ | Low ↑ | High ↑ |
| OCC | R_SupOcG | DMN | L_SupTS | −0.005 | 1.5092 | 1.3447 | High ↑ | Low ↑ | High ↑ |
| OCC | L_Cun | OCC | L_LinG | −0.047 | 14.275 | 12.7192 | High ↑ | Low ↑ | High ↑ |
| CEN | R_MFG | CEN | R_IntPS_TrPS | 0.1601 | 48.6246 | 43.3253 | Mod ↑ | Mod ↑ | Low ↑ |
| CEN | R_MFG | DMN | R_MTG | 0.1456 | 44.213 | 39.3946 | Mod ↑ | Mod ↑ | Low ↑ |
| CEN | R_SupPL | OCC | R_SupOcG | 0.0287 | 8.713 | 7.7634 | Mod ↑ | Mod ↑ | Low ↑ |
| CEN | R_SupPL | OCC | R_SupOcG | 0.0131 | 3.983 | 3.5489 | Mod ↑ | Mod ↑ | Low ↑ |
| ERN | R_ACgG_S | CAN | L_SbOrS | 0.0242 | 7.36 | 6.5579 | Mod ↑ | Low ↑ | Low ↑ |
| ERN | L_ACgG_S | CAN | L_SbOrS | 0.0912 | 27.699 | 24.6803 | Mod ↑ | Mod ↑ | Low ↑ |
| DMN | R_SupTS | DMN | R_SupTS | 0.0279 | 8.4656 | 7.543 | Mod ↑ | Mod ↑ | Low ↑ |
| DMN | R_SupTS | DMN | R_SupTS | 0.0212 | 6.4297 | 5.7289 | Mod ↑ | Mod ↑ | Low ↑ |
| DMN | R_MTG | DMN | L_AngG | 0.0139 | 4.223 | 3.7628 | Mod ↑ | Low ↑ | Low ↑ |
| DMN | R_AngG | DMN | R_MTG | 0.116 | 35.2387 | 31.3983 | v | Mod ↑ | Low ↑ |
| DMN | L_PosDCg | DMN | L_InfTS | 0.1524 | 46.2822 | 41.2382 | Mod ↑ | Mod ↑ | Low ↑ |
| DMN | R_MTG | CEN | R_IntPS_TrPS | 0.2021 | 61.3663 | 54.6784 | Mod ↑ | Mod ↑ | Low ↑ |
| DMN | R_SupTS | ERN | L_PaHipG | 0.0141 | 4.286 | 3.8189 | Mod ↑ | Mod ↑ | Low ↑ |
| OCC | R_MOcG | OCC | R_SupOcS_TrOcS | 0.075 | 22.7807 | 20.2979 | Mod ↑ | Mod ↑ | Low ↑ |
| OCC | R_InfOcG_S | OCC | L_SupOcG | 0.1242 | 37.7238 | 33.6126 | Mod ↑ | Mod ↑ | Low ↑ |

Brain Component 2

| | | | | | | | | |
|---|---|---|---|---|---|---|---|---|
| CEN | L_SupPL | OCC | L_Cun | −0.948 | 130.7092 | Mod ↑ | Mod ↑ | High ↑ |
| DMN | R_MTG | DMN | R_AngG | 0.2826 | 38.964 | Mod ↑ | Low ↑ | Low ↑ |
| DMN | L_SuMarG | CEN | L_SupPL | 0.1461 | 20.1381 | High ↑ | Low ↑ | Low ↑ |

VIP=variable importance projection.
Comparisons of brain connectivity between each pair of brain regions are made between high vs. moderate, moderate vs. low, and high vs. low PA.
Networks. SMN: sensorimotor, DMN: default mode, SAL: salience, CEN: central executive, CAN: central autonomic, ERN: emotion regulation, OCC: occipital.
Brain regions: R_MposCgG_S=Right Middle-posterior part of the cingulate gyrus and sulcus (pMCC), L_OrG=Left Orbital gyri, R_SupPL=Right Superior parietal lobule (lateral part of P1), L_SbPS=Left Subparietal sulcus, R_PocS=Right Parieto-occipital sulcus (or fissure), L_MIFG=Left Middle frontal gyrus (F2), R_AcgG_S=Right Anterior part of the cingulate gyrus and sulcus (ACC), L_AcgG_S=Left Anterior part of the cingulate gyrus and sulcus (ACC), L_InfFS=Left Inferior frontal sulcus, R_SupFS=Right Superior frontal sulcus, L_SupFS=Left Superior frontal sulcus, L_InfPrCS=Left Inferior part of the precentral sulcus, R_SupFG=Right Superior frontal gyrus (F1), L_PrCun=Left Precuneus (medial part of P1), R_PrCun=Right Precuneus (medial part of P1), L_SupTS=Left Superior temporal sulcus (parallel sulcus), R_SupTS=Right Superior temporal sulcus (parallel sulcus), R_SupTGLp=Right Lateral aspect of the superior temporal gyms, R_AngG=Right Angular gyrus, L_PosDCgG=Left Posterior-dorsal part of the cingulate gyrus (dPCC), R_Cun=Right Cuneus (O6), R_LinG=Right Lingual gyms, lingual part of the medial occipito-temporal gyrus, (O5), R_MocG=Right Middle occipital gyrus (O2, lateral occipital gyrus), R_InfOcG_S=Right Inferior occipital gyrus (O3) and sulcus, L_SuMarG=Left Supramarginal gyms, R_SbCG_S=Right Subcentral gyrus (central operculum) and sulci, R_SupOcG=Right Superior occipital gyrus (O1), R_PRCG=Right Precentral gyrus, R_CgSMarp=Right Marginal branch (or part) of the cingulate sulcus, L_SbOrS=Left Suborbital sulcus (sulcus rostrales, supraorbital sulcus), R_MFG=Right Middle frontal gyrus (F2), L_AocS=Left Anterior occipital sulcus and preoccipital notch (temporo-occipital incisure), L_PosCS=Left Postcentral sulcus, R_OrG=Right Orbital gyri, R_PosCG=Right Postcentral gyrus, L_CcS=Left Calcarine sulcus, L_LinG=Left Lingual gyrus, lingual part of the medial occipito-temporal gyrus, (O5), L_SupOcG=Left Superior occipital gyrus (O1), L_SupOcS_TrOcS=Left Superior occipital sulcus and transverse occipital sulcus, L_SupFG=Left Superior frontal gyrus (F1), R_MTG=Right Middle temporal gyrus (T2), L_TP1=Left Planum temporale or temporal plane of the superior temporal gyrus, R_SuMarG=Right Supramarginal gyrus, L_SupTGLp=Left Lateral aspect of the superior temporal gyrus, L_AngG=Left Angular gyrus, L_InfTS=Left Inferior temporal sulcus, R_IntPS_TrPS=Right Intraparietal sulcus (interparietal sulcus) and transverse parietal sulci, L_PaHipG=Left Parahippocampal gyrus, parahippocampal part of the medial occipito-temporal gyrus, (T5), L_IntPS_TrPS=Left Intraparietal sulcus (interparietal sulcus) and transverse parietal sulci, L_MTG=Left Middle temporal gyrus (T2), L_PocS=Left Parieto-occipital sulcus (or fissure), L_Cun=Left Cuneus (O6), R_SupOcS_TrOcS=Right Superior occipital sulcus and transverse occipital sulcus, L_SupPL=Left Superior parietal lobule (lateral part of P1).

Compared to both moderate and low PA individuals, those with high PA have increased functional connectivity in 56 pairs of brain connections as summarized in Table 8, involving the DMN, CEN, SMN, OCC, CAN, ERN, and SAL networks (FIG. 5B, 5D). In contrast to high PA participants, those with moderate and low PA had significantly increased functional connectivity in 16 pairs of brain regions, including brain regions involving the DMN, CEN, OCC, ERN, and CAN networks.

When comparing moderate versus low PA, there were 55 pairs of brain connections that were increased in connectivity in the low PA group, involving the networks DMN, ERN, OCC, CEN, SAL, SMN, and CAN (FIG. 5C, 5E). Of these brain connections, 51 of these were the same regions that were increased in the high PA group when compared to the moderate and low groups, meaning that these regions were highest in functional connectivity in the high PA group, followed by the low PA individuals, and had the least functional connectivity in the moderate PA group (Table 8).

### PA and Gut Microbiome Composition

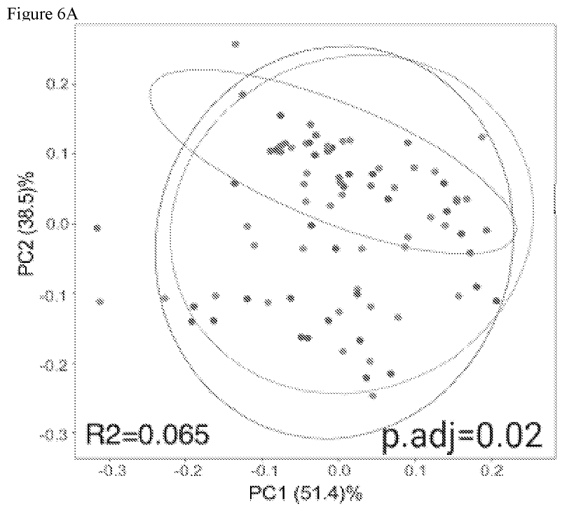

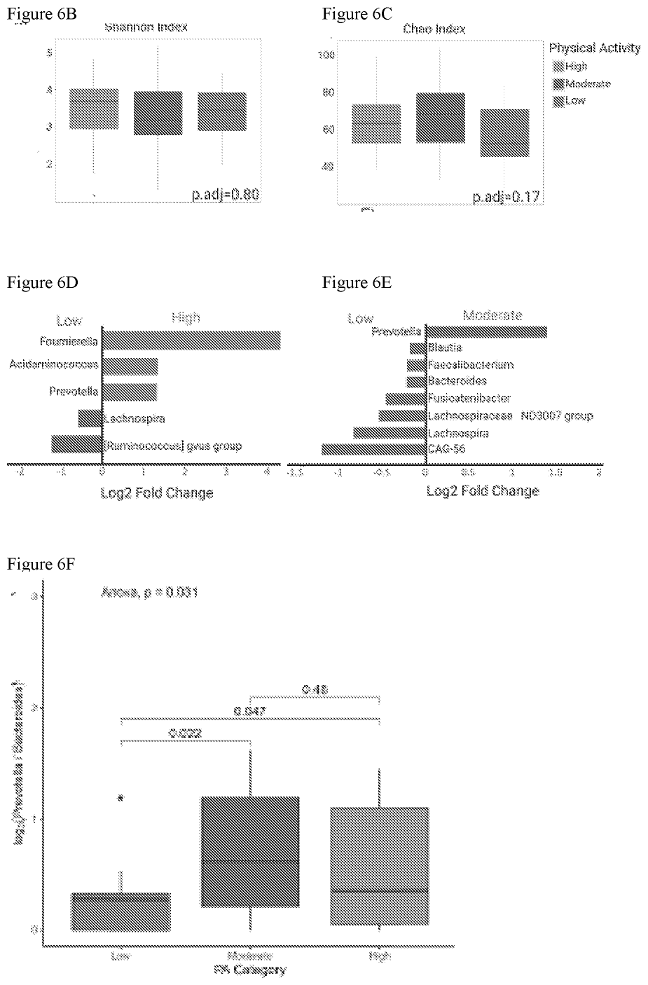

When comparing the three PA groups after adjusting for covariates such as age, sex, BMI and diet, significant differences in beta diversity were seen. The low PA group had a significantly different beta diversity compared to the high and moderate group, which had similar beta diversity signatures (FIG. 6A). No differences were seen with alpha-diversity indices (FIG. 6B, 6C). Significant differences in relative abundance were also seen when comparing both the high versus low and moderate versus low PA groups with the MaAslin2 analysis, after adjusting for covariates. When comparing high and low PA participants as seen in FIG. 6D, three genera (Fournierella, Acidaminococcus, and Prevotella) were higher in abundance and two genera (Lachnospira, Riminococcus gnavus) were lower in abundance in the high PA group. Fournierella demonstrated the greatest positive fold change when comparing high versus low PA. In the moderate versus low comparison, one genus (Prevotella) showed a greater relative abundance and seven genera (Blautia, Faecalibacterium, Bacteroides, Fusicantenibacter, Lachnospiraceae, Lachnospira, and CAG-56) had a lower relative abundance in the moderate compared to low PA (FIG. 6E). Prevotella showed the greatest positive fold change in the moderate versus low comparison and is increased in relative abundance in a dose-dependent fashion, as it also demonstrated a positive fold change in the high PA group compared to low (FIG. 6D, 6E).

When comparing Prevotella to Bacteroides ratio, there was overall significant differences seen across all PA groups (p=0.03). Specifically, there was a significant difference seen between the high vs. low PA groups (p=0.05) and the moderate vs. low (p=0.02), but not in the high vs. low (p=0.48) comparison. Individuals in the moderate PA group had the highest Prevotella to Bacteroides ratio and those in the low PA group had the lowest (FIG. 6F).

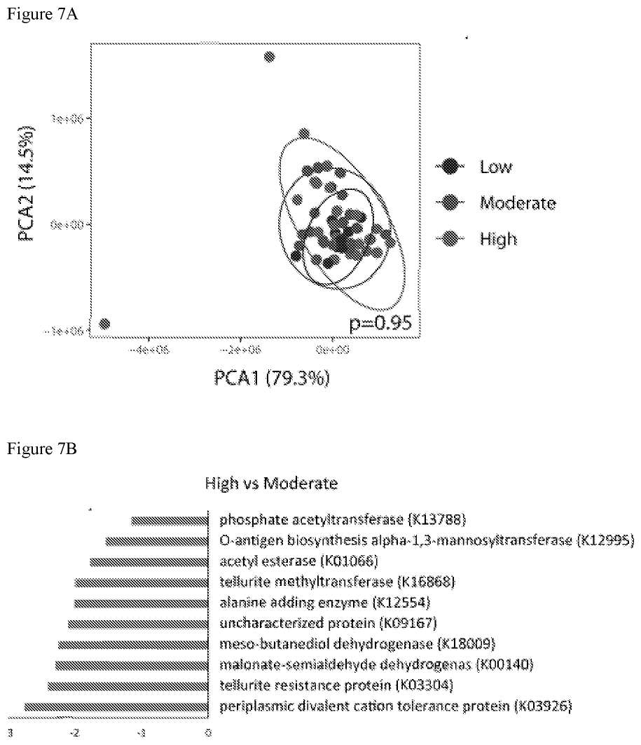

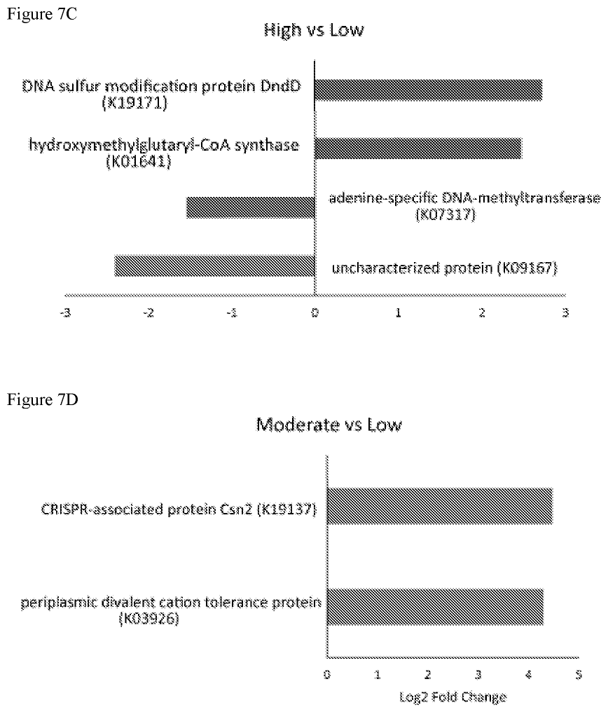

Microbial function was assessed by bacterial transcript abundances, which were annotated by KEGG orthology (KO)), and differential abundance testing identified 12 bacterial transcripts that were increased in relative abundance in the low PA group when compared with both the high and low PA groups, which is summarized in FIG. 7.

Fecal Metabolites Associated with PA

After adjusting for confounding variables such as age, sex, BMI, and diet, 32 metabolites were associated with PA, with 13 categorized as amino acids, seven as lipids, four as nucleotides, three as carbohydrates, two as peptides, two as cofactors, and one as belonging to the energy super pathway (Table 9).

TABLE 9
Fecal metabolites Associated with Physical Activity Levels.

| Component 1 Metabolites | | | | | | Interpretation | | | |
|---|---|---|---|---|---|---|---|---|---|
| | | | Loadings | Loadings | VIP | | High | Mod | High |
| | Super | | Component | Component | Component | VIP | vs. | vs. | vs. |
| Metabolite | Pathway | Sub Pathway | 1 | 2 | 1 | Component 2 | Mod | Low | Low |
| Cytosine | Nucleotide | Pyrimidine | 0.237 | 0.237 | 5.6803 | 3.2177 | Mod ↑ | Mod ↑ | High ↑ |
| | | Metabolism, | | | | | | | |
| | | Cytidine | | | | | | | |
| | | containing | | | | | | | |
| Glycosyl Ceramide | Lipid | Hexosylceramides | −0.1772 | 0.1772 | 4.4038 | 3.059 | Mod ↑ | Mod ↑ | Low ↑ |
| (D18:2/24:1, | | (HCER) | | | | | | | |
| D18:1/24:2) | | | | | | | | | |
| Histidine | Amino | Histidine | −0.1663 | 0.1663 | 4.184 | 2.7039 | Mod ↑ | Mod ↑ | High ↑ |
| | Acid | Metabolism | | | | | | | |
| Docosapentaenoate | Lipid | Hexosylceramides | −0.1437 | 0.1437 | 3.7006 | 2.6406 | High ↑ | Low ↑ | Low ↑ |
| (n6 DPA; 22:5n6) | | (HCER) | | | | | | | |
| Glycylvaline | Peptide | Dipeptide | −0.1382 | 0.1382 | 3.5839 | 2.5907 | Mod ↑ | Mod ↑ | High ↑ |
| Tyrosine | Amino | Tyrosine | −0.1357 | 0.1357 | 3.5245 | 2.5341 | Mod ↑ | Mod ↑ | High ↑ |
| | Acid | Metabolism | | | | | | | |
| Proline | Amino | Urea cycle; | −0.1307 | 0.1307 | 3.4315 | 2.4611 | Mod ↑ | Mod ↑ | High ↑ |
| | Acid | Arginine and | | | | | | | |
| | | Proline | | | | | | | |
| | | Metabolism | | | | | | | |
| Methionine | Amino | Methionine, | −0.129 | 0.129 | 3.3683 | 2.713 | Mod ↑ | Low ↑ | Low ↑ |
| Sulfoxide | Acid | Cysteine, SAM | | | | | | | |
| | | and Taurine | | | | | | | |
| | | Metabolism | | | | | | | |
| Hypoxanthine | Nucleotide | Purine | 0.1289 | 0.1289 | 3.3623 | 2.3695 | High ↑ | Mod ↑ | High ↑ |
| | | Metabolism, | | | | | | | |
| | | (Hypo)Xanthine/ | | | | | | | |
| | | Inosine | | | | | | | |
| | | containing | | | | | | | |
| Glycosyl-N-stearoyl- | Lipid | Hexosylceramides | −0.1223 | 0.1223 | 3.2429 | 2.4147 | High ↑ | Low ↑ | LLow ↑ |
| sphingosine | | (HCER) | | | | | | | |
| (d18:1/18:0) | | | | | | | | | |
| Thymine | Nucleotide | Pyrimidine | 0.1222 | 0.1222 | 3.2348 | 2.3544 | High ↑ | Mod ↑ | High ↑ |
| | | Metabolism, | | | | | | | |
| | | Thymine | | | | | | | |
| | | containing | | | | | | | |
| Homocitrulline | Amino | Urea cycle; | −0.1216 | 0.1216 | 3.2223 | 2.4064 | Mod ↑ | Low ↑ | Low ↑ |
| | Acid | Arginine and | | | | | | | |
| | | Proline | | | | | | | |
| | | Metabolism | | | | | | | |
| Maltose | Carbohydrate | Glycogen | 0.1202 | 0.1202 | 3.1837 | 2.2996 | Mod ↑ | Mod ↑ | Low ↑ |
| | | Metabolism | | | | | | | |
| N-Acetyl-Beta- | Carbohydrate | Aminosugar | −0.1175 | 0.1175 | 3.1472 | 2.3504 | High ↑ | Low ↑ | High ↑ |
| Glucosaminylamine | | Metabolism | | | | | | | |
| Ribulose/Xylulose | Carbohydrate | Pentose | 0.1164 | 0.1164 | 3.12 | 2.2495 | High ↑ | Mod ↑ | High ↑ |
| | | Metabolism | | | | | | | |
| N-Acetyl-1- | Amino | Histidine | −0.1142 | 0.1142 | 3.0787 | 2.4604 | Mod ↑ | Mod ↑ | High ↑ |
| Methylhistidine | Acid | Metabolism | | | | | | | |
| Serine | Amino | Glycine, Serine | −0.1141 | 0.1141 | 3.0786 | 2.3618 | Mod ↑ | Mod ↑ | High ↑ |
| | Acid | and Threonine | | | | | | | |
| | | Metabolism | | | | | | | |
| Leucine | Amino | Leucine, | −0.1138 | 0.1138 | 3.0722 | 2.1918 | Mod ↑ | Mod ↑ | High ↑ |
| | Acid | Isoleucine and | | | | | | | |
| | | Valine | | | | | | | |
| | | Metabolism | | | | | | | |
| Arachidoylcarnitine | Lipid | Fatty Acid | −0.1109 | 0.1109 | 2.9997 | 2.1966 | Mod ↑ | Mod ↑ | Low ↑ |
| (C20) | | Metabolism | | | | | | | |
| | | (Acyl | | | | | | | |
| | | Carnitine, | | | | | | | |
| | | Long Chain | | | | | | | |
| | | Saturated) | | | | | | | |
| 2′-Deoxyguanosine | Nucleotide | Purine | −0.1107 | 0.1107 | 3.0064 | 2.3525 | High ↑ | Low ↑ | Low ↑ |
| | | Metabolism, | | | | | | | |
| | | Guanine | | | | | | | |
| | | containing | | | | | | | |
| Phenylalanine | Amino | Phenylalanine | −0.1096 | 0.1096 | 2.9798 | 2.2345 | High ↑ | Mod ↑ | High ↑ |
| | Acid | Metabolism | | | | | | | |
| Aspartate | Amino | Alanine and | −0.1086 | 0.1086 | 2.9638 | 2.1294 | High ↑ | Mod ↑ | High ↑ |
| | Acid | Aspartate | | | | | | | |
| | | Metabolism | | | | | | | |
| Lignoceroylcarnitine | Lipid | Fatty Acid | −0.1061 | 0.1061 | 2.9143 | 2.0832 | Mod ↑ | Mod ↑ | High ↑ |
| (C24) | | Metabolism | | | | | | | |
| | | (Acyl | | | | | | | |
| | | Carnitine, | | | | | | | |
| | | Long Chain | | | | | | | |
| | | Saturated) | | | | | | | |
| Biocytin | Cofactors | Biotin | −0.104 | 0.104 | 2.8511 | 2.0857 | Mod ↑ | Mod ↑ | Low ↑ |
| | and Vitamins | Metabolism | | | | | | | |
| Glycosyl-N-(2- | Lipid | Hexosylceramides | −0.1038 | 0.1038 | 2.8546 | 2.1456 | Mod ↑ | Low ↑ | Low ↑ |
| Hydroxynervonoyl)- | | (HCER) | | | | | | | |
| Sphingosine | | | | | | | | | |
| (D18:1/24:1(2Oh)) | | | | | | | | | |
| Stearoylcarnitine | Lipid | Fatty Acid | −0.1036 | 0.1036 | 2.8622 | 2.0504 | High ↑ | Low ↑ | Low ↑ |
| (C18) | | Metabolism | | | | | | | |
| | | (Acyl | | | | | | | |
| | | Carnitine, | | | | | | | |
| | | Long Chain | | | | | | | |
| | | Saturated) | | | | | | | |
| Glycylisoleucine | Peptide | Dipeptide | −0.1015 | 0.1015 | 2.8063 | 2.0515 | High ↑ | Mod ↑ | High ↑ |
| Taurolithocholate | Lipid | Secondary Bile | −0.1013 | 0.1013 | 2.8077 | 2.2574 | Mod ↑ | Mod ↑ | Low ↑ |
| | | Acid | | | | | | | |
| | | Metabolism | | | | | | | |
| Threonine | Amino | Glycine, Serine | −0.0985 | 0.0985 | 2.7493 | 2.2574 | Mod ↑ | Mod ↑ | Low ↑ |
| | Acid | and Threonine | | | | | | | |
| | | Metabolism | | | | | | | |

Component 2 Metabolites

| | | | | | | | | |
|---|---|---|---|---|---|---|---|---|
| Succinate | Energy | TCA Cycle | −0.3954 | 0.2777 | 2.853 | High ↑ | Low ↑ | High ↑ |
| 1- | Cofactors | Nicotinate and | −0.3399 | 0 | 2.6557 | Mod ↑ | Mod ↑ | Low ↑ |
| Methylnicotinamide | and | Nicotinamide | | | | | | |
| | Vitamins | Metabolism | | | | | | |
| Argininate | Amino | Urea cycle; | −0.3284 | 1.3653 | 2.6563 | Mod ↑ | Mod ↑ | High ↑ |
| | Acid | Arginine and | | | | | | |
| | | Proline | | | | | | |
| | | Metabolism | | | | | | |
| Tyramine | Amino | Tyrosine | −0.3079 | 0.4866 | 2.5649 | High ↑ | Low ↑ | High ↑ |
| | Acid | Metabolism | | | | | | |
| Hyocholate | Lipid | Secondary Bile | −0.2755 | 0 | 2.4631 | High ↑ | Low ↑ | Low ↑ |
| | | Acid Metabolism | | | | | | |

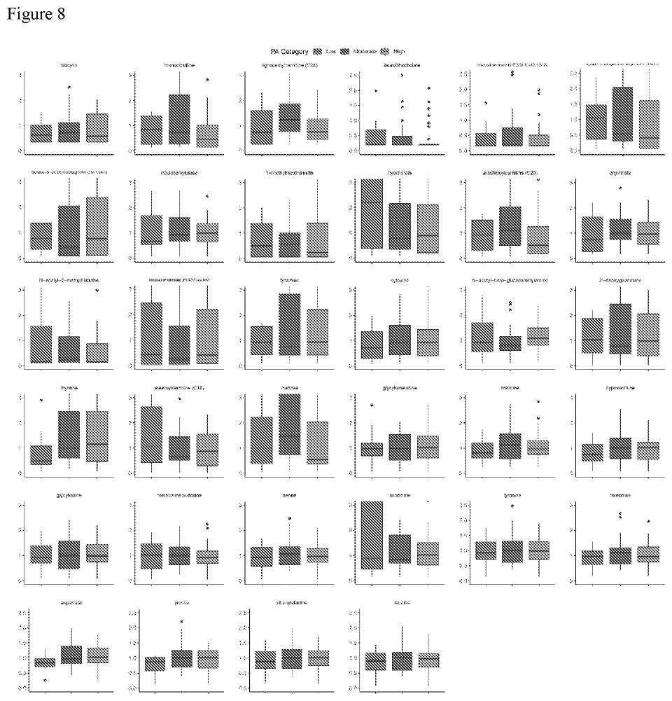

The top three metabolites that were most associated with PA were cytosine, glycosyl ceramide (D18:2/24:1, D18:1/24:2), and histidine. Additionally, lignoceroylcarnitine (C24) levels were highest in the moderate PA group and also elevated in the high PA group when compared to the low PA group. Glycosyl-N-(2-hydroxynervonoyl)sphingosine (D18:1/24:1 (20 h)) and 1-methylnicotinamide showed a dose dependent negative trend with more PA, with highest levels in the low PA group and lowest in those with high PA. On the other hand, ribulose, phenylalanine, aspartate, thymine, hypoxanthine, and glycylisoleucine levels showed a positive trend with PA, with highest levels in the high PA group. A summary of the trends for each metabolite that were associated with PA is shown in FIG. 8.

Significant associations were also identified between the significant psychosocial variables (coping, resilience score, food addiction measures, education), the metabolites, and pairs of connected brain regions across all physical activity group comparisons, which is summarized in Table 10.

TABLE 10
Physical Activity Interacts with Psychosocial Variables, Gut Microbiome, Fecal Metabolites, and Brain Connectivity.
High Physical Activity vs. Low Physical Activity
Brain vs Clinical

| Variable 1 | Variable 2 | r | p | p-adjusted | FDR |
|---|---|---|---|---|---|
| DMN R_SupTS DMN R_SupTS | BCope_Acceptance | −0.4649 | 0.0006 | 0.0233 | 0.0260 |
| DMN R_MTG OCC L_SupOcS_TrOcS | BCope_Acceptance | 0.3658 | 0.0083 | 0.0397 | 0.0377 |
| ERN L_InfFS DMN L_AngG | YFAS_ContinuedUse | −0.3752 | 0.0061 | 0.0375 | 0.0359 |
| DMN R_SupTS DMN R_MTG | YFAS_ContinuedUse | −0.3679 | 0.0073 | 0.0394 | 0.0362 |
| DMN R_SupTS DMN R_MTG | YFAS_SymptomCount | −0.3889 | 0.0044 | 0.0375 | 0.0339 |
| SMN L_SupFS OCC L_AOcS | YFAS_SymptomCount | −0.3489 | 0.0112 | 0.0448 | 0.0411 |
| DMN L_PosDCgG DMN L_InfTS | YFAS_TimeSpent | 0.3862 | 0.0047 | 0.0375 | 0.0339 |
| DMN L_PrCun OCC L_LinG | YFAS_TimeSpent | −0.3664 | 0.0075 | 0.0397 | 0.0362 |
| DMN R_MTG DMN L_AngG | YFAS_TimeSpent | 0.3599 | 0.0088 | 0.0397 | 0.0377 |
| DMN R_SupTS DMN R_MTG | YFAS_TimeSpent | −0.3322 | 0.0161 | 0.0488 | 0.0443 |

Brain vs Metabolite

| Variable 1 | Variable 1 | r | p | p-adjusted | FDR |
|---|---|---|---|---|---|
| DMN R_MTG CEN R_IntPS_TrPS | 1-methylnicotinamide | 0.5053 | 0.0001 | 0.0102 | 0.0251 |
| SMN L_InfPrCS SMN L_PosCS | 2′-deoxyguanosine | −0.3447 | 0.0123 | 0.0475 | 0.0425 |
| CAN L_OrG DMN L_SupTS | arachidoylcarnitine (C20) | 0.3807 | 0.0054 | 0.0375 | 0.0354 |
| CEN R_MFG SMN R_PosCG | arachidoylcarnitine (C20) | −0.3717 | 0.0067 | 0.0386 | 0.0362 |
| DMN L_PrCun OCC L_CcS | arachidoylcarnitine (C20) | −0.3393 | 0.0139 | 0.0488 | 0.0443 |
| CEN R_SupPL DMN R_SuMarG | biocytin | 0.4084 | 0.0026 | 0.0334 | 0.0316 |
| DMN L_PosDCgG DMN L_InfTS | biocytin | −0.3826 | 0.0051 | 0.0375 | 0.0353 |
| SMN L_InfPrCS SMN L_PosCS | biocytin | −0.3637 | 0.0080 | 0.0397 | 0.0376 |
| DMN R_SuMarG DMN R_MTG | cytosine | −0.4060 | 0.0028 | 0.0334 | 0.0316 |
| DMN R_SupTS SMN L_SupFG | cytosine | −0.4027 | 0.0031 | 0.0334 | 0.0316 |
| DMN R_MTG DMN L_TPl | cytosine | −0.3769 | 0.0059 | 0.0375 | 0.0359 |
| SMN R_SupFG CAN R_OrG | cytosine | −0.3729 | 0.0065 | 0.0383 | 0.0362 |
| DMN R_AngG DMN R_MTG | docosapentaenoate (n6 DPA; 22:5n6) | 0.3703 | 0.0069 | 0.0389 | 0.0362 |
| DMN R_MTG OCC L_SupOcS_TrOcS | glycosyl ceramide (d18:2/24:1, d18:1/24:2) | 0.3499 | 0.0110 | 0.0446 | 0.0410 |
| OCC R_SupOcG CEN L_POcS | glycosyl-N-(2-hydroxynervonoyl)-sphingosine (d18:1/24:1(2OH)) | −0.3361 | 0.0149 | 0.0488 | 0.0443 |
| SAL R_MPosCgG_S SMN R_SbCG_S | glycosyl-N-stearoyl-sphingosine (d18:1/18:0) | 0.3352 | 0.0151 | 0.0488 | 0.0443 |
| DMN L_PosDCgG DMN L_InfTS | glycylisoleucine | −0.3842 | 0.0049 | 0.0375 | 0.0348 |
| DMN R_AngG OCC L_SupOcG | glycylisoleucine | 0.3380 | 0.0143 | 0.0488 | 0.0443 |
| DMN L_PosDCgG DMN L_InfTS | glycylvaline | −0.3338 | 0.0156 | 0.0488 | 0.0443 |
| CEN R_SupPL DMN R_SuMarG | histidine | 0.3607 | 0.0086 | 0.0397 | 0.0377 |
| DMN R_SuMarG DMN R_MTG | homocitrulline | −0.3873 | 0.0046 | 0.0375 | 0.0339 |
| DMN R_MTG DMN R_SuMarG | homocitrulline | −0.3589 | 0.0090 | 0.0397 | 0.0377 |
| CEN R_MFG SMN R_PosCG | hyocholate | −0.3813 | 0.0053 | 0.0375 | 0.0354 |
| OCC R_SupOcG OCC R_Cun | hypoxanthine | 0.3759 | 0.0060 | 0.0375 | 0.0359 |
| CEN R_SupPL DMN R_SuMarG | leucine | 0.3786 | 0.0056 | 0.0375 | 0.0359 |
| CEN L_SupPL OCC L_Cun | lignoceroylcarnitine (C24) | 0.4082 | 0.0027 | 0.0334 | 0.0316 |
| CEN R_SupPL DMN R_SuMarG | lignoceroylcarnitine (C24) | 0.3321 | 0.0162 | 0.0488 | 0.0443 |
| SMN R_SupFG CAN R_OrG | maltose | −0.3334 | 0.0157 | 0.0488 | 0.0443 |
| CEN R_MFG SMN R_PRCG | N-acetyl-beta-glucosaminylamine | −0.3570 | 0.0094 | 0.0397 | 0.0377 |
| CEN R_SupPL DMN R_SuMarG | phenylalanine | 0.3654 | 0.0077 | 0.0397 | 0.0365 |
| DMN R_SupTS SMN L_SupFG | phenylalanine | −0.3367 | 0.0147 | 0.0488 | 0.0443 |
| CEN R_SupPL DMN R_SuMarG | proline | 0.4369 | 0.0012 | 0.0283 | 0.0260 |
| CEN R_SupPL DMN R_SuMarG | serine | 0.3749 | 0.0062 | 0.0375 | 0.0359 |
| CEN R_MFG SMN R_PosCG | stearoylcarnitine (C18) | −0.4473 | 0.0009 | 0.0269 | 0.0260 |
| OCC R_MOcG OCC R_SupOcS_TrOcS | succinate | 0.3886 | 0.0044 | 0.0375 | 0.0339 |
| CEN R_SupPL OCC R_SupOcG | succinate | 0.3434 | 0.0127 | 0.0482 | 0.0433 |
| DMN L_SupTS DMN L_TPl | succinate | 0.3405 | 0.0135 | 0.0488 | 0.0441 |
| CAN L_OrG DMN L_SupTS | taurolithocholate | 0.3565 | 0.0095 | 0.0397 | 0.0377 |
| CEN R_SupPL DMN R_SuMarG | threonine | 0.4021 | 0.0031 | 0.0334 | 0.0316 |
| DMN L_SupTS DMN L_TPl | tyramine | 0.4363 | 0.0012 | 0.0283 | 0.0260 |
| CEN R_SupPL OCC R_SupOcG | tyramine | 0.4205 | 0.0019 | 0.0332 | 0.0302 |
| DMN R_SupTS SMN L_SupFG | tyrosine | −0.3593 | 0.0089 | 0.0397 | 0.0377 |
| CEN R_SupPL DMN R_SuMarG | tyrosine | 0.3449 | 0.0123 | 0.0475 | 0.0425 |

| | | | | | |
|---|---|---|---|---|---|
| Variable 1 | Variable 2 | r | p | p-adjusted | FDR |

Brain vs Microbiome

| | | | | | |
|---|---|---|---|---|---|
| DMN L_PrCun OCC L_CcS | [Ruminococcus] gvus group | −0.3788 | 0.0056 | 0.0375 | 0.0359 |
| SAL R_MPosCgG_S SMN R_SbCG_S | [Ruminococcus] gvus group | −0.3527 | 0.0103 | 0.0426 | 0.0400 |
| OCC R_LinG DMN L_PrCun | [Ruminococcus] gvus group | −0.3408 | 0.0134 | 0.0488 | 0.0440 |
| DMN R_MTG DMN L_TPl | Acidaminococcus | −0.3576 | 0.0093 | 0.0397 | 0.0377 |
| DMN R_MTG DMN L_TPl | Fournierella | 0.4342 | 0.0013 | 0.0283 | 0.0260 |
| DMN R_MTG DMN L_AngG | Fournierella | −0.4017 | 0.0032 | 0.0334 | 0.0316 |
| DMN R_MTG DMN L_TPl | Fournierella | 0.3567 | 0.0094 | 0.0397 | 0.0377 |
| DMN R_MTG CEN R_IntPS_TrPS | Fournierella | −0.3318 | 0.0163 | 0.0488 | 0.0443 |
| SMN R_SupFG CAN R_OrG | Lachnospira | −0.3685 | 0.0072 | 0.0394 | 0.0362 |
| CEN R_MFG DMN R_MTG | Lachnospira | 0.3597 | 0.0088 | 0.0397 | 0.0377 |
| DMN R_PrCun OCC R_SupOcG | Prevotella | 0.4317 | 0.0014 | 0.0283 | 0.0270 |
| OCC R_SupOcG DMN L_MTG | Prevotella | 0.3410 | 0.0134 | 0.0488 | 0.0440 |

Metabolite vs Clinical

| | | | | | |
|---|---|---|---|---|---|
| tyramine | BCope_Acceptance | −0.4135 | 0.0023 | 0.0334 | 0.0316 |
| succinate | BCope_Acceptance | −0.3952 | 0.0037 | 0.0367 | 0.0324 |
| succinate | YFAS_GivenUp | 0.4117 | 0.0024 | 0.0334 | 0.0316 |
| succinate | YFAS_LossControl | 0.5653 | 0.0000 | 0.0015 | 0.0038 |
| succinate | YFAS_TimeSpent | 0.3911 | 0.0038 | 0.0367 | 0.0324 |

Metabolite vs Microbiome

| | | | | | |
|---|---|---|---|---|---|
| hyocholate | [Ruminococcus] gvus group | 0.4903 | 0.0002 | 0.0102 | 0.0251 |
| N-acetyl-1-methylhistidine | Lachnospira | 0.5745 | 0.0000 | 0.0014 | 0.0033 |
| taurolithocholate | Lachnospira | 0.4645 | 0.0004 | 0.0196 | 0.0260 |
| hyocholate | Lachnospira | 0.4030 | 0.0025 | 0.0334 | 0.0316 |
| stearoylcarnitine (C18) | Lachnospira | 0.3842 | 0.0041 | 0.0375 | 0.0335 |
| tyramine | Prevotella | 0.4484 | 0.0007 | 0.0233 | 0.0260 |
| ribulose/xylulose | Prevotella | 0.4140 | 0.0019 | 0.0332 | 0.0301 |

Moderate Physical Activity vs. Low Physical Activity

| | | | | | |
|---|---|---|---|---|---|
| Variable 1 | Variable 2 | r | p | p-adjusted | FDR |

Brain vs Clinical

| | | | | | |
|---|---|---|---|---|---|
| CEN L_MFG DMN L_PrCun | YFAS_ContinuedUse | 0.4843 | 0.0007 | 0.0202 | 0.0260 |
| DMN R_SupTS DMN R_AngG | YFAS_LossControl | 0.3605 | 0.0150 | 0.0370 | 0.0443 |
| CEN L_MFG DMN L_PrCun | YFAS_SymptomCount | 0.4261 | 0.0035 | 0.0276 | 0.0319 |
| DMN R_SupTS SMN L_SupFG | YFAS_TimeSpent | 0.4275 | 0.0034 | 0.0276 | 0.0319 |
| DMN R_SupTS DMN R_AngG | YFAS_TimeSpent | 0.3823 | 0.0096 | 0.0334 | 0.0377 |

Brain vs Metabolites

| | | | | | |
|---|---|---|---|---|---|
| OCC R_SupOcG CEN R_POcS | 1-methylnicotinamide | −0.3612 | 0.0148 | 0.0370 | 0.0443 |
| OCC R_MOcG OCC R_SupOcS_TrOcS | 1-methylnicotinamide | 0.3605 | 0.0150 | 0.0370 | 0.0443 |
| SMN R_SupFG CAN R_OrG | argininate | −0.3932 | 0.0075 | 0.0325 | 0.0362 |
| DMN R_SupTS ERN L_PaHipG | argininate | −0.3646 | 0.0138 | 0.0370 | 0.0443 |
| DMN R_SupTGLp DMN R_MTG | argininate | −0.3580 | 0.0158 | 0.0370 | 0.0443 |
| DMN R_MTG DMN L_SupTGLp | argininate | −0.3562 | 0.0163 | 0.0374 | 0.0443 |
| DMN R_MTG DMN L_SupTGLp | aspartate | −0.3716 | 0.0120 | 0.0369 | 0.0425 |
| OCC R_SupOcG DMN L_PrCun | biocytin | 0.3889 | 0.0083 | 0.0334 | 0.0377 |
| OCC R_Cun DMN L_PrCun | biocytin | 0.3636 | 0.0141 | 0.0370 | 0.0443 |
| DMN L_SupTS OCC L_SupOcS_TrOcS | glycosyl ceramide (d18:2/24:1, d18:1/24:2) | 0.3980 | 0.0068 | 0.0325 | 0.0362 |
| CEN L_MFG SMN L_SupFG | glycosyl ceramide (d18:2/24:1, d18:1/24:2) | 0.3863 | 0.0088 | 0.0334 | 0.0377 |
| DMN R_MTG DMN L_TPl | glycylisoleucine | −0.4878 | 0.0007 | 0.0202 | 0.0260 |
| OCC R_Cun DMN L_PrCun | glycylisoleucine | 0.4829 | 0.0008 | 0.0202 | 0.0260 |
| DMN R_SuMarG DMN R_MTG | glycylisoleucine | −0.4400 | 0.0025 | 0.0274 | 0.0316 |
| OCC R_SupOcG DMN L_PrCun | glycylisoleucine | 0.4220 | 0.0039 | 0.0285 | 0.0324 |
| DMN R_MTG DMN R_SuMarG | glycylisoleucine | −0.3878 | 0.0085 | 0.0334 | 0.0377 |
| DMN R_MTG DMN L_TPl | glycylisoleucine | −0.3633 | 0.0142 | 0.0370 | 0.0443 |
| OCC R_SupOcG DMN L_PrCun | glycylvaline | 0.4727 | 0.0010 | 0.0202 | 0.0260 |
| OCC R_Cun DMN L_PrCun | glycylvaline | 0.4697 | 0.0011 | 0.0202 | 0.0260 |
| DMN R_MTG DMN L_SupTGLp | glycylvaline | −0.4268 | 0.0035 | 0.0276 | 0.0319 |
| DMN R_SuMarG DMN R_MTG | glycylvaline | −0.4159 | 0.0045 | 0.0294 | 0.0339 |
| DMN R_MTG DMN L_TPl | glycylvaline | −0.3950 | 0.0073 | 0.0325 | 0.0362 |
| OCC R_SupOcG CEN R_POcS | histidine | 0.4171 | 0.0044 | 0.0294 | 0.0339 |
| DMN R_SupTS DMN R_MTG | histidine | −0.3938 | 0.0074 | 0.0325 | 0.0362 |
| DMN R_MTG CEN R_IntPS_TrPS | histidine | 0.3669 | 0.0132 | 0.0370 | 0.0440 |
| DMN R_MTG DMN L_SupTGLp | histidine | −0.3666 | 0.0133 | 0.0370 | 0.0440 |
| SMN R_SupFG CEN R_MFG | histidine | −0.3573 | 0.0160 | 0.0370 | 0.0443 |
| OCC R_MOcG OCC R_SupOcS_TrOcS | hyocholate | 0.4026 | 0.0061 | 0.0325 | 0.0359 |
| DMN R_SuMarG DMN R_MTG | hyocholate | −0.3669 | 0.0132 | 0.0370 | 0.0440 |
| OCC R_SupOcG DMN L_PrCun | leucine | 0.4539 | 0.0017 | 0.0232 | 0.0301 |
| DMN R_MTG DMN L_SupTGLp | leucine | −0.4294 | 0.0032 | 0.0276 | 0.0319 |
| OCC R_Cun DMN L_PrCun | leucine | 0.3941 | 0.0074 | 0.0325 | 0.0362 |
| DMN R_MTG DMN L_TPl | leucine | −0.3600 | 0.0151 | 0.0370 | 0.0443 |
| SMN R_SupFG CAN R_OrG | maltose | −0.3576 | 0.0159 | 0.0370 | 0.0443 |
| OCC R_SupOcG DMN L_PrCun | methionine sulfoxide | 0.4679 | 0.0012 | 0.0202 | 0.0260 |
| OCC R_Cun DMN L_PrCun | methionine sulfoxide | 0.3989 | 0.0066 | 0.0325 | 0.0362 |
| OCC R_Cun DMN L_PrCun | N-acetyl-beta-glucosaminylamine | 0.4008 | 0.0064 | 0.0325 | 0.0362 |
| DMN R_MTG DMN L_SupTGLp | phenylalanine | −0.4547 | 0.0017 | 0.0232 | 0.0301 |
| OCC R_SupOcG DMN L_PrCun | phenylalanine | 0.4317 | 0.0031 | 0.0276 | 0.0316 |
| DMN R_SuMarG DMN R_MTG | phenylalanine | −0.4282 | 0.0033 | 0.0276 | 0.0319 |
| DMN R_MTG DMN L_TPl | phenylalanine | −0.4126 | 0.0049 | 0.0297 | 0.0347 |
| OCC R_Cun DMN L_PrCun | phenylalanine | 0.3852 | 0.0090 | 0.0334 | 0.0377 |
| DMN R_SupTS DMN R_AngG | phenylalanine | −0.3754 | 0.0110 | 0.0363 | 0.0410 |
| DMN R_MTG DMN L_SupTGLp | proline | −0.3712 | 0.0121 | 0.0369 | 0.0425 |
| CEN R_SupPL OCC R_SupOcG | ribulose/xylulose | −0.3840 | 0.0092 | 0.0334 | 0.0377 |
| CEN L_MFG DMN L_PrCun | ribulose/xylulose | −0.3585 | 0.0156 | 0.0370 | 0.0443 |
| CEN L_MFG SMN L_SupFG | ribulose/xylulose | −0.3578 | 0.0158 | 0.0370 | 0.0443 |
| OCC R_SupOcG DMN L_PrCun | serine | 0.4843 | 0.0007 | 0.0202 | 0.0260 |
| DMN R_MTG DMN L_SupTGLp | serine | −0.4353 | 0.0028 | 0.0276 | 0.0316 |
| OCC R_Cun DMN L_PrCun | serine | 0.4142 | 0.0047 | 0.0294 | 0.0339 |
| DMN R_SuMarG DMN R_MTG | serine | −0.3663 | 0.0133 | 0.0370 | 0.0440 |
| DMN R_MTG DMN L_TPl | serine | −0.3631 | 0.0142 | 0.0370 | 0.0443 |
| CAN L_OrG DMN L_SupTS | succinate | 0.4763 | 0.0009 | 0.0202 | 0.0260 |
| SMN L_SupFS OCC L_AOcS | succinate | 0.3776 | 0.0106 | 0.0357 | 0.0404 |
| DMN R_MTG CEN R_IntPS_TrPS | taurolithocholate | 0.4428 | 0.0023 | 0.0268 | 0.0316 |
| OCC R_SupOcG DMN L_PrCun | threonine | 0.4527 | 0.0018 | 0.0232 | 0.0301 |
| DMN R_MTG DMN L_SupTGLp | threonine | −0.3840 | 0.0092 | 0.0334 | 0.0377 |
| OCC R_Cun DMN L_PrCun | threonine | 0.3707 | 0.0122 | 0.0369 | 0.0425 |
| CEN R_POcS OCC R_SupOcG | tyramine | −0.3959 | 0.0071 | 0.0325 | 0.0362 |
| OCC R_SupOcG DMN L_PrCun | tyrosine | 0.4472 | 0.0021 | 0.0253 | 0.0316 |
| OCC R_Cun DMN L_PrCun | tyrosine | 0.4055 | 0.0057 | 0.0325 | 0.0359 |
| DMN R_MTG DMN L_SupTGLp | tyrosine | −0.4046 | 0.0058 | 0.0325 | 0.0359 |
| DMN R_SuMarG DMN R_MTG | tyrosine | −0.3795 | 0.0101 | 0.0348 | 0.0395 |
| DMN R_MTG DMN L_TPl | tyrosine | −0.3705 | 0.0122 | 0.0369 | 0.0425 |

Brain vs Microbiome

| | | | | | |
|---|---|---|---|---|---|
| DMN L_PosDCgG DMN L_InfTS | Lachnospira | −0.5017 | 0.0004 | 0.0202 | 0.0260 |
| DMN R_MTG DMN L_TPl | Bacteroides | −0.4951 | 0.0005 | 0.0202 | 0.0260 |
| DMN R_SuMarG DMN R_MTG | Bacteroides | −0.4888 | 0.0007 | 0.0202 | 0.0260 |
| DMN R_MTG DMN R_SuMarG | Bacteroides | −0.4803 | 0.0008 | 0.0202 | 0.0260 |
| DMN L_SuMarG CEN L_SupPL | Prevotella | −0.4244 | 0.0037 | 0.0278 | 0.0324 |
| OCC R_SupOcG CEN R_POcS | Lachnospiraceae ND3007 group | 0.4199 | 0.0041 | 0.0290 | 0.0335 |
| DMN R_MTG DMN R_SuMarG | Prevotella | 0.4108 | 0.0051 | 0.0301 | 0.0353 |
| OCC R_SupOcG CEN R_POcS | Faecalibacterium | 0.3979 | 0.0068 | 0.0325 | 0.0362 |
| OCC R_SupOcG OCC R_Cun | Lachnospiraceae ND3007 group | 0.3937 | 0.0075 | 0.0325 | 0.0362 |
| SMN L_InfPrCS SMN L_PosCS | Lachnospira | 0.3900 | 0.0081 | 0.0334 | 0.0376 |
| DMN R_MTG DMN L_TPl | Bacteroides | −0.3825 | 0.0095 | 0.0334 | 0.0377 |
| CEN L_MFG SMN L_SupFG | Lachnospira | −0.3695 | 0.0125 | 0.0370 | 0.0428 |
| OCC R_SupOcG OCC R_Cun | Faecalibacterium | 0.3607 | 0.0149 | 0.0370 | 0.0443 |

Metabolite vs Microbiome

| | | | | | |
|---|---|---|---|---|---|
| 2′-deoxyguanosine | Bacteroides | 0.5072 | 0.0004 | 0.0202 | 0.0260 |
| methionine sulfoxide | Bacteroides | 0.4650 | 0.0013 | 0.0203 | 0.0260 |
| glycylisoleucine | Bacteroides | 0.4149 | 0.0046 | 0.0294 | 0.0339 |
| glycylvaline | Bacteroides | 0.3712 | 0.0121 | 0.0369 | 0.0425 |
| N-acetyl-1-methylhistidine | Fusicatenibacter | 0.3585 | 0.0156 | 0.0370 | 0.0443 |
| proline | Lachnospira | −0.4316 | 0.0031 | 0.0276 | 0.0316 |
| threonine | Lachnospira | −0.4034 | 0.0060 | 0.0325 | 0.0359 |
| leucine | Lachnospira | −0.3829 | 0.0094 | 0.0334 | 0.0377 |
| serine | Lachnospira | −0.3743 | 0.0113 | 0.0366 | 0.0412 |
| biocytin | Lachnospira | −0.3621 | 0.0145 | 0.0370 | 0.0443 |
| methionine sulfoxide | Lachnospira | −0.3584 | 0.0156 | 0.0370 | 0.0443 |
| proline | Lachnospiraceae ND3007 group | −0.3753 | 0.0111 | 0.0363 | 0.0410 |
| ribulose/xylulose | Prevotella | 0.3883 | 0.0084 | 0.0334 | 0.0377 |
| methionine sulfoxide | Prevotella | −0.3873 | 0.0086 | 0.0334 | 0.0377 |

High Physical Activity vs. Moderate Physical Activity

| | | | | | |
|---|---|---|---|---|---|
| Variable 1 | Variable 2 | r | p | p-adjusted | FDR |

Brain vs Clinical

| | | | | | |
|---|---|---|---|---|---|
| CEN L_SbPS ERN L_InfFS | Education | −0.4236 | 0.0006 | 0.0292 | 0.0260 |
| CAN L_OrG DMN L_SupTS | Education | −0.3997 | 0.0013 | 0.0292 | 0.0260 |
| DMN L_SupTS OCC L_SupOcS_TrOcS | Education | −0.3715 | 0.0030 | 0.0292 | 0.0316 |
| OCC R_SupOcG DMN L_SupTS | Education | −0.3711 | 0.0030 | 0.0292 | 0.0316 |
| DMN R_SupTS ERN L_PaHipG | Education | 0.3661 | 0.0034 | 0.0311 | 0.0319 |
| DMN R_SupTS DMN R_MTG | Education | −0.3360 | 0.0076 | 0.0359 | 0.0362 |
| CEN L_MFG DMN L_PrCun | Education | −0.3225 | 0.0106 | 0.0424 | 0.0404 |
| SMN R_SupFS CEN R_MFG | Education | −0.3203 | 0.0111 | 0.0424 | 0.0410 |

Brain vs Metabolite

| | | | | | |
|---|---|---|---|---|---|
| OCC R_SupOcG CEN L_POcS | 1-methylnicotinamide | −0.3309 | 0.0071 | 0.0359 | 0.0362 |
| CEN R_POcS OCC R_SupOcG | 1-methylnicotinamide | −0.3097 | 0.0120 | 0.0443 | 0.0425 |
| SMN L_InfPrCS SMN L_PosCS | 2′-deoxyguanosine | −0.3019 | 0.0145 | 0.0477 | 0.0443 |
| OCC R_MOcG OCC R_SupOcS_TrOcS | argininate | 0.3790 | 0.0018 | 0.0292 | 0.0301 |
| DMN R_SupTS DMN R_SupTS | argininate | 0.3330 | 0.0067 | 0.0359 | 0.0362 |
| CEN L_SbPS ERN L_InfFS | argininate | −0.2967 | 0.0164 | 0.0478 | 0.0443 |
| DMN R_MTG DMN L_SupTGLp | aspartate | −0.3551 | 0.0037 | 0.0311 | 0.0324 |
| CEN R_SupPL OCC R_SupOcG | biocytin | 0.3676 | 0.0026 | 0.0292 | 0.0316 |
| DMN R_AngG DMN R_MTG | docosapentaenoate (n6 DPA; 22:5n6) | 0.3380 | 0.0059 | 0.0359 | 0.0359 |
| DMN L_SupTS OCC L_SupOcS_TrOcS | glycosyl ceramide (d18:2/24:1, d18:1/24:2) | 0.3810 | 0.0017 | 0.0292 | 0.0301 |
| DMN R_MTG OCC L_SupOcS_TrOcS | glycosyl ceramide (d18:2/24:1, d18:1/24:2) | 0.3536 | 0.0039 | 0.0311 | 0.0324 |
| DMN L_SupTS OCC L_SupOcS_TrOcS | glycosyl-N-stearoyl-sphingosine (d18:1/18:0) | 0.3506 | 0.0042 | 0.0319 | 0.0335 |
| DMN R_MTG OCC L_SupOcS_TrOcS | glycosyl-N-stearoyl-sphingosine (d18:1/18:0) | 0.3429 | 0.0052 | 0.0359 | 0.0353 |
| OCC R_SupOcG DMN L_PrCun | glycylisoleucine | 0.3283 | 0.0076 | 0.0359 | 0.0362 |
| OCC R_Cun DMN L_PrCun | glycylisoleucine | 0.3220 | 0.0089 | 0.0394 | 0.0377 |
| DMN R_MTG DMN L_SupTGLp | glycylvaline | −0.3029 | 0.0142 | 0.0477 | 0.0443 |
| DMN R_SupTS SMN L_SupFG | histidine | −0.3072 | 0.0128 | 0.0450 | 0.0434 |
| CEN L_SbPS ERN L_InfFS | hyocholate | −0.3401 | 0.0056 | 0.0359 | 0.0359 |
| OCC R_SupOcG CEN L_POcS | hyocholate | −0.3133 | 0.0110 | 0.0424 | 0.0410 |
| OCC R_SupOcG OCC R_Cun | leucine | 0.3148 | 0.0106 | 0.0424 | 0.0404 |
| CEN R_SupPL OCC R_SupOcG | leucine | 0.2972 | 0.0162 | 0.0478 | 0.0443 |
| CEN R_MFG CEN R_IntPS_TrPS | lignoceroylcarnitine (C24) | 0.3418 | 0.0053 | 0.0359 | 0.0354 |
| OCC R_SupOcG CEN L_POcS | maltose | −0.4012 | 0.0009 | 0.0292 | 0.0260 |
| SMN R_SupFG CAN R_OrG | maltose | −0.3948 | 0.0011 | 0.0292 | 0.0260 |
| CEN L_SbPS ERN L_InfFS | maltose | −0.3718 | 0.0023 | 0.0292 | 0.0316 |
| CAN L_OrG DMN L_SupTS | maltose | −0.3006 | 0.0150 | 0.0477 | 0.0443 |
| OCC R_SupOcG CEN L_IntPS_TrPS | maltose | −0.2986 | 0.0157 | 0.0477 | 0.0443 |
| SMN R_SupFG CEN R_MFG | methionine sulfoxide | −0.3170 | 0.0101 | 0.0424 | 0.0395 |
| DMN R_MTG DMN L_SupTGLp | N-acetyl-1-methylhistidine | −0.3743 | 0.0021 | 0.0292 | 0.0316 |
| OCC R_SupOcG CEN L_POcS | N-acetyl-1-methylhistidine | −0.3254 | 0.0082 | 0.0374 | 0.0377 |
| DMN R_SupTS ERN L_PaHipG | N-acetyl-beta-glucosaminylamine | −0.3640 | 0.0029 | 0.0292 | 0.0316 |
| CEN R_MFG DMN R_CgSMarp | N-acetyl-beta-glucosaminylamine | 0.3639 | 0.0029 | 0.0292 | 0.0316 |
| SMN R_SupFG SMN R_PosCG | N-acetyl-beta-glucosaminylamine | 0.3322 | 0.0069 | 0.0359 | 0.0362 |
| DMN R_MTG CEN R_IntPS_TrPS | taurolithocholate | 0.4380 | 0.0003 | 0.0292 | 0.0260 |
| CEN R_SupPL OCC R_SupOcG | threonine | 0.3301 | 0.0072 | 0.0359 | 0.0362 |
| DMN R_MTG DMN L_SupTGLp | threonine | −0.3289 | 0.0075 | 0.0359 | 0.0362 |
| OCC R_SupOcG OCC R_Cun | threonine | 0.3089 | 0.0123 | 0.0443 | 0.0425 |
| DMN R_SupTS DMN R_AngG | threonine | −0.2992 | 0.0155 | 0.0477 | 0.0443 |
| DMN R_MTG OCC L_SupOcS_TrOcS | tyramine | −0.2996 | 0.0153 | 0.0477 | 0.0443 |

p-value significant<0.05.

### FDR=False Discovery Rate

### Brief Resilience Scale (BRS), Yale Food Addiction Scale (YFAS), Brief-COPE (Bcope)

Networks. SMN: sensorimotor, DMN: default mode, SAL: salience, CEN: central executive, CAN: central autonomic, ERN: emotion regulation, OCC: occipital.
Brain regions: L_SupPL=Left Superior parietal lobule (lateral part of P1), L_Cun=Left Cuneus (O6), R_AngG=Right Angular gyrus, R_SupTS=Right Superior temporal sulcus (parallel sulcus), R_MTG=Right Middle temporal gyrus (T2), L_SupTS=Left Superior temporal sulcus (parallel sulcus), L_AocS=Left Anterior occipital sulcus and preoccipital notch (temporo-occipital incisure), L_SbPS=Left Subparietal sulcus, L_InfFS=Left Inferior frontal sulcus, R_SupFS=Right Superior frontal sulcus, R_MFG=Right Middle frontal gyrus (F2), L_InfFS=Left Inferior frontal sulcus, L_AngG=Left Angular gyrus, L_PaHipG=Left Parahippocampal gyrus, parahippocampal part of the medial occipito-temporal gyrus, (T5), L_OrG=Left Orbital gyri, R_SuMarG=Right Supramarginal gyrus, R_SupPL=Right Superior parietal lobule (lateral part of P1), L_TP1=Left Planum temporal or temporal plane of the superior temporal gyrus, L_PosDCgG=Left Posterior-dorsal part of the cingulate gyrus (dPCC), L_InfTS=Left Inferior temporal sulcus, R_MFG=Right Middle frontal gyrus (F2), L_InfPrCS=Left Inferior part of the precentral sulcus, L_PosCS=Left Postcentral sulcus, L_LinG=Left Lingual gyrus, lingual part of the medial occipito-temporal gyrus, (O5), L_SupOcS_TrOcS=Left Superior occipital sulcus and transverse occipital sulcus, L_IntPS_TrPS=Left Intraparietal sulcus (interparietal sulcus) and transverse parietal sulci, R_AngG=Right Angular gyrus, R_SupOcG=Right Superior occipital gyrus (O1), L_PrCun=Left Precuneus (medial part of P1), L_MTG=Left Middle temporal gyrus (T2), R_SupTGLp=Right Lateral aspect of the superior temporal gyrus, L_SbOrS=Left Suborbital sulcus (sulcus rostrales, supraorbital sulcus), L_AcgG_S=Left Anterior part of the cingulate gyrus and sulcus (ACC), L_PrCun=Left Precuneus (medial part of P1), L_CcS=Left Calcarine sulcus, R_SupOcS_TrOcS=Right Superior occipital sulcus and transverse occipital sulcus, R_MocG=Right Middle occipital gyrus (O2, lateral occipital gyrus), R_OrG=Right Orbital gyri, R_SupFG=Right Superior frontal gyrus (F1), R_PrCun=Right Precuneus (medial part of P1), R_SupOcG=Right Superior occipital gyrus (O1), R_Cun=Right Cuneus (O6), R_MposCgG_S=Right Middle-posterior part of the cingulate gyrus and sulcus (pMCC), R_SbCG_S=Right Subcentral gyrus (central operculum) and sulci, L_MFG=Left Middle frontal gyrus (F2), L_SupTGLp=Left Lateral aspect of the superior temporal gyrus, L_SupFG=Left Superior frontal gyrus (F1), R_MTG=Right Middle temporal gyrus (T2), R_PRCG=Right Precentral gyrus, R_CgSMarp=Right Marginal branch (or part) of the cingulate sulcus, R_PosCG=Right Postcentral gyrus, L_SupTS=Left Superior temporal sulcus (parallel sulcus)

### Discussion

In this example, it is demonstrated that there are significant alterations associated with PA seen in the functional connectivity of the brain, beta diversity and relative abundance of the gut microbiome, and metabolites produced. These BGM system alterations are associated with improved psychosocial measures in an overweight and obese population. Given that individuals with high BMI face additional weight-related stressors compared to normal-weight individuals, these findings explore the possible utility of PA in preventing and treating mental illnesses in the high BMI population and how PA possibly promotes health beyond solely metabolic regulation.

An association was identified herein between higher PA and greater resilience, which is a protective factor against the development of psychiatric disorders such as depression and post-traumatic stress disorder (PTSD). Within YFAS food addiction measures, moderate PA was associated with the highest food addiction scores while high PA participants had the lowest scores, and these findings were associated with altered connectivity within brain regions of the DMN. Specifically, the moderate PA participants when compared to those with high PA had increased connectivity between the angular gyrus and middle temporal gyrus regions, which has been demonstrated to be increased in activation when participants passively viewed visual food cues versus while they actively inhibited the urge to eat. Previous studies on the chronic effects of exercise on appetite parameters have been largely conflicting, with some studies reporting an increase in subjective appetite in the fasted state after aerobic exercise, whereas others have reported a reduction or no change. These findings suggest that the subjective appetite responses to PA may be intensity-dependent, with moderate PA stimulating appetite and high intensity PA reducing appetite.

Individuals with lower PA were seen to have increased relative abundance of Blautia and Bacteroides, which have both been shown to be increased in patients with major depressive disorder (MDD) and bipolar disorder. Prevotella is decreased in patients with MDD, and it was seen herein that overall higher PA was associated with a positive fold change with Prevotella in both the high versus low and moderate versus low comparisons. Higher levels of Bacteroides and lower levels of Prevotella correlated with severity of anxiety. Therefore, the significantly increased Prevotella to Bacteroides ratio that we observed in both the moderate and high PA groups in comparison to the low PA group may suggest that increased physical activity is associated with microbiome signatures protective against depression and anxiety. In addition, it was found that low PA was associated with Ruminococcus gnavus, which can degrade mucins and lead to gut permeability. It has been shown that depression is associated with a low-grade intestinal inflammation, which may allow invasive bacteria to translocation into the systemic circulation. This can then trigger an increase in plasma immunoglobulins targeting these bacteria and could explain why clinical depression is accompanied by increases in IgA and/or IgM. Overall, these findings suggest that with more PA, individuals with higher BMI can encourage a microbiome signature that is protective against developing certain psychiatric illnesses such as depression and anxiety.

In addition to Prevotella's association with psychiatric disorders. Higher Prevotella was associated with more PA when comparing high versus low PA individuals, further demonstrating the crucial role of PA in metabolic and weight regulation through alterations in the microbiome. Fournierella was observed to have the greatest increase in relative abundance in the high PA versus the low PA group. In a population of participants with abdominal obesity, Fournierella has been found to be positively associated with a green-Mediterranean diet as well as reduced intrahepatic fat overtime. The increased abundance of these genera associated with lean-phenotypes and reduced intrahepatic fat further illustrate a widely accepted finding that more PA promotes a metabolically healthy microbiome that may prevent further weight gain in individuals who already have higher BMI.

It was also found that certain metabolites disclosed herein associated with PA in the context of cognitive health. In this study, a negative trend between the metabolite glycosyl-N-(2-hydroxynervonoyl)-sphingosine (d18:1/24:1 (20H)) and increased PA was observed. In a Puerto Rican study evaluating metabolites associated with cognitive function in a non-diabetic population, glycosyl-N-(2-hydroxynervonoyl)-sphingosine (d18:1/24:1 (20H)) was found to be related to poor cognition, with participants that scored higher on cognitive function having lower levels of glycosyl-N-(2-hydroxynervonoyl)-sphingosine (d18:1/24:1 (20H)). Additionally, herein, it was found that histidine levels were higher in the high and moderate PA groups when compared to the low PA group. Studies have shown that histidine intake improves cognitive function, potentially via the metabolism of histidine to histamine, and the histamine receptors (H1 and H3) in the brain are involved in functions related to anxiety, stress, appetite, and sleep. This suggests that increased PA potentially could contribute to promoting better cognitive function and mental health via influence on the metabolites.

PA-associated differences were also seen in brain functional connectivity, most notably when comparing the high versus moderate PA groups. The superior frontal gyrus (SupFG) and middle frontal gyrus (MFG), which are both frontal lobe regions implicated in general inhibitory control but also appetite control, were increased in connectivity in the high PA group compared to moderate PA, which is consistent with the clinical findings of lowest food cravings with high PA and highest food cravings with moderate PA. A possible explanation is that the MFG has also been proposed to act as a circuit-breaker between the ventral and dorsal attention networks, and thus allows for top-down reorientation of attention from endogenous stimuli such as hunger cues to exogenous stimuli in the environment. These findings demonstrate that in comparison to obese individuals, previously obese individuals who successfully maintained weight loss as well as lean individuals have greater activation in the SupFG in response to food cues and during tasks involving response inhibition. SupFG has also been negatively correlated to self-reported impulsivity, with ADHD individuals showing hypoactivity in SupFG and MFG. The finding of increased functional connectivity in the brain between these two regions was also correlated to the increased histidine we observed with PA, which supports the hypothesis that the effects of PA on appetite may be through connections within the gut-brain axis.

In addition, more emotional regulation network regions were increased in connectivity in moderate PA participants, and were linked to central autonomic versus central executive as seen in the high PA group, suggesting more cognitive control over emotional food cravings with more PA. There were also overall more CEN regions increased in connectivity with high PA linked to the somatosensory and default mode networks, in comparison to the increased connectivity between CEN and occipital regions seen in moderate PA individuals. This suggests more cognitive modulation and evaluation of sensory stimuli with high PA, that may contribute to more restraint and less impulsivity in uncontrolled eating.

Novel targets within the BGM system are identified herein for the prevention and treatment of various psychiatric conditions, which individuals with high BMI are already at higher risk for.

### Example 3: Supplemental Methods for Examples 1 and 2

### Questionnaire Methods

Various questionnaires were utilized to assess participant's physical activity levels and psychological well-being. Participants completed the validated International Physical Activity Questionnaire (IPAQ) long form, which comprises of 27 items that collected data in different domains (job-related, transport-related, domestic and leisure-time physical activity) and intensities (moderate, vigorous, walking) and includes sitting time. The Guidelines for Data Processing and Analysis of the IPAQ categorical scoring were used to determine participants' current level of physical activity and participants were grouped into low, moderate, or high physical activity level categories.

Additionally, psychological resiliency was assessed using the Brief Resilience Scale (BRS). To assess stress, anxiety, and mood, the Perceived Stress Scale (PSS), Hospital Anxiety and Depression Scale (HADS), State-Trait Anxiety Inventory (STAI), and the Positive and Negative Affect Schedule (PANAS) were used. The PSS is a 10-item scale used to measure stressful demands in a given situation, indicating that demands exceed ability to cope. The questions are based on participants reporting the frequency of their feelings within the past month to each question, which are scored on a scale of 0 (never) to 4 (very often). The HADS is a 14-item scale used to measure symptoms of anxiety and depression. The questions are scored on a scale of 0-3, corresponding to how much the individual identifies with the question for the past week. The STAI questionnaire is a 40-item form that assesses state anxiety (how the respondent feels right now at this moment) and trait anxiety (how the respondent “generally” feels). The PANAS survey is a 20-item scale comprising of 10 items measuring positive affect (e.g., excited, inspired) and 10 items measuring negative affect (e.g., upset, afraid). Each item is rated on a five-point Likert Scale, ranging from 1 (very slightly or not at all) to 5 (extremely), to measure the extent to which the affect has been experienced in a specified time frame. In order to assess effective and ineffective coping strategies, participants also completed the Brief-COPE questionnaire, which is a 28 item self-report questionnaire that comprises of 14 two-item subscales including: (1) self-distraction, (2) active coping, (3) denial, (4) substance use, (5) use of emotional support, (6) use of instrumental support, (7) behavioral disengagement, (8) venting, (9) positive reframing, (10) planning, (11) humor, (12) acceptance, (13) religion, and (14) self-blame.

Food addiction was assessed using the Yale Food Addiction Scale (YFAS) questionnaire, a 25-item scale developed to assess food addiction by assessing signs of substance-dependence symptoms in eating behavior. This scale is based upon the substance dependence criteria as found in the DSM-4 (e.g., tolerance [marked increase in amount; marked decrease in effect], withdrawal [agitation, anxiety, physical symptoms], and loss of control [eating to the point of feeling physical ill]). The YFAS questionnaire is a 25-question survey that measures several aspects of food addiction behavior: food dependence, withdrawal, tolerance, continued use despite problems, time spent eating, loss of control, inability to cut down, and clinically significant impairment. Food addiction was defined as having a YFAS symptom count ≥3 with clinically significant impairment or distress. Clinically significant impairment or distress was defined as having a at least one positive response to the following two questions in the YFAS questionnaire: “My behavior with respect to food and eating causes significant distress” and “I experience significant problems in my ability to function effectively (daily routine, job/school, social activities, family activities, health difficulties) because of food and eating,” similar to previously published works. The YFAS has displayed a good internal reliability (Kuder-Richardson α=0.86).

All patients underwent the UCLA Diet Checklist, which is a questionnaire developed by our institution, intended to represent the diet that best reflects what participants consume on a regular basis. The specific diets incorporated into this checklist were: Standard American (characterized by high consumption of processed, frozen, and packaged foods, pasta and breads, and red meat; vegetables and fruits are not consumed in large quantities), Modified American (high consumption of whole grains including some processed, frozen, and packaged foods; red meat is consumed in limited quantities; vegetables and fruit are consumed in moderate to large quantities), Mediterranean (high consumption of fruits, vegetables, beans, nuts, and seeds; olive oil is the key monounsaturated fat source; dairy products, fish, and poultry are consumed in low to moderate amounts and little red meat is eaten), and all other diets that do not fit into the above categories (vegan, vegetarian, and gluten-free). For data analysis, we combined standard American and modified American diet as one category. Mediterranean, and all other diets were combined as “other” for analysis.

Microbiome: Stool Collection, Processing, 16s rRNA Sequencing

Within 1 week of the participant's brain MRI scan, stool samples were collected and stored at −80° C. before 16S rRNA sequencing. DNA extraction was performed with the fecal samples using PowerSoil DNA Isolation Kit (MO BIO Laboratories, Carlsbad, CA) with bead beating following the manufacturers protocol. Using 515F and 806R primers, the V4 hypervariable region of the 16S rRNA gene were amplified. The PCR products were purified using the ZR-96 DNA Clean & Concentrator-5 Kit (Zymo Research, Irvine, CA) and subsequently sequenced with the Illumina HiSeq 2500 platform. Processing of base pair reads using QIIME v1.9.1 with default parameters and taxonomic sequences were assigned using closed reference operational taxonomic unit (OTU) picking in QIIME against the Greengenes database pre-clustered at 97% identity. If the OTUs were present in less than 10% of samples, they were removed. The median depth was 104,124 reads per sample with a standard deviation of 73,192 and a minimum read of 32,304.

DNA from stool was extracted using the DNA Fecal Microbe Miniprep Kit (Zymo Research). The V4 region of 16S ribosomal RNA was amplified and underwent paired end sequencing on an Illumina HiSeq 2500 (San Diego, CA, USA) as previously described. Sequences were processed through the DADA2 pipeline to generate exact amplicon sequence variants (ASVs) and taxonomy was assigned based upon the SILVA 138 database. Microbial alpha diversity was assessed on data rarefied to equal sequencing depth applying metrics including the Chaol index of richness and the Shannon index of evenness. Microbial composition (i.e. beta diversity) was compared across groups using robust Aitchison (a phylo-genetic distance metric) in QIIME2 and visualized with principal coordinates analysis. The significance of beta diversity, adjusting for covariates, was assessed using multivariate PERMANOVA with significance determined by 100,000 permutations.³⁰ Predicted metagenomics was performed using PICRUSt2 in QIIME2 using the default settings to predict abundances of bacterial gene families annotated as KEGG orthologs (KO) based on nearest reference genomes to 16S sequences.

Differential abundance of microbes was analyzed using MaAslin2, which utilizes a generalized linear mixed model with total sum scaling normalization for microbiome data. Predicted metagenome differences between groups was visualized through principal component analysis (PCA) and significance tested using PERMANOVA. Individual predicted genes were tested between groups using DESEq2 in R and corrected for multiple hypothesis testing using false discovery rate (FDR) correction (q<0.05 for significance). The raw sequences can be accessed NIH NCBI BioProject (BioProject ID: PRJNA946906).

### Metabolites

Using the same fecal samples as the 16S sequencing, samples were aliquoted under liquid nitrogen and then shipped to Metabolon. They were processed and analyzed as a single batch on Metabolon's global metabolomics and bioinformatics platform. Using established protocols, data was curated by mass spectroscopy as previously reported.

### Brain: MRI Acquisition

Whole brain structural and resting state functional connectivity data was acquired using a 3.0 T Siemens Prisma MRI scanner (Siemens, Erlangen, Germany).

Structural high resolution T1-weighted images were acquired: echo time/repetition time (TE/TR)=3.26 ms/2200 ms, field of view[25]=220×220 mm slice thickness=1 mm, 176 slices, 256×256 voxel matrices, and voxel size=0.86×0.86×1 mm.

Resting-state scans were acquired with eyes closed and an echo planar sequence with the following parameters: TE/TR=28 ms/2000 ms, flip angle=77 degrees, scan duration=10 m6 s, FOV=220 mm, slices=40 and slice thickness=4.0 mm, and slices were obtained with whole-brain coverage.

Preprocessing and quality control was done using Statistical Parametric Mapping-12 (SPM12) software and involved bias field correction, co-registration, motion correction, spatial normalization, tissue segmentation, and Fourier transformation for frequency distribution. Data was then spatially normalized to the Montreal Neurological Institute (MNI) template using the structural scans, and then smoothed using a 4 mm isotropic Gaussian kernel.

### Brain: Functional Network Construction

Functional brain networks were constructed as previously described. To summarize, measures of region-to-region functional connectivity (Fisher transformed Pearson's correlations) were computed using the CONN toolbox and the aCompCor method in Matlab. Confounding factors such as white matter, cerebrospinal fluid, the six motion realignment parameters, and the root mean squared values of the detrended realignment estimates were regressed out for each voxel using ordinary least squares regression on the normalized, smoothed resting-state images. Participants with RMS values over 0.25 were not included. Images were then filtered using a band-pass filter (0.008/s<f<0.08/s) to reduce the low and high-frequency noises. Although the influence of head motion cannot be completely removed, CompCor has been shown to be particularly effective for dealing with residual motion relative to other methods. Regions of interest were segmented with the Harvard-Oxford Subcortical atlases, the Schaefer 400 cortical atlas, and the Ascending Arousal Network brainstem atlas. These atlases parceled into a total of 430 brain regions. The ROI-ROI functional connectivity between the brain regions was indexed by a matrix of Fisher Z transformed correlation coefficients reflecting the association between average temporal BOLD time series signals across all voxels in each brain region. The magnitude of the Z value represents the weights in the functional network. Permuted statistical values from ROI-to-ROI analyses were further corrected using the false discovery rate (FDR) to measure significance with p(FDR)<0.05.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments are described herein. Such equivalents are intended to be encompassed by the following claims.

What is claimed is:

1. A method of improving mood and/or ameliorating a depressive symptom in a subject, the method comprising administering a composition comprising bacteria of at least one genus selected from Megasphaera, Methanobrevibacter, Christensenellaceae, Prevotella, Fournierella, Acidaminococcus and Akkermansia (e.g., wherein at least one genus is selected from Megasphaera, Methanobrevibacter, Christensenellaceae, Prevotella, and Akkermansia) to the subject.

2. The method of claim 1, wherein the Methanobrevibacter bacteria is Methanobrevibacter smithii.

3. The method of claim 1 or 2, wherein administration raises the levels of at least one genus of bacteria selected from Megasphaera, Methanobrevibacter, Christensenellaceae, Prevotella, Fournierella, Acidaminococcus and Akkermansia (e.g., wherein the at least one genus is selected from Megasphaera, Methanobrevibacter, Christensenellaceae, Prevotella, and Akkermansia) in the gastrointestinal (GI) tract of the subject.

4. The method of any one of the preceding claims, wherein the method further comprises administering an antibiotic to the subject prior to administration of Megasphaera, Methanobrevibacter, Christensenellaceae, Prevotella, Fournierella, Acidaminococcus or Akkermansia bacteria.

5. The method of any one of the preceding claims, wherein the method comprises administering bacteria of at least two genera selected from Megasphaera, Methanobrevibacter, Christensenellaceae, Prevotella, Fournierella, Acidaminococcus and Akkermansia (e.g., wherein at least two genera are selected from Megasphaera, Methanobrevibacter, Christensenellaceae, Prevotella, and Akkermansia) to the subject.

6. The method of any one of the preceding claims, wherein the method comprises administering bacteria of at least three genera selected from Megasphaera, Methanobrevibacter, Christensenellaceae, Prevotella, Fournierella, Acidaminococcus and Akkermansia (e.g., wherein at least three genera are selected from Megasphaera, Methanobrevibacter, Christensenellaceae, Prevotella, and Akkermansia) to the subject.

7. The method of any one of the preceding claims, wherein the method comprises administering bacteria of at least four genera selected from Megasphaera, Methanobrevibacter, Christensenellaceae, Prevotella, Fournierella, Acidaminococcus and Akkermansia (e.g., wherein at least four genera are selected from Megasphaera, Methanobrevibacter, Christensenellaceae, Prevotella, and Akkermansia) to the subject.

8. The method of any one of the preceding claims, wherein the method comprises administering Megasphaera, Methanobrevibacter, Christensenellaceae, Prevotella, Fournierella, Acidaminococcus and Akkermansia (e.g., administering Megasphaera, Methanobrevibacter, Christensenellaceae, Prevotella, and Akkermansia) bacteria to the subject.

9. A method of improving mood and/or ameliorating a depressive symptom in a subject, the method comprising administering a composition comprising myoinositol to the subject.

10. The method of claim 9, wherein the method further comprises administering an antibiotic to the subject prior to administration of myoinositol to the subject.

11. A method of improving mood and/or ameliorating a depressive symptom in a subject, the method comprising administering an agent to the subject that inhibits the activity of or lowers the levels of cytidine in the GI tract of the subject.

12. The method of claim 11, wherein the method further comprises administering an antibiotic to the subject prior to administration of the agent to the subject.

13. The method of claim 11 or claim 12, wherein the agent is a small molecule or a peptide.

14. A method of improving mood and/or ameliorating a depressive symptom in a subject, the method comprising administering a composition comprising at least one of the metabolites listed in Table 12 or Table 13 to the subject.

15. The method of claim 9, wherein the method further comprises administering an antibiotic to the subject prior to administration of at least one of the metabolites listed in Table 12 or Table 13 to the subject.

16. A method of improving mood and/or ameliorating a depressive symptom in a subject, the method comprising administering an agent to the subject that inhibits the activity of or lowers the levels of Ruminococcus gnavus in the GI tract of the subject.

17. The method of claim 16, wherein the agent is an antibiotic that target Ruminococcus gnavus.

18. The method of claim 16 or claim 17, wherein the agent is a small molecule or a peptide.

19. The method of any one of the preceding claims, wherein the depressive symptom is selected from anxiety, apathy, general discontent, guilt, hopelessness, loss of interest or pleasure in activities, mood swings, sadness, agitation, excessive crying, irritability, restlessness, social isolation, early awakening, excess sleepiness, insomnia, restless sleep, excessive hunger, fatigue, loss of appetite, lack of concentration, slowness in activity, suicidal ideation, weight gain and weight loss.

20. The method of any one of claims 1 to 19, wherein the subject is afflicted with or at risk for a psychiatric or neurologic disorder.

21. The method of any one of claims 1 to 20, wherein the subject is afflicted with or is at risk for bipolar depression.

22. The method of any one of claims 1 to 21, wherein the subject is afflicted with or is at risk for post-partum depression (PPD).

23. The method of any one of claims 1 to 22, wherein the subject is afflicted with or is at risk for post-traumatic stress disorder (PTSD).

24. The method of any one of claims 1 to 23, wherein the subject is afflicted with or is at risk for major depressive disorder (MDD).

25. The method of any one of claims 1 to 24, wherein the subject is afflicted with or is at risk for food addiction.

26. The method of any one of the preceding claims, wherein the composition is formulated for oral delivery.

27. The method of any one of the preceding claims, wherein the method comprises administering an additional therapy for a psychiatric or neurologic disorder.

28. A method for determining whether a subject is at risk for a psychiatric or neurologic disorder, the method comprising:
obtaining a sample from the GI tract of the subject, optionally isolating microbial DNA from the sample,
identifying the amount of at least one genus of bacteria selected from Megasphaera, Methanobrevibacter, Christensenellaceae, Prevotella, Fournierella, Acidaminococcus and Akkermansia (e.g., wherein the at least one genus is selected from Megasphaera, Methanobrevibacter, Christensenellaceae, Prevotella, and Akkermansia) in the microbiome of the sample,
and if the microbiome comprises a lower level of Megasphaera, Methanobrevibacter, Christensenellaceae, Prevotella, Fournierella, Acidaminococcus or Akkermansia bacteria compared to a control level, the subject is considered at risk for the psychiatric or neurologic disorder.

29. The method of claim 28, wherein the control level is a level measured in a sample from the GI tract of the subject taken earlier in time.

30. The method of claim 28, wherein the control level is a mean or median level of Megasphaera, Methanobrevibacter, Christensenellaceae, Prevotella, Fournierella, Acidaminococcus or Akkermansia bacteria in subjects not afflicted with or at risk for the psychiatric or neurologic disorder.

31. The method of any one of claims 28 to 30, wherein the psychiatric or neurologic disorder is depression, PPD, PTSD, MDD, food addiction, or bipolar disorder.

32. A method of treating or preventing a psychiatric or neurologic disorder in a subject, the method comprising administering a composition comprising bacteria of at least one genus selected from Megasphaera, Methanobrevibacter, Christensenellaceae, Prevotella, Fournierella, Acidaminococcus or Akkermansia (e.g., wherein the at least one genus is selected from Megasphaera, Methanobrevibacter, Christensenellaceae, Prevotella, and Akkermansia) to the subject.

33. A method of treating or preventing a psychiatric or neurologic disorder in a subject, the method comprising administering an agent to the subject that inhibits the activity of or lowers the levels of cytidine in the GI tract of the subject.

34. A method of treating or preventing a psychiatric or neurologic disorder in a subject, the method comprising administering a composition comprising myoinositol to the subject.

35. A method of treating or preventing a psychiatric or neurologic disorder in a subject, the method comprising administering a composition comprising at least one of the metabolites listed in Table 12 or Table 13 to the subject.

36. A method of treating or preventing a psychiatric or neurologic disorder in a subject, the method comprising administering an agent to the subject that inhibits the activity of or lowers the levels of Ruminococcus gnavus in the GI tract of the subject.

37. The method of any one of claims 32 to 36, wherein the psychiatric or neurologic disorder is depression, PPD, PTSD, MDD, food addiction, or bipolar disorder.

38. A method for treating or preventing a psychiatric or neurologic disorder in a subject, comprising administering a composition comprising a metabolite produced by a strain of Megasphaera, Methanobrevibacter, Christensenellaceae, Prevotella, Fournierella, Acidaminococcus or Akkermansia (e.g., wherein the at least one genus is selected from Megasphaera, Methanobrevibacter, Christensenellaceae, Prevotella, and Akkermansia) bacteria to the subject, e.g., a metabolite selected from bacterial culture supernatant, cell lysate, proteins, nucleic acids, lipids, and other bacterial molecules.

39. The method of claim 38, wherein the psychiatric or neurologic disorder is depression, PPD, PTSD, MDD, food addiction, or bipolar disorder.

40. A method of enhancing the beneficial effects of physical activity in a subject, the method comprising administering a composition comprising bacteria of at least one genus selected from Megasphaera, Methanobrevibacter, Christensenellaceae, Prevotella, Fournierella, Acidaminococcus or Akkermansia (e.g., wherein the at least one genus is selected from Megasphaera, Methanobrevibacter, Christensenellaceae, Prevotella, and Akkermansia) to the subject.

41. A method of enhancing the beneficial effects of physical activity in a subject, the method comprising administering an agent to the subject that inhibits the activity of or lowers the levels of cytidine in the GI tract of the subject.

42. A method of enhancing the beneficial effects of physical activity in a subject, the method comprising administering a composition comprising myoinositol to the subject.

43. A method of enhancing the beneficial effects of physical activity in a subject, the method comprising administering a composition comprising at least one of the metabolites listed in Table 12 or Table 13 to the subject.

44. A method of enhancing the beneficial effects of physical activity in a subject, the method comprising administering an agent to the subject that inhibits the activity of or lowers the levels of Ruminococcus gnavus in the GI tract of the subject.

45. The method of any one of claims 40 to 44 to 35, wherein the beneficial effects of physical activity include increased BRS resilience score, decreased Yale Food Addiction Scale (YFAS), improved mood, increased acceptance, or increased functional brain connectivity.

46. A method for treating or preventing a psychiatric or neurologic disorder in a subject, comprising administering a composition comprising a metabolite produced by a strain of Megasphaera, Methanobrevibacter, Christensenellaceae, Prevotella, Fournierella, Acidaminococcus or Akkermansia bacteria (e.g., a metabolite produced by a strain of Megasphaera, Methanobrevibacter, Christensenellaceae, Prevotella, or Akkermansia bacteria) to the subject, e.g., a metabolite selected from bacterial culture supernatant, cell lysate, proteins, nucleic acids, lipids, and other bacterial molecules.

47. The method of claim 46, wherein the beneficial effects of physical activity include increased BRS resilience score, decreased Yale Food Addiction Scale (YFAS), improved mood, increased acceptance, or increased functional brain connectivity.

48. The method of any one of the preceding claims, wherein the subject is a human.
